(19) United States
(12) Patent Application Publication
(10) Pub. No.: US 2026/0347438 A1
(43) Pub. Date: Nov. 5, 2026

(54) ANTIBODIES AGAINST SARBECOVIRUSES, THEIR VARIANTS AND USES THEREOF

(71) Applicant: The Trustees of Columbia University in the City of New York, New York, NY (US)

(72) Inventors: David D. HO, Bedford, NY (US); Lihong LIU, Fort Lee, NJ (US); Qian WANG, Fort Lee, NJ (US); Yicheng GUO, Fort Lee, NJ (US); Lawrence SHAPIRO, New York, NY (US); Ryan Gavin CASNER, Mechanicsburg, PA (US); Jian YU, New Providence, NJ (US)

(21) Appl. No.: 19/736,227

(22) Filed: Jul. 9, 2026

Related U.S. Application Data
(63) Continuation of application No. PCT/US25/11277, filed on Jan. 10, 2025.
(60) Provisional application No. 63/619,716, filed on Jan. 10, 2024.
(60) Provisional application No. 63/622,448, filed on Jan. 18, 2024.

(51) Int. Cl. C07K 16/104; A61K 45/06; A61P 31/14

(52) U.S. Cl. CPC C07K 16/104; A61K 45/06; A61P 31/14; C07K 2317/522; C07K 2317/53; C07K 2317/565; C07K 2317/567; C07K 2317/76

(57) ABSTRACT

The subject matter disclosed herein relates to engineered human monoclonal antibodies against Sarbecoviruses.

This application is a continuation of International Patent Application No. PCT/US2025/11277, filed on Jan. 10, 2025, which claims the benefit of and priority to U.S. Provisional Patent Application No. 63/619,716, filed on Jan. 10, 2024, and U.S. Provisional Patent Application No. 63/622,448, filed on Jan. 18, 2024, the contents of each of which are hereby incorporated by reference in their entireties.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### SEQUENCE LISTING

The instant application contains a Sequence Listing which has been submitted electronically in ASCII format and is hereby incorporated by reference in its entirety. Said ASCII copy, created on Jan. 8, 2025, is named 0019240.01338WO1_SL.txt and is 7345 bytes in size.

### BACKGROUND

Sarbecoviruses include SARS-CoV and SARS-CoV-2, as well as viruses similar to SARS-CoV and SARS-CoV-2, and clade 2 Sarbecoviruses.

### SUMMARY OF THE INVENTION

In certain embodiments, the subject matter described herein provides a monoclonal antibody or an antigen-binding fragment thereof comprising (i) a heavy chain variable domain (VH) and (ii) a light chain variable domain (VL), wherein the monoclonal antibody or antigen binding fragment thereof binds a portion of a receptor-binding domain (RBD) on a spike protein of a sarbecovirus.

In some embodiments, the monoclonal antibody or antigen-binding fragment thereof comprises a VH domain comprising a heavy chain CDR1 with at least 85%, 90%, 95%, or 99% identity to SEQ ID NO: 3, a heavy chain CDR2 with at least 85%, 90%, 95%, or 99% identity to SEQ ID NO: 4, and a heavy chain CDR3 with at least 85%, 90%, 95%, or 99% identity to SEQ ID NO: 5. In some embodiments, the monoclonal antibody or an antigen-binding fragment thereof comprises a VH domain comprising a heavy chain CDR1 identical to SEQ ID NO: 3, a heavy chain CDR2 identical to SEQ ID NO: 4, and a heavy chain CDR3 identical to SEQ ID NO: 5.

In some embodiments, the monoclonal antibody or antigen-binding fragment thereof comprises a VL domain comprising a light chain CDR1 with at least 85%, 90%, 95%, or 99% identity to SEQ ID NO: 6, a light chain CDR2 with at least 85%, 90%, 95%, or 99% identity to SEQ ID NO: 7, and a light chain CDR3 with least 85%, 90%, 95%, or 99% identity to SEQ ID NO: 8. In some embodiments, the monoclonal antibody or antigen-binding fragment comprises a VL domain comprising a light chain CDR1 identical to SEQ ID NO: 6, a light chain CDR2 identical to SEQ ID NO: 7, and a light chain CDR3 identical to SEQ ID NO: 8.

In some embodiments, the monoclonal antibody or antigen-binding fragment comprises framework regions of a VH domain which comprise at least 85%, 90%, 95%, or 99% identity to the framework regions of SEQ ID NO: 1. In some embodiments, the monoclonal antibody or antigen-binding fragment comprises framework regions of a VL domain which comprise at least 85%, 90%, 95%, or 99% identity to the framework regions of SEQ ID NO: 2. In some embodiments, the monoclonal antibody or antigen-binding fragment comprises the framework regions of a VH domain comprising framework regions that are derived from IGHV3-53 of FIG. 3A. In some embodiments, the monoclonal antibody or antigen-binding fragment thereof comprises the framework regions of a VL domain which comprise framework regions that are derived from IGKV3-11 of FIG. 3B. In some embodiments, the monoclonal antibody or antigen-binding fragment thereof of claims 1-5, wherein the framework regions of the VH domain comprise framework regions that are at least 85%, 90%, 95%, or 99% identical to the framework regions of IGHV3-53 of FIG. 3A. In some embodiments, the monoclonal antibody or antigen-binding fragment thereof comprises the framework regions of a VL domain which comprises framework regions that are at least 85%, 90%, 95%, or 99% identical to the framework regions of FIG. 3B.

In some embodiments, the monoclonal antibody or antigen-binding fragment comprises a VH domain which comprises at least 85%, 90%, 95%, or 99% identity to SEQ ID NO: 1. In some embodiments, the monoclonal antibody or antigen-binding fragment comprises a VH domain which comprises SEQ ID NO: 1. In some embodiments, the monoclonal antibody or antigen-binding fragment consists of a VH domain which comprises SEQ ID NO: 1.

In some embodiments, the monoclonal antibody or antigen-binding fragment thereof comprises a VL domain which comprises at least 85%, 90%, 95%, or 99% identity to SEQ ID NO: 2. In some embodiments, the monoclonal antibody or antigen-binding fragment thereof comprises a VL domain which comprises SEQ ID NO: 2. In some embodiments, the monoclonal antibody or antigen-binding fragment thereof comprises a VL domain which consists of SEQ ID NO: 2.

In some embodiments, the Sarbecovirus which the monoclonal antibody or antigen-binding fragment thereof binds to is a SARS-CoV or SARS-CoV-2. In some embodiments, the Sarbecovirus which the monoclonal antibody or antigen-binding fragment thereof binds to is a SARS-CoV-2 variant. In some embodiments, the SARS-CoV-2 variant which the monoclonal antibody or antigen-binding fragment thereof binds to is D614G, B.1.1.7, B.1.351, P.1, B.1.617.2, BA.1, BA.2, BA.5, BA.2.75, CH.1.1, DV.7.1, XBC.1.6, BQ.1.1, XBB.1.5, XBB.1.16.6, XBB.2.3, EG.5.1, FL.1.5.1, JF.1, HV.1, HK.3, BA.2.87.1, BA.2.86, JN.1, JN.4, LB.1, KP.2, KP.3, KP.2.3, KP.3.1.1, XEC, GX-pangolin, RaTG13, BANAL52, BANAL236, or GD-pangolin.

In some embodiments, the monoclonal antibody or antigen-binding fragment thereof comprises a VH domain comprising SEQ ID NO: 1 and a VL domain comprising SEQ ID NO:2. In some embodiments, the monoclonal antibody or antigen-binding fragment thereof comprising a VH domain comprises at least 85%, 90%, 95%, or 99% identity to SEQ ID NO: 1 and a VL domain comprising at least 85%, 90%, 95%, or 99% identity to SEQ ID NO:2.

In some embodiments, the monoclonal antibody or antigen-binding fragment thereof comprises a heavy chain CDR1, CDR2, and CDR3 of the monoclonal antibody of antigen-binding fragment thereof which has an increased RMSF score as compared to heavy chain CDR1, CDR2, and CDR3 of a monoclonal antibody or fragment thereof comprising SEQ ID NO:1 and SEQ ID NO: 2. In some embodiments, the average RMSF score is above 1.0. In some embodiments, the average RMSF score is above 1.2.

In some embodiments, the monoclonal antibody or antigen-binding fragment thereof comprises a VH domain which comprises at least 85%, 90%, 95%, or 99% identity to SEQ ID NO: 1 and comprises an amino acid mutation at position 71. In some embodiments, the VH domain comprises SEQ ID NO:1 comprising an amino acid mutation at position 71. In some embodiments, the VH domain consists of SEQ ID NO: 1 with an amino acid mutation at position 71. In some embodiments, the monoclonal antibody or antigen-binding fragment thereof comprises a VL domain which comprises at least 85%, 90%, 95%, or 99% identity to SEQ ID NO: 2. In some embodiments, the VL domain comprises SEQ ID NO: 2. In some embodiments, the VL domain consists of SEQ ID NO: 2.

In some embodiments, the amino acid mutation is R71A, R71C, R71D, R71E, R71F, R71G, R71H, R71I, R71K, R71L, R71M, R71N, R71P, R71Q, R71S, R71T, R71V, R71W, or R71Y. In some embodiments, the amino acid mutation is R71A. In some embodiments, the amino acid mutation is R71L. In some embodiments, the amino acid mutation is R71V.

In some embodiments, the monoclonal antibody or antigen-binding fragment thereof is a single chain antibody, Fab, Fab′, F(ab′)2, Fv or scFv. In some embodiments, the monoclonal antibody is an IgG type. In some embodiments, the monoclonal antibody is an IgA type.

In some embodiments, the monoclonal antibody or antigen-binding fragment thereof comprises a first arm comprising a VH domain and VL domain, wherein the VH and VL domain form a binding site that binds a portion of the RBD on the spike protein of a sarbecovirus; and a second arm comprising a second VH domain, identical to the VH domain of the first arm, and a second VL domain, identical to the VL domain of the first arm, wherein the second VH and second VL form a binding site that binds a portion of the RBD on the spike protein of a sarbecovirus. In some embodiments, the first arm and the second arm each further comprise a CH1 domain, a hinge domain, and a CL domain. In some embodiments, the VH domain of the first arm is encoded by a first polypeptide chain, the VL domain of the first arm is encoded by a second polypeptide chain, the second VH domain of the second arm is encoded by a third polypeptide chain, the second VL domain of the second arm is encoded by a fourth polypeptide chain. In some embodiments, the first polypeptide chain and the third polypeptide chain each further encode a hinge domain, a CH1 domain, the Fc domain, and wherein the second polypeptide chain and the fourth polypeptide chain each further encode a CL domain. In some embodiments, the first polypeptide chain and the third polypeptide chain comprise the same sequence, and the second polypeptide chain and the fourth polypeptide chain comprise the same sequence. In some embodiments, the first polypeptide chain and the second polypeptide chain are linked by one or more covalent disulfide bonds and the third polypeptide chain and the fourth polypeptide chain are linked by one or more covalent disulfide bonds, and the first polypeptide chain and the third polypeptide chain are linked by one or more covalent disulfide bonds. In some embodiments, the monoclonal antibody or antigen-binding fragment thereof comprises an engineered constant domain.

In certain aspects, the present application provides a pharmaceutical composition comprising any of the monoclonal antibody or antigen-binding fragment thereof described above; and a pharmaceutically acceptable carrier.

In certain aspects, the present application provides a method of treating or preventing a Sarbecovirus infection in a subject in need thereof, the method comprising administering to the subject a therapeutically effective amount of the pharmaceutical composition described above. In some embodiments, the Sarbecovirus is a SARS-CoV or SARS-CoV-2. In some embodiments, the Sarbecovirus is a SARS-CoV-2 variant. In some embodiments, the SARS-CoV-2 variant is D614G, B.1.1.7, B.1.351, P.1, B.1.617.2, BA.1, BA.2, BA.5, BA.2.75, CH.1.1, DV.7.1, XBC.1.6 variant, BQ.1.1, XBB.1.5, XBB.1.16.6, X13B.2.3, EG.5.1, FL.1.5.1, JF.1, HV.1, HK.3, BA.2.87.1, BA.2.86, JN.1, JN.4, LB.1, KP.2, KP.3, KP.2.3, KP.3.1.1, XEC, GX-pangolin, RaTG13, BANAL52, BANAL236, or GD-pangolin.

In some embodiments, the pharmaceutical composition is administered in combination with an anti-viral medicament. In some embodiments, the anti-viral medicament comprises a second anti-sarbecovirus antibody. In some embodiments, the second anti-sarbecovirus antibody comprises CYFN1006-146.

In certain aspects, the preset application provides a nucleic acid comprising a nucleotide sequence encoding any of the monoclonal antibody or antigen binding fragment thereof described above.

In certain aspects, the present application provides a vector comprising the nucleic acid molecule described above.

In certain aspects, the present application provides a cell comprising the vector described above.

In some embodiments, the cell expresses a monoclonal antibody or antigen-binding fragment thereof described above.

In certain embodiments, the present application provides a pharmaceutical composition comprising the nucleic acid described above. In some embodiments, the composition comprises one or more vectors comprising a nucleotide sequence encoding the monoclonal antibody or antigen binding fragment thereof described above.

In certain embodiments, the present application provides a means for binding a portion of the RBD on a spike protein of a sarbecovirus. In some embodiments, the means comprises any of the monoclonal antibody or antigen binding fragment thereof described above.

In certain aspects, the present application provides one or more vectors comprising a first vector comprising a nucleotide sequence encoding any of the VH chains described above a second vector comprising a nucleotide sequence encoding any of the VL chains described above. In some embodiments, the first and second vectors are the same vector. In some embodiments, the first and second vectors are two different vectors.

In certain aspects, the present application provides one or more host cells comprising a first vector comprising a polynucleotide sequence encoding any of the VH chains described above and a second vector comprising a polynucleotide sequence encoding any of the VH chains described above. In some embodiments, the first vector and the second vector are the same vector. In some embodiments, the first vector and the second vector are two different vectors.

### BRIEF DESCRIPTION OF FIGURES

The patent or application file contains at least one drawing originally in color. To conform to the requirements for PCT patent applications, many of the figures presented herein are black and white representations of images originally created in color.

FIGS. 1A-C show clinical information and cell sorting strategy. FIG. 1A shows clinical information of donor 19 whose blood sample was collected for neutralizing antibody evaluation and B cell sorting. FIG. 1B shows neutralization activity of serum from donor 19 against D614G, XBB.1.5, and SARS-CoV. Neutralization ID50 titer against each virus is denoted. FIG. 1C shows sorting strategy used to isolate XBB.1.5 spike and/or SARS-CoV spike specific B cells. B cells from Q2 and Q3 were collected and applied for the downstream 10× Genomic analysis. Numbers shown in the gates represent cell percentages.

FIGS. 2A-C show neutralization activity and epitope mapping of neutralizing monoclonal antibody (mAb) 19-77. FIG. 2A shows neutralization IC₅₀ values of 19-77 mAb against pseudotyped SARS-CoV-2 variants and SARS-CoV. FIG. 2B shows binding of 19-77 mAb to the indicated antigens tested by ELISA. Other antigens tested represent various portions of the spike protein including the full length Spike protein, the S1 subunit of the Spike protein, the S2 subunit of the Spike protein, the N terminal domain (NTD) of the S1 subunit, the receptor binding domain (RBD) of the S1 subunit, and the subdomain 1 of the RBD (RBD-SD1) of the S1 subunit. 19-77 binds the receptor binding domain (RBD) of the spike protein on the virus. FIG. 2C shows epitope mapping of 19-77 mAb by competition ELISA wherein substrates with known interactions with the spike protein compete with 19-77 mAb

FIGS. 3A-B show that genetic analyses show that 19-77 heavy chain is from VH3-53 family and light chain comes from VK3-11 family. FIG. 3A shows the 19-77 heavy chain variable region. FIG. 3B shows the 19-77 light chain variable region. Boxes indicate respective complementarity-determining regions (CDRs).

FIGS. 4A-B show global reconstruction of the mAb 19-77 with the D614G spike. Cryo-EM reconstruction of mAb 19-77 in complex with SARS-CoV-2 D614G spike at resolutions of 2.45 A°. FIG. 4A shows angle 1 of the reconstruction. FIG. 4B shows angle 2 of the reconstruction.

FIGS. 5A-C show spike protein escape mutations for 19-77 mAb. FIG. 5A shows that the emerging Omicron variants escape the neutralization by 19-77 mAb FIGS. 5B and 5C show that the structural modeling suggests that the escape mutations will cause steric hindrance between the spike protein and 19-77 mAb conferring resistance of the spike protein to the 19-77 mAbs.

FIG. 6 shows in silico optimization of 19-77-R71X in complex with HK.3 RBD. RBD, with R71X named as Ho71X mutation.

FIGS. 7A-B show neutralization activity of 19-77 mutants (Ho71X). FIG. 7A shows IC₅₀ (ug/mL) neutralization. FIG. 7B shows fold change in IC₅₀ relative to 19-77 mAb.

FIG. 8 shows neutralization of 19-77 mAb and 19-77 mutants in comparison with other SARS-CoV-2 mAbs.

FIG. 9 shows that R71/A/L/V mutations rescue the neutralizing activities of other VH3-53 and VH3-66 antibodies against SARS-CoV-2 variants.

FIGS. 10A-B show that R71 contacts with CDRH1 and CDRH2 by hydrogen bonds.

FIG. 11 shows comparison between Fab domains of 19-77 and its mutant 19-77_R71A in flexibility by root mean square fluctuation (RMSF). R71A increases the flexibility of CDRH1, CDRH2 and CDRH3 of 19-77.

FIGS. 12A-B shows comparative analysis of VH3-53 class antibodies with R71A mutation. FIG. 12A shows structural alignment of 10 VH3-53 class antibodies, highlighting the location of residue R71 in relation to CDRH1 and CDRH2. FIG. 12B shows changes in RMSF in the heavy chains of VH3-53 class antibodies. The Y-axis indicates the fold change in RMSF per residue for R71A compared to its corresponding wild-type antibody. The X-axis shows the residue positions within these antibodies. The graph features a line representing the average fold change in RMSF, with the grey shaded area indicating the standard deviation.

FIGS. 13A-E show neutralization activity and epitope mapping of 19-77. FIG. 13A shows neutralization IC₅₀ values of 19-77 against pseudotyped SARS-CoV-2 variants and SARS-CoV, with the IC₅₀ of each virus is denoted. Neutralization curves are shown as mean standard error of mean (SEM). FIG. 13B shows complementarity-determining region 3 (CDR3) lengths and somatic hypermutations (SHM) of 19-77 heavy chain (HC) and light chain (LC), compared with other 3-53 and 3-66 antibodies. 19-77 is highlighted in red. FIG. 13C shows cryo-EM reconstruction of 19-77 in complex with the SARS-CoV-2 D164G spike protein, at a resolution of 2.45 Å. FIG. 13D shows key interactions between 19-77 heavy chain and the D614G RBD. Hydrogen bonds are shown as the orange dashed lines. FIG. 13E shows key interactions between 19-77 light chain and the D614G RBD. Orange dashed lines indicate hydrogen bonds.

FIGS. 14 A-E shows sequence conservation and resistant mutations in the 19-77 epitope. FIG. 14A shows top view of the RBD inner face in complex with the 19-77 antibody. 19-77 heavy chain and light chain are shown in marine and light blue, respectively. The residues in the RBD are colored by the sequence entropy in circulating SARS-CoV-2 variants. The blue and cyan boundaries show the footprints of 19-77 and human ACE2 (hACE2), respectively. FIG. 14B shows structure modeling of how A475V on the RBD affects 19-77 neutralization. The clashes are shown as red plates. FIGS. 14 C-E show comparison of residues 455 and 456 on RBD and P100_{HC} in D614G (c), EG.5.1 (d) and HK.3 (e) structures. The van der Waals clashes are shown as green plates.

FIGS. 15A-E shows optimization of 19-77 in neutralization activity. FIG. 15A shows neutralization of 19-77 carrying individual mutations on both heavy and light chains, in comparison with the wildtype 19-77 antibody. FIG. 15B shows neutralization of 19-77 carrying various amino acid substitutions at R71 on the heavy chain, compared with the wildtype 19-77 antibody. FIG. 15C shows depiction of the interactions between R71 and the heavy chain CDRs H1 (cyan) and H2 (orange) in 19-77. Dashed lines indicate the presence of hydrogen bonds. FIG. 15D shows structure modeling of K71 (blue) and A71 (green) with HCDRs of 19-77. Dashed lines indicate the presence of hydrogen bonds between 19-77ΔK. FIG. 15E shows comparison of the RMSF between the Fab regions of 19-77 and its mutant 19-77ΔA. The Y-axis represents the fold change in RMSF per residue in 19-77ΔA relative to 19-77. The X-axis displays the positional numbering of residues within the antibody. RMSF, per-residue root-mean-square fluctuation.

FIG. 16 shows neutralization activity of 19-77 and 19-77ΔA/L/V against the indicated pseudoviruses. Neutralization IC₅₀ values of the indicated antibodies against a panel of pseudotyped viruses, including SARS-CoV-2 variants and SARS-CoV-2-like sarbecoviruses.

FIG. 17 shows R71A/L/V sensitize the neutralization activity of other VH3-53/66 class antibodies. Neutralization of the indicated antibodies from VH3-55/66 and other classes carrying R71A/L/V mutations, compared with the respective wildtype (WT) antibodies.

FIGS. 18A-E shows molecular basis of 19-77ΔV accommodating mutations in HK.3 and JD.1.1. FIG. 18A shows cryo-EM maps of 19-77 and 19-77ΔV Fabs bound to SARS-CoV-2 RBD variants. FIG. 18B shows distribution of Cα distance for the heavy chain residues of 19-77 in A compared with 19-77 in the D614G complex. The structures are aligned based on RBD. FIG. 18C shows comparison of CDRH1, CDRH2, and R71V in the alignments of 19-77 structures. The hydrogen bonds are shown as orange dashed lines. FIG. 18D shows interaction details of P100HC in 19-77 and 19-77ΔV with F455 and L456 in the HK.3 RBD. The orange and green dashed lines represent the distances between P100HC and F455 in 19-77 and 19-77ΔV, respectively. FIG. 18E shows interaction details of N32HC in 19-77 and 19-77ΔV with A475V in the D614G and JD.1.1 RBDs. The orange and green dashed lines represent the distances between V475 and N32 in 19-77 and 19-77ΔV, respectively.

FIGS. 19A-C shows in vitro selection of 19-77 resistant mutations. FIG. 19A shows properties of the authentic JN.1 escape variant selected under the pressure of 19-77ΔV. FIG. 19B shows properties of the replication-competent VSV-JN.1 escape variants selected under the pressure of 19-77ΔV. FIG. 19C shows structural modeling of how A475D, G476D, N487H, F456S, Y473S, and Y489H affect 19-77 neutralization. The clashes are shown as red plates, and the hydrogen bonds are shown as orange dashed lines.

FIGS. 20A-C shows clinical information and sorting strategy. FIG. 20A shows clinical information of donor 19, whose blood sample was collected for neutralizing antibody evaluation and B cell sorting. FIG. 20B shows neutralizing IC₅₀ values of the antibodies from donor 19 against D614G and EG.5.1. 19-77 is highlighted with a dotted line. FIG. 20C shows Germline genes, CDR3 amino acid sequences, and SHM percentages of 19-77 heavy and light chains.

FIGS. 21A-C show cryo-EM and X-ray data for mAbs 19-77 and 19-77ΔV in complex with SARS-CoV-2 spike trimers or RBDs. FIG. 21A shows global refinement Fourier Shell Correction curves showing the overall resolution of the indicated complexes. FIG. 21B shows Cryo-EM data collection and model refinement of the indicated complexes. FIG. 21C shows X-ray diffraction data collection and refinement statistics.

FIGS. 22A-D show neutralizing IC₅₀ values of 19-77 mutants. FIG. 22A shows free energy change (ΔΔG) for saturation mutagenesis of the 19-77 heavy chain. Blue represents beneficial mutations, while red indicates mutations with adverse effects. FIG. 22B shows neutralization IC₅₀ values of 19-77 carrying the indicated mutations in the heavy chain. FIG. 22C shows neutralization IC₅₀ values of 19-77 carrying the indicated mutations in the light chain. FIG. 22D shows neutralization IC₅₀ values of 19-77 carrying various amino acid substitutions at R71 in the heavy chain.

FIGS. 23A-C show biological properties of 19-77. FIG. 23A shows yields of 19-77 and 19-77ΔA/L/V from transiently transfected Expi293 cells at day 4 post-transfection. FIG. 23B shows size exclusion chromatography (SEC) profiles of 19-77 and 19-77ΔA/L/V antibodies. FIG. 23C shows pharmacokinetics of 19-77 and 19-77ΔA/L/V in mice over 10 days after intraperitoneal injection. Data are shown as mean±SEM (standard error of the mean).

FIG. 24 shows R71/A/L/V mutations rescue the neutralizing activities of VH3-53 and VH3-66 antibodies against SARS-CoV-2 escaping variants. The figure shows neutralization IC₅₀ values of the indicated antibodies and their R71A/L/V mutants.

FIGS. 25A-D show infectivity and neutralization activity of 19-77ΔV escape variants in the context of VSV pseudotyped viruses. FIG. 25A shows infectivity of the indicated pseudotyped escape variants selected by 19-77ΔV from authentic JN.1 in Vero-E6-TMPRSS2-T2A-ACE2 cells. Data are shown as mean±SEM (standard error of the mean). FIG. 25B shows infectivity of the indicated pseudotyped escape variants selected by 19-77ΔV from replication-competent VSV-JN.1 in Vero-E6-TMPRSS2-T2A-ACE2 cells. Data are shown as mean±SEM.

FIG. 25C shows neutralization IC₅₀ values of 19-77, 19-77ΔV, and hACE2 against the indicated pseudotyped escape variants. FIG. 25D shows neutralization IC₅₀ values of 19-77, 19-77ΔA/L/V, and hACE2 against the indicated pseudotyped escape variants.

### DETAILED DESCRIPTION

All patent applications, published patent applications, issued and granted patents, texts, and literature references cited in this specification are hereby incorporated herein by reference in their entirety to more fully describe the state of the art to which the present disclosed subject matter pertains.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting.

The singular forms “a”, “an” and “the” include plural reference unless the context clearly dictates otherwise.

Antibodies Against Sarbecoviruses and their Variants

Disclosed herein are novel monoclonal antibodies that, in some embodiments, are useful for preventing or treating disease (e.g., viral infection).

Unless otherwise indicated, the practice of the subject matter disclosed here can employ conventional techniques of cell biology, cell culture, molecular biology, transgenic biology, microbiology, recombinant DNA, and immunology, which are within the skill of the art. Such techniques are fully explained in the literature.

A protein is encoded by a nucleic acid (including, for example, genomic DNA, complementary DNA (cDNA), synthetic DNA, as well as any form of corresponding RNA). One skilled in the art can produce a protein in several ways, including, but not limited to isolating the protein via biochemical means or expressing a nucleotide sequence encoding the protein. The nucleic acids encoding a protein can be produced via recombinant DNA technology. Such recombinant nucleic acids can be prepared by conventional techniques, including chemical synthesis, genetic engineering, enzymatic techniques, or a combination thereof.

Protein variants can include amino acid sequence modifications. For example, amino acid sequence modifications can be substitutional, insertional or deletional variants. Insertions can include amino and/or carboxyl terminal fusions as well as intrasequence insertions of single or multiple amino acid residues. Insertions are generally smaller than those of amino or carboxyl terminal fusions, for example, on the order of one to four residues. Deletions are characterized by the removal of one or more amino acid residues from the protein sequence. Substitutions are characterized by the substitution of one or more amino acid residues in the protein sequence with another amino acid. These protein variants ordinarily are prepared by site-specific mutagenesis of nucleotides in the DNA encoding the protein, thereby producing DNA encoding the variant, and thereafter expressing the DNA in recombinant cell culture or using cell-free methods known in the art.

### Sarbecovirus

Sarbecoviruses are respiratory viruses that include SARS-CoV-2. In some embodiments, disclosed herein are monoclonal antibodies which bind sarbecoviruses. In some embodiments, the antibody can bind to a portion of the sarbecovirus. In some embodiments, the portion on the sarbecovirus which the antibody binds to is a subunit of the spike protein. In some embodiments, the subunit is the S1 subunit. In some embodiments, the antibody binds to a domain on the S1 subunit. In some embodiments, the S1 subunit domain is the N-terminal domain (NTD). In some embodiments, the S1 subunit domain is the receptor-binding domain (RBD). In some embodiments, the subunit is the S2 subunit. In some embodiments, the S2 subunit domain is the heptad repeat 1 (HR1) domain. In some embodiments, the S2 subunit domain is the heptad repeat 2 (HR2) domain. In some embodiments, the S2 subunit domain is the transmembrane domain (TM). In some embodiments, the S2 subunit domain is the cytoplasmic domain (CP).

In some embodiments, the sarbecovirus is SARS-CoV. In some embodiments the sarbecovirus is SARS-CoV-2. In some embodiments the sarbecovirus is SARS-CoV-2-D614G. In some embodiments the sarbecovirus is SARS-CoV-2 B.1.1.7 (alpha) variant. In some embodiments the sarbecovirus is SARS-CoV-2 B.1.351 (beta) variant. In some embodiments the sarbecovirus is SARS-CoV-2 P.1 (gamma) variant. In some embodiments the sarbecovirus is SARS-CoV-2 B.1.617.2 (delta) variant. In some embodiments the sarbecovirus is SARS-CoV-2 BA.1 variant. In some embodiments the sarbecovirus is SARS-CoV-2 BA.2 variant. In some embodiments the sarbecovirus is SARS-CoV-2 BA.5 variant. In some embodiments the sarbecovirus is SARS-CoV-2 BA.2.75 variant. In some embodiments the sarbecovirus is SARS-CoV-2 CH.1.1 variant. In some embodiments the sarbecovirus is SARS-CoV-2 DV.7.1 variant. In some embodiments the sarbecovirus is SARS-CoV-2 XBC.1.6 variant. In some embodiments the sarbecovirus is SARS-CoV-2 BQ.1.1. In some embodiments the sarbecovirus is SARS-CoV-2 XBB.1.5 variant. In some embodiments the sarbecovirus is SARS-CoV-2 XBB.1.16.6 variant. In some embodiments the sarbecovirus is SARS-CoV-2 XBB.2.3 variant. In some embodiments the sarbecovirus is SARS-CoV-2 EG.5.1 variant. In some embodiments the sarbecovirus is SARS-CoV-2 FL.1.5.1 variant. In some embodiments the sarbecovirus is SARS-CoV-2 JF.1 variant. In some embodiments the sarbecovirus is SARS-CoV-2 HV.1 variant. In some embodiments the sarbecovirus is SARS-CoV-2 HK.3 variant. In some embodiments the sarbecovirus is SARS-CoV-2 HK.3 varoamt. In some embodiments the sarbecovirus is SARS-CoV-2 JD.1.1 variant. In some embodiments the sarbecovirus is SARS-CoV-2 BA.2.87.1 variant. In some embodiments the sarbecovirus is SARS-CoV-2 BA.2.86 variant. In some embodiments the sarbecovirus is SARS-CoV-2 JN.1 variant. In some embodiments the sarbecovirus is SARS-CoV-2 JN.4 variant. In some embodiments the sarbecovirus is SARS-CoV-2 LB.1 variant. In some embodiments the sarbecovirus is SARS-CoV-2 KP.2 variant. In some embodiments the sarbecovirus is SARS-CoV-2 KP.3 variant. In some embodiments the sarbecovirus is SARS-CoV-2 KP.2.3 variant. In some embodiments the sarbecovirus is SARS-CoV-2 KP.3.1.1 variant. In some embodiments the sarbecovirus is SARS-CoV-2 XEC variant. In some embodiments the sarbecovirus is the SARS-CoV-2 GX-pangolin variant. In some embodiments the sarbecovirus is the SARS-CoV-2 RaTG13 variant. In some embodiments the sarbecovirus is SARS-CoV-2 BANAL52 variant. In some embodiments the sarbecovirus is SARS-CoV-2 BANAL236 variant. In some embodiments the sarbecovirus is SARS-CoV-2 GD-pangolin variant.

Since the emergence of COVID-19 in late 2019, its causative agent, SARS-CoV-2, has undergone considerable evolution driven by immune pressure, with each successive viral variant becoming more resistant to serum antibodies elicited by prior infection and/or vaccination. By late 2020, mutations in the viral spike protein resulted in a discernible antigenic drift that yielded the Alpha variant, which evaded antibodies targeting the antigenic supersite on the N-terminal domain (NTD) (see Example 1 References 1-3). Shortly thereafter, further antigenic drift led to the Beta and Gamma variants that evaded not only NTD-directed antibodies but also some antibodies directed to so-called class 1 and class 2 regions of the receptor-binding domain (RBD) (see Example 1 References 1, 4, 5). These variants were in turn rapidly replaced by the Delta variant that dominated much of 2021, and it evaded antibodies directed to select class 2 and class 3 epitopes on the RBD6. Importantly, this initial wave of SARS-CoV-2 variants knocked out three therapeutic monoclonal antibodies (mAbs) authorized to treat COVID-19 infection—etesevimab, bamlanivimab, and casirvimab (see Example 1 References 6-10).

The first major SARS-CoV-2 antigenic shift occurred with emergence of the Omicron (BA. 1) variant in late 2021 in southern Africa (see Example 1 References 11, 12), likely due to a saltatory event resulting in an accumulation of 34 spike mutations. This variant rapidly gain dominance in the population, as a consequence of its exceptional resistance to serum antibodies in the population (see Example 1 References 13-18). Specifically, mutations in Omicron impaired the neutralizing activity of antibodies targeting all regions (classes 1-4) of the RBD, as well as two additional clinically authorized mAbs—imdevimab and tixagevimab (see Example 1 References 9, 13, 19). Antigenic drift from BA.1 then ensued successively throughout 2022 and yielded subvariants BA.2, BA.5, and BQ.1.1, each being more antibody resistant than its predecessor. This evolutionary drift also knocked out the utility of three more clinically authorized mAbs—sotrovimab, ciligavimab, and bebtelovimab (see Example 1 References 19-23).

The second major antigenic shift was detected in late 2022 when a recombination event between BA.2 and BA.2.75 resulted in the XBB subvariant, which promptly evolved into XBB.1.5, EG.5.1, HK.3, and HV.1 via further antigenic drift (see Example 1 References 11, 24-26). Again, each progeny subvariant was more antibody resistant than its predecessor, largely by evading more antibodies directed to the class 1 region of RBD. Sequentially, these subvariants dominated much of 2023 until the third major antigenic shift led to the emergence of BA.2.86, which presumably was due to another saltatory event, again in southern Africa, leading to an accumulation of >30 mutations in the BA.2 spike (see Example 1 References 27). This new subvariant was again highly resistant to serum neutralizing antibodies, including those targeting the subdomain 1 (SD-1) region of spike (see Example 1 References 28-30). With one additional spike mutation, L455S, BA.2.86 became JN.1 that has since gained dominance worldwide from late 2023 until recently (see Example 1 References 31, 32).

A monoclonal antibody known as pemivibart (Permagard or VYD222) is authorized for clinical use as pre-exposure prophylaxis for immunocompromised individuals who do not respond robustly to COVID-19 vaccines (see Example 1 References 33). This antibody is engineered to have broad activity against many known SARS-CoV-2 strains, including JN.1. However, as the JN.1 sublineage continued its antigenic drift, new forms such as KP.2 and KP.3 began to replace their predecessor in recent months. Currently, the dominant SARS-CoV-2 is KP.3.1.1, and the emergent XEC is steadily gaining ground (see Example 1 References 34). Alarmingly, these subvariants are already ~18-28-fold more resistant to pemivibart in vitro than JN.1 (see Example 1 References 35). This looming threat to the only authorized pre-exposure prophylaxis for millions of immunocompromised individuals who live in fear of COVID-19 is a stark reminder of the necessity to continue to develop more medical interventions to keep up with SARS-CoV-2 evolution.

In some embodiments, described herein is the isolation and characterization of a human monoclonal antibody, 19-77. Structural analyses and mutational scanning led to the construction of engineered antibody variants, 19-77Δ, with a single amino-acid substitution at residue R71 in the framework region 3 of the heavy chain. These modifications resulted in greater flexibility of the complementarity-determining regions (CDRs) of the heavy chain while binding to the viral spike, thereby enabling three 19-77Δ variants to neutralize all SARS-CoV-2 strains tested with much improved potency and breadth, including against the prevalent JN.1 sublineages. The subject matter disclosed herein not only offers new antibodies for use inpre-exposure prophylaxis against COVID-19 for immunocompromised individuals, but also introduces a unique engineering approach to improve antibody activity.

Monoclonal Antibodies (iAbs)

An antibody can be produced by expressing DNA encoding the antibody in recombinant cell culture or using cell-free methods known in the art followed by purification. Non-limiting purification methods include size exclusion chromatography, ammonium sulfate fractionation, ion exchange chromatography, affinity chromatography, and preparative gel electrophoresis. An antibody can also be produced by isolating the antibody from a blood sample from a subject who is producing the antibody due to infection or inoculation.

In some embodiments, the antibody may be post-translationally modified. In some embodiments, polypeptides or proteins can be modified enzymatically or chemically, by addition or removal of one or more post-translational modifications. For example, a polypeptide or protein can be glycosylated or deglycosylated enzymatically. Polypeptides can be phosphorylated using a purified kinase, such as a MAP kinase (e.g, p38, ERK, or INK) or a tyrosine kinase (e.g., Src or erbB2). A polypeptide or protein can also be modified through synthetic chemistry. Alternatively, one can isolate the antibody from a cell or tissue that expresses the polypeptide with the desired post-translational modification. In another embodiment, a nucleic acid molecule encoding the polypeptide or protein of interest is introduced into a host cell. The host cell is capable of post-translationally modifying the encoded polypeptide(s) in the desired fashion. If the polypeptide or protein does not contain a motif for a desired post-translational modification, one can alter the post-translational modification by mutating the nucleic acid sequence of a nucleic acid molecule encoding the polypeptide(s) so that it contains a site for the desired post-translational modification. The nucleic acid molecule can also be introduced into a host cell that is capable of post-translationally modifying the encoded polypeptide(s). One can also delete sites that are post-translationally modified by mutating the nucleic acid sequence so that the encoded polypeptide(s) does not contain the post-translational modification motif, or by introducing the native nucleic acid molecule into a host cell that is not capable of post-translationally modifying the encoded polypeptide(s).

Useful post-synthetic and post-translational modifications include conjugation to chemical moieties. Amine-reactive and thiol-reactive fluorophore derivatives have been synthesized that react under nondenaturing conditions with N-terminal amino groups and epsilon amino groups of lysine residues, on the one hand, and with free thiol groups of cysteine residues, on the other. Chemical moieties that usefully can be conjugated to antibodies disclosed herein include radioactive labels, echosonographic contrast reagents, and MRI contrast agents. Kits are available commercially that permit conjugation of proteins to a variety of amine-reactive or thiol-reactive chemical moieties.

There are five classes of human antibodies—IgA, IgD, IgE, IgG, and IgM. Each class has various isotypes, e.g., IgG1, IgG2, IgG3, IgG4, IgAQ1, and IgA2. In some embodiments, the antibodies disclosed herein belong to the IgG class. IgG can be divided into four subclasses: IgG1, IgG2, IgG3, and IgG4. Each subclass has a unique profile with respect to antigen binding, immune complex formation, complement activation, triggering of effector cells, half-life, and placental transport. E.g., see Gestur Vidarsson, et al., IgG Subclasses and Allotypes: From Structure to Effector Functions, 5 Frontiers in Immunology 520 (2014), incorporated by reference herein in its entirety. In some embodiments, the antibodies disclosed herein belong to the IgG1 class. The term “immunoglobulin” (Ig) is used interchangeably with “antibody” herein.

The IgG immunoglobulin molecule consists of four polypeptide chains. There are two identical light (L) chains and two identical heavy (H) chains. Disulfide bonds connect the four chains in a “Y” configuration where the light chains bracket the heavy chains starting at the bottom end of the “Y” and continuing through the variable region to the dual ends of the “Y”. Each L chain is linked to an H chain by one covalent disulfide bond. The two H chains are linked to each other by one or more disulfide bonds depending on the H chain isotype. Each H and L chain also has regularly spaced intrachain disulfide bridges. Each heavy chain consists of an N-terminal variable domain (VH) and three constant domains (CH1, CH2, CH3). There is an additional “hinge region” between CH1 and CH2. Similarly, the light chains consist of an N-terminal variable domain (VL) and a constant domain (CL). The variable domains of the heavy chain and light chain may be referred to as “VH” and “VL”, respectively. These domains are the most variable parts of the antibody and contain the antigen binding sites. The VL is aligned with the VH and the CL is aligned with the first constant domain of the heavy chain (CH1). The pairing of a VH and VL together forms a single antigen-binding site. The part of the antibody formed by the lower hinge region and the CH2/CH3 domains of the heavy chain is called “Fc” (“fragment crystalline”). See e.g., Basic and Clinical Immunology, 8th Edition, Daniel P. Sties, Abba I. Terr and Tristram G. Parsolw (eds), Appleton & Lange, Norwalk, CT, 1994, page 71 and Chapter 6, incorporated by reference herein in its entirety.

The variability in an antibody sequence is focused in three segments called complementarity determining regions (CDRs) or hypervariable regions (HVRs). There are CDRs are in the light-chain variable domain and in the heavy-chain variable domain. The more highly conserved portions of the variable domains are called the framework regions (FR). The variable domains of the heavy and light chains each comprise four FR regions. The FR domains generally adopt a beta-sheet configuration, connected by three CDRs, which mainly form loops connecting the beta-sheet structure of the FR regions. In some instances, the three CDRs form parts of the beta-sheet. The CDRs in each chain are held together by the FR regions and, with the CDRs from the other chain, contribute to the formation of the antigen-binding site of antibodies. See Kabat et al, Sequences of Immunological Interest, Fifth Edition, National Institute of Health, Bethesda, MD (1991), incorporated by reference in its entirety herein. The constant domains are not involved directly in the binding of antibodies to an antigen. However, they exhibit various effector functions, such as the participation of the antibody in antibody-dependent cellular toxicity.

The antibodies disclosed herein (e.g., monoclonal antibodies) include, but are not limited to, full-length antibodies. In some embodiments, they also include other immunologically reactive/antigen-binding molecules. The antibodies of the various embodiments disclosed herein can include one or more of monoclonal antibodies, synthetic antibodies, engineered antibodies, oligoclonal or polyclonal antibodies, multiclonal antibodies, recombinantly produced antibodies, intrabodies, monospecific antibodies, monovalent antibodies, multispecific antibodies, multivalent antibodies, bispecific antibodies, bivalent antibodies, tetravalent antibodies, human antibodies, humanized antibodies, chimeric antibodies, CDR-grafted antibodies, primatized antibodies, Fab fragments, F(ab′) fragments, F(ab′)₂ fragments, Fv fragments, single-chain FvFcs (scFv-Fc), single-chain Fvs (scFv), Dabs, nanobodies, anti-idiotypic (anti-Id) antibodies, and any other immunologically-reactive/antigen-binding molecules. In some embodiments, the antibody disclosed herein is a monoclonal antibody.

In some embodiments, the monoclonal antibody comprises a first, second, third and fourth chain. In some embodiments, the first and third chains each comprise a VH domain. In some embodiments, the second and fourth chains each comprise a VL domain. In some embodiments, the first and third chains each further comprise a CH1 domain, a hinge domain, and a Fc domain. In some embodiments, the second and fourth chains each further comprise a CL domain. The pairing of the VH and VL of the first and second chains together forms a single antigen-binding site that in some embodiments binds to a spike protein on a sarbecovirus, such as, but not limited to, binding to a portion of the receptor binding domain (RBD) of the spike protein. The VL of the third and fourth chains together forms a single antigen-binding site specific for the same epitope(s) on the spike protein. In some embodiments, the first and second chains are linked by one or more covalent disulfide bonds. The third and fourth chains are linked by one or more covalent disulfide bonds. In some embodiments, the first and third chains are linked by one or more disulfide bonds.

Other immunologically reactive/antigen-binding molecules are also contemplated herein. A person of skill in the art can readily synthesize such molecules using the sequences and identified domains of the heavy and light chains of the antibodies disclosed herein. For example, in some embodiments, the monoclonal antibody comprises a first and second chain that associate together. In some embodiments, the first chain and second chain each comprises an scFv with specificity for an epitope on a spike protein on a sarbecovirus, such as, but not limited to, binding to a portion of the receptor binding domain (RBD) of the spike protein. An scFv comprises a variable heavy domain and variable light chain domain separated by a linker. In some embodiments, the linker is a glycine-serine linker.

In some embodiments, the monoclonal antibodies disclosed herein contain various modifications, substitutions, additions, or deletions to the variable or binding regions of the antibody. In some embodiments, the monoclonal antibodies disclosed herein contain substitutions or modifications of the constant region. In some embodiments, the antibodies disclosed herein contain one or more additional amino acid residue substitutions, mutations and/or modifications. These may result in a compound with preferred characteristics including, but not limited to: altered pharmacokinetics, increased serum half-life, increased binding affinity, reduced binding affinity, reduced immunogenicity, increased production, altered Fc ligand binding, enhanced or reduced ADCC or CDC activity, altered glycosylation and/or disulfide bonds and modified binding specificity.

### Sequences of the 19-77 Monoclonal Antibody

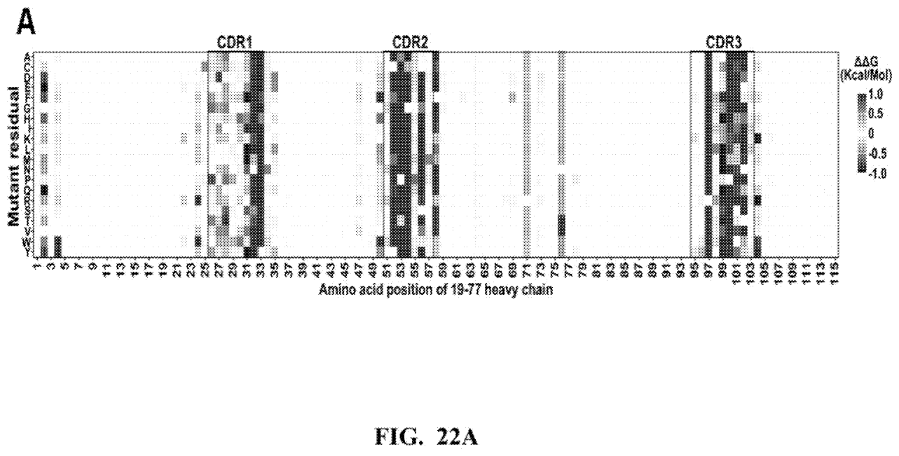

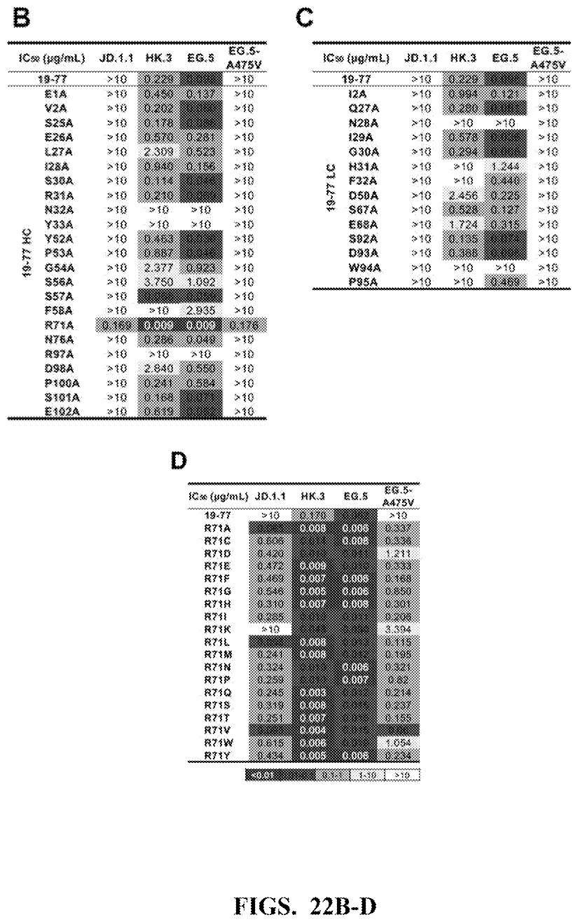

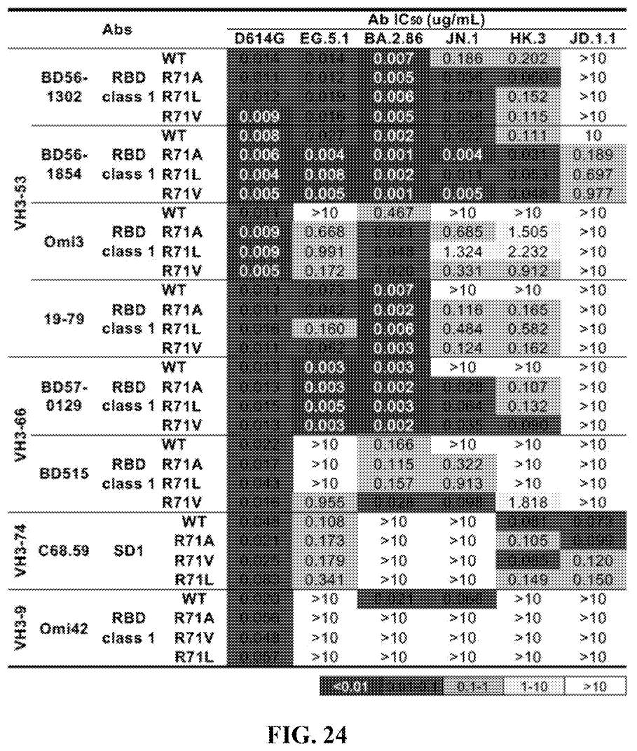

In certain aspects, the subject matter disclosed herein provides a monoclonal antibody, or an antigen-binding fragment thereof, comprising (i) a heavy chain (HC) variable domain (VH) and (ii) a light chain (LC) variable domain (VL). In some embodiments, the antibody disclosed herein binds a portion of the spike protein on a sarbecovirus. In some embodiments, the antibody disclosed herein binds a portion of the receptor-binding domain (RBD) of a spike protein of a sarbecovirus. In some embodiments, the antibody disclosed herein binds a portion of another domain on a spike protein of a sarbecovirus, e.g N-terminal domain (NTD), SD1, SD2, S1, S2, transmembrane domain (TMD), cytoplasmic domain. In some embodiments, the antibody comprises two polypeptide heavy chains each comprising a variable heavy chain (VH) domain, CH1 domain, a hinge domain, a Fc domain (CH2-CH3). In some embodiments, the antibody comprises two polypeptide light chains comprising a variable light chain (VL) domain and a CL domain. In some embodiments, the antibody disclosed herein is mAb 19-77 comprising SEQ ID NOs 1-8, as outlined in Table 1 below. In some embodiments, the antibody disclosed herein is a mutant variant of mAb 19-77. In some embodiments, the antibodies described herein are the 19-77 antibody variants described in FIGS. 22B-D. In some embodiments, the antibodies disclosed herein are the antibody variants described in FIG. 24

TABLE 1
Sequence of mAb 19-77.

| | |
|---|---|
| 19-77 heavy chain | EVQVVESGGGLIQPGGSLRLSCTAS |
| | ELIISRNYMSWVRQAPGKGLEWLS |
| | VIYPGGSSFYTDSLKGRFTISRDN |
| | SKNTLYLQMNRLGVEDTAIYYCVR |
| | DAPSESDWGQGTLVTVSS |
| | (SEQ ID NO: 1) |
| | Note: |
| | R71 residue is in bold. |
| 19-77 light chain | EIVLTQSPATLSLFPGERATLSCRA |
| | SQNIGHELTWYQQKPGQAPRLLIYD |
| | ASNRATGVPARFSGSGSETEFTLTI |
| | SSLGPEDFAVYYCQERSDWPRGTFG |
| | QGTKVEIK |
| | (SEQ ID NO: 2) |
| 19-77 heavy chain | ELIISRNY (SEQ ID NO: 3) |
| CDRH1 | |
| 19-77 heavy chain | IYPGGSS (SEQ ID NO: 4) |
| CDRH2 | |
| 19-77 heavy chain | VRDAPSESD (SEQ ID NO: 5) |
| CDRH3 | |
| 19-77 light chain | QNIGHF (SEQ ID NO: 6) |
| CDRL1 | |
| 19-77 light chain | DAS (SEQ ID NO: 7) |
| CDRL2 | |
| 19-77 light chain | QERSDWPRGT (SEQ ID NO: 8) |
| CDRL3 | |

In some embodiments, the amino acid sequence of the antibody VH domain comprises SEQ ID NO: 1. In some embodiments, the amino acid sequence of the antibody heavy chain comprises a signal peptide immediately followed by SEQ ID NO: 1. In some embodiments, the signal peptide is cleaved during post-translational modifications that occur in vitro or in vivo. In some embodiments, a nucleic acid sequence encoding the amino acid sequence of the antibody heavy chain comprises a nucleic acid sequence encoding a signal peptide immediately followed by a nucleic acid sequence encoding SEQ ID NO: 1.

In some embodiments, the antibody comprises a polypeptide heavy chain comprising a variable heavy chain (VH) domain comprising SEQ ID NO: 1 followed by a constant region comprising a CH1 domain, a hinge domain, a Fc domain (CH2-CH3). In some embodiments the constant region comprises a CH1 domain, a hinge domain, and a Fc domain (CH2-CH3) of a human IgG1, IgG2, IgG3, or IgG4 constant region. In some embodiments the constant region comprises a CH1 domain, a hinge domain, and a Fc domain (CH2-CH3) of a human IgG1 constant region.

In some embodiments, the amino acid sequence of the antibody VL domain comprises SEQ ID NO: 2. In some embodiments, the amino acid sequence of the antibody light chain comprises a signal peptide immediately followed by SEQ ID NO: 2. In some embodiments, the signal peptide is cleaved during post-translational modifications that occur in vitro or in vivo. In some embodiments, a nucleic acid sequence encoding the amino acid sequence of the antibody light chain comprises a nucleic acid sequence encoding a signal peptide immediately followed by a nucleic acid sequence encoding SEQ ID NO: 2.

In some embodiments, the antibody comprises a polypeptide light chain comprising a variable light chain (VL) domain comprising SEQ ID NO: 2 followed by a constant region comprising a CL domain. In some embodiments the constant region comprises a CL domain of a human Ig kappa chain or Ig lambda chain. In some embodiments the constant region comprises a CL of a human Ig kappa chain.

In some embodiments, the monoclonal antibody comprises a first and third chain each comprising SEQ ID NO: 1 and a second and fourth chains each comprising SEQ ID NO: 2. In some embodiments, the monoclonal antibody comprises a first and third chain each comprising SEQ ID NO: 1 followed by a constant region comprising a CH1 domain, a hinge domain, a Fc domain (CH2-CH3) and a second and fourth chains each comprising SEQ ID NO: 2 followed by a constant region comprising a CL domain, wherein the constant region of the first and third chain comprises a CH1 domain, a hinge domain, and a Fc domain (CH2-CH3) of a human IgG1, IgG2, IgG3, or IgG4 constant region and the constant region of the second and fourth chain comprises a CL domain of a human Ig kappa chain or Ig lambda chain. In some embodiments, the first and second chains are linked by one or more covalent disulfide bonds. In some embodiments, the third and fourth chains are linked by one or more covalent disulfide bonds. In some embodiments, the first and third chains are linked by one or more disulfide bonds.

In some embodiments, the antibody heavy chain comprises a VH domain comprising an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the antibody light chain comprises a VL domain comprising an amino acid sequence 80, 81, 82, 83, 84, 85, 86, 87, 88, 89, 90, 91, 92, 93, 94, 95, 96, 97, 98 or 99% identical to SEQ ID NO: 2.

In some embodiments, the antibody heavy chain comprises a VH domain comprising CDR sequences SEQ ID NO: 3 (CDR-H1), SEQ ID NO: 4 (CDR-H2), and SEQ ID NO: 5 (CDR-H3). In some embodiments, the VH domain comprises FRs of a human IGHV1 gene. In some embodiments, the antibody light chain comprises a VL domain comprising CDR sequences SEQ ID NO: 6 (CDR-L1), SEQ ID NO: 7 (CDR-L2), and SEQ ID NO: 8 (CDR-L3).

In some embodiments, one or more amino acids of an amino acid sequence encoding SEQ ID NO: 1 or SEQ ID NO: 2 is substituted. In some embodiments, one or more amino acids of an amino acid sequence encoding one or more CDRs of SEQ ID NO: 1 or SEQ ID NO: 2 is substituted. In some embodiments, one or more amino acids of an amino acid sequence encoding one or more variable heavy chain CDRs of SEQ ID NO: 1 is substituted. In some embodiments, one or more amino acids of an amino acid sequence encoding one or more variable light chain CDRs of SEQ ID NO: 2 is substituted.

In some embodiments, the substitution occurs within framework region 3 of the heavy chain. Amino acids from VH and VL domains of immunoglobulins can be designated by the position of an amino acid in the chain. Kabat described numerous amino acid sequences for antibodies, identified an amino acid consensus sequence for each subgroup, and assigned a residue number to each amino acid (Kabat et al., SEQUENCES OF PROTEINS OF IMMUNOLOGICAL INTEREST, 5th Ed. Public Health Service, NH1, MD (1991), the contents of which is hereby incorporated by reference in its entirety). Kabat's numbering scheme is extendible to antibodies not included in his compendium by aligning the antibody in question with one of the consensus sequences in Kabat by reference to conserved amino acids. This method for assigning residue numbers has become standard in the field and readily identifies amino acids at equivalent positions in different antibodies. Thus, in some embodiments, the substitution occurs at residue 71 within framework region 3 of the heavy chain, using Kabat numbering. In some embodiments, the substitution occurs at residue 71 of SEQ ID NO:1. In some embodiments, the substitution is R71A. In some embodiments, the substitution is R71C. In some embodiments, the substitution is R71D. In some embodiments, the substitution is R71E. In some embodiments, the substitution is R71F. In some embodiments, the substitution is R71G. In some embodiments, the substitution is R71H. In some embodiments, the substitution is R71I. In some embodiments, the substitution is R71K. In some embodiments, the substitution is R71L. In some embodiments, the substitution is R71M. In some embodiments, the substitution is R71N. In some embodiments, the substitution is R71P. In some embodiments, the substitution is R71Q. In some embodiments, the substitution is R71S. In some embodiments, the substitution is R71T. In some embodiments, the substitution is R71V. In some embodiments, the substitution is R71W. In some embodiments, the substitution is R71Y.

In some embodiments, the antibody heavy chain comprises a VH comprising a heavy chain CDR1 having a sequence identity of at least 90%, 91%, 92%, 93%, 94%, 95%, 96%, 97%, 98%, or 99% identity to SEQ ID NO: 3, a heavy chain CDR2 having a sequence identity of at least 90%, 91%, 92%, 93%, 94%, 95%, 96%, 97%, 98%, or 99% identity to SEQ ID NO: 4, a heavy chain CDR3 having a sequence identity of at least 90%, 91%, 92%, 93%, 94%, 95%, 96%, 97%, 98%, or 99% identity to SEQ ID NO: 5 and the antibody light chain comprises a VL comprising a light chain CDR1 having a sequence identity of at least 90%, 91%, 92%, 93%, 94%, 95%, 96%, 97%, 98%, or 99% identity to SEQ ID NO: 6, a light chain CDR2 having a sequence identity of at least 90%, 91%, 92%, 93%, 94%, 95%, 96%, 97%, 98%, or 99% identity to SEQ ID NO: 7, and a light chain CDR3 having a sequence identity of at least 90%, 91%, 92%, 93%, 94%, 95%, 96%, 97%, 98%, or 99% identity to SEQ ID NO: 8.

In some embodiments, neutralization activity of the antibodies disclosed herein can be measured. In some embodiments, neutralization activity is measured by infecting cells with viral particles and an antibody of interest is added to the culture and determining the number of cells become infected. In some embodiments, the viral particles are chimeric viral particles known as pseudoviruses. In some embodiments, the pseudovirus is a vesicular stomatitis virus (VSV) particle. In some embodiments, the native glycoprotein (G-protein) in the VSV-particle which allows for viral entry into the cell is deleted (VSVΔG). In some embodiments, the VSVΔG is genetically engineered to express another G-protein (VSV-G*ΔG). In some embodiments, the expressed G-protein of the VSV-G*ΔG is the spike protein of a sarbecovirus. In some embodiments, the VSV-G*ΔG further packages expression cassettes for luciferase. In some embodiments, neutralization activity of an antibody of interest can be measured by measuring the luciferase expression of VSV-G*ΔG infected cells. In some embodiments, the neutralization activity is measured by determining the concentration of the antibody of interest needed to reach 50% of the fluorescence of cells infected VSV-G*ΔG alone (IC₅₀).

In some embodiments, the antibodies disclosed herein neutralize one or more sarbecoviruses. In some embodiments, the antibodies disclosed herein bind the spike protein one or more sarbecoviruses. In some embodiments the sarbecovirus is SARS-CoV. In some embodiments the sarbecovirus is SARS-CoV-2. In some embodiments the sarbecovirus is SARS-CoV-2-D614G. In some embodiments the sarbecovirus is SARS-CoV-2 B.1.1.7 (alpha) variant. In some embodiments the sarbecovirus is SARS-CoV-2 B.1.351 (beta) variant. In some embodiments the sarbecovirus is SARS-CoV-2 P.1 (gamma) variant. In some embodiments the sarbecovirus is SARS-CoV-2 B.1.617.2 (delta) variant. In some embodiments the sarbecovirus is SARS-CoV-2 BA.1 variant. In some embodiments the sarbecovirus is SARS-CoV-2 BA.2 variant. In some embodiments the sarbecovirus is SARS-CoV-2 BA.5 variant. In some embodiments the sarbecovirus is SARS-CoV-2 In some embodiments the sarbecovirus is SARS-CoV-2 BA.2.75 variant. In some embodiments the sarbecovirus is SARS-CoV-2 BQ.1.1. In some embodiments the sarbecovirus is SARS-CoV-2 XBB.1.5 variant. In some embodiments the sarbecovirus is SARS-CoV-2 EG.5.1 variant. In some embodiments the sarbecovirus is SARS-CoV-2 BA.2.86 variant. In some embodiments the sarbecovirus is SARS-CoV-2 JN.1 variant. In some embodiments the sarbecovirus is SARS-CoV-2 FL.1.5.1 variant. In some embodiments the sarbecovirus is SARS-CoV-2 HK.3. In some embodiments the sarbecovirus is SARS-CoV-2 JF.1 variant. In some embodiments the sarbecovirus is SARS-CoV-2 JD.1.1.

### Spike Protein

One of the key characteristics of coronaviruses is the presence of spike proteins on the surface of the virus that allow these viruses to enter a host cells. Spike (S) proteins of coronaviruses include two functional subunits: the N-terminal S1 subunit, which forms the globular head of the S protein, and the C-terminal S2 subunit that forms the stalk of the protein. The S2 subunit is embedded into the viral envelope. The S1 subunit includes an N-terminal domain and a receptor-binding domain (RBD) (https://www.ncbi.nlm.nih.gov/protein/7F5H_B). The S2 subunit includes the fusion peptide (FP), heptapeptide repeat sequence 1 (HR1), HR2, TM domain, and cytoplasm domain.

### Pharmaceutical Compositions

In some embodiments, the subject matter disclosed herein provides a pharmaceutical composition comprising any one of the antibodies disclosed herein or a combination thereof. In some embodiments, the pharmaceutical composition comprises a therapeutically effective amount of any one of the antibodies disclosed herein or a combination thereof. In some embodiments, the pharmaceutical composition comprises the 19-77 antibody disclosed herein. In some embodiments the pharmaceutical composition comprises the 19-77 antibody disclosed herein in combination with any other antibody disclosed herein, In some embodiments, the pharmaceutical composition disclosed herein is administered with another suitable anti-viral therapy. In some embodiments, the pharmaceutical composition disclosed herein is administered with another anti-sarbecovirus antibody. In some embodiments, the pharmaceutical composition disclosed herein further comprises one or more pharmaceutically-acceptable diluents, one or more pharmaceutically-acceptable carriers, or one or more pharmaceutically-acceptable excipients.

In some embodiments, the subject matter disclosed herein provides a polynucleotide encoding any of the antibodies disclosed herein. In some embodiments, the subject matter disclosed herein provides a genetically engineered cell comprising any of the anti-sarbecoviruses antibodies disclosed herein. In some embodiments, the subject matter disclosed herein provides a genetically engineered cell comprising a polynucleotide encoding the any of the antibodies disclosed herein.

In certain aspects, the subject matter disclosed herein provides pharmaceutical compositions comprising the above-described antibodies (or one or more polynucleotides encoding one or more antibodies). In some embodiments, the subject matter described herein relates to a pharmaceutical composition comprising an effective amount of the antibodies (or one or more polynucleotides encoding one or more antibodies) described herein and a pharmaceutically-acceptable diluent, carrier or excipient. In certain embodiments, the antibodies are conjugated with other molecules to increase their effectiveness as is known by those practiced in the art.

As used herein, “pharmaceutical composition” means a therapeutically effective formulation according to the invention. A “therapeutically effective amount”, or “effective amount”, or “therapeutically effective”, as used herein, refers to that amount which provides a therapeutic effect for a given condition and administration regimen. A therapeutically effective amount can be determined by a skilled person based on patient characteristics, such as age, weight, sex, condition, complications, other diseases, etc., as is well known in the art.

In some embodiments, the pharmaceutical compositions described herein can be administered as solid compositions. In some embodiments, the solid compositions comprise excipients including but not limited to lactose, starch, cellulose, milk sugar or high molecular weight polyethylene glycols. In some embodiments, the pharmaceutical compositions described herein can be administered as aqueous suspensions and/or elixirs. In some embodiments, the pharmaceutical compositions described herein may be combined with various sweetening or flavouring agents, coloring matter or dyes, with emulsifying and/or suspending agents and with diluents such as water, ethanol, propylene glycol and glycerin, and combinations thereof.

In some embodiments, the pharmaceutical compositions described herein can be administered parenterally, for example, intravenously, intra-arterially, intraperitoneally, intra-thecally, intraventricularly, intrasternally, intracranially, intra-muscularly or subcutaneously, or they may be administered by infusion techniques. In some embodiments, the pharmaceutical compositions described herein are administered in the form of a sterile aqueous solution which may contain other substances, for example, enough salts or glucose to make the solution isotonic with blood. The preparation of suitable parenteral formulations under sterile conditions is readily accomplished by standard pharmaceutical techniques well-known to those skilled in the art.

In some embodiments, pharmaceutical compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The pharmaceutical compositions can be presented in unit-dose or multi-dose containers. The pharmaceutical compositions can be sealed ampoules or vials. The pharmaceutical compositions can be stored in a freeze-dried (lyophilised) condition requiring only the addition of the sterile liquid carrier, such as water for injections, immediately prior to use.

The compounds and pharmaceutical compositions of the present invention can be employed in combination therapies, that is, the compounds and pharmaceutical compositions can be administered concurrently with, prior to, or subsequent to, one or more other desired therapeutics or medical procedures (e.g., antibodies can be used in combination treatment with another treatment). The particular combination of therapies (therapeutics or procedures) to employ in a combination regimen will consider compatibility of the desired therapeutics and/or procedures and the desired therapeutic effect to be achieved. It will also be appreciated that the therapies employed may achieve a desired effect for the same disorder (for example, the compound of the present invention may be administered concurrently with another therapeutic or prophylactic).

In some embodiments, the subject matter disclosed herein also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

In certain aspects, the subject matter disclosed herein provides a composition comprising: a first vector comprising a polynucleotide sequence encoding the first polypeptide chain of any of the monoclonal antibodies disclosed herein; and a second vector comprising a polynucleotide sequence encoding the second polypeptide chain of any of the monoclonal antibodies disclosed herein.

Compositions can be formulated with appropriate carriers and adjuvants using techniques to yield compositions suitable for prophylaxis or treatment. The compositions can include an adjuvant, such as, for example but not limited to, alum, poly IC, MF-59, squalene-based adjuvants, or liposomal based adjuvants suitable for prophylaxis or treatment.

In some embodiments, the antibodies described herein are encoded by nucleic acids which are prepared in a mRNA-LNP or a DNA-LNP formulation for administration to a subject.

### Antibody Production

The antibodies disclosed herein can be produced by any method known in the art. In some embodiments, the antibodies disclosed herein are produced by culturing a cell transfected or transformed with a vector comprising nucleic acid sequences encoding an antibody described herein and isolating the antibody.

In some embodiments, antibodies are synthesized by methods which results in antibodies that are not contaminated by other immunoglobulins. The monoclonal antibodies to be used in accordance with the present invention may be made by a variety of techniques known in the art, including, for example, the hybridoma method, recombinant DNA methods, phage-display technologies, B-cell discovery methods, and technologies for producing human or human-like antibodies in animals that have parts or all of the human immunoglobulin loci or genes encoding human immunoglobulin sequences.

The polynucleotide encoding the antibody may be modified, for example, by substituting the coding sequence for human heavy- and light-chain constant domains. The polynucleotide may also be modified by covalently joining to the immunoglobulin coding sequence all or part of the coding sequence for a non-immunoglobulin polypeptide. Such non-immunoglobulin polypeptides can be substituted for the constant domains of an antibody. The monoclonal antibodies described herein may by monovalent, the preparation of which is well known in the art. For example, one method involves recombinant expression of an immunoglobulin light chain and a modified heavy chain. The heavy chain is generally truncated at any point in the Fc domain so as to prevent heavy chain crosslinking. Alternatively, the relevant cysteine residues may be substituted with another amino acid residue or are deleted so as to prevent crosslinking. In vitro methods are also suitable for preparing monovalent antibodies. Antibiotic ingestion to produce fragments, particularly Fab fragments, can be accomplished using routine techniques known in the art. Chimeric or hybrid antibodies also may be prepared in vitro using known methods in synthetic protein chemistry, including those involving crosslinking agents.

Various expression systems for producing antibodies are known in the art, and include, prokaryotic (e.g., bacteria), plant, insect, yeast, and mammalian expression systems. Suitable cell lines, can be transformed, transduced, or transfected with nucleic acids containing coding sequences for antibodies or portions of antibodies disclosed herein in order to produce the antibody of interest. Expression vectors containing such nucleic acid sequences, which can be linked to at least one regulatory sequence in a manner that allows expression of the nucleotide sequence in a host cell, can be introduced via methods known in the art. Practitioners in the art understand that designing an expression vector can depend on factors, such as the choice of host cell to be transfected and/or the type and/or amount of desired protein to be expressed. Enhancer regions, which are those sequences found upstream or downstream of the promoter region in non-coding DNA regions, are also known in the art to be important in optimizing expression. If needed, origins of replication from viral sources can be employed, such as if a prokaryotic host is utilized for introduction of plasmid DNA. However, in eukaryotic organisms, chromosome integration is a common mechanism for DNA replication. For stable transfection of mammalian cells, a small fraction of cells can integrate introduced DNA into their genomes. The expression vector and transfection method utilized can be factors that contribute to a successful integration event. For stable amplification and expression of a desired protein, a vector containing DNA encoding a protein of interest (e.g., antibodies and fragments thereof) is stably integrated into the genome of eukaryotic cells (for example mammalian cells), resulting in the stable expression of transfected genes. A gene that encodes a selectable marker (for example, resistance to antibiotics or drugs) can be introduced into host cells along with the gene of interest in order to identify and select clones that stably express a gene encoding a protein of interest. Cells containing the gene of interest can be identified by drug selection wherein cells that have incorporated the selectable marker gene will survive in the presence of the drug. Cells that have not incorporated the gene for the selectable marker die. Surviving cells can then be screened for the production of the desired antibody molecule.

Various culturing parameters can be used to culture the host cell. Appropriate culture conditions for mammalian cells are well known in the art or can be determined by the skilled artisan (see, for example, Animal Cell Culture: A Practical Approach 2^{nd} Ed., Rickwood, D. and Hames, B. D., eds. (Oxford University Press: New York, 1992)). Cell culturing conditions can vary according to the type of host cell selected. Commercially available media can be utilized.

Antibodies disclosed herein can be purified from any human or non-human cell that expresses the antibody, including those that have been transfected with expression constructs that express the antibody or fragments thereof. For antibody recovery, isolation and/or purification, the cell culture medium or cell lysate is centrifuged to remove particulate cells and cell debris. The desired antibody molecule is isolated or purified away from contaminating soluble proteins and polypeptides by suitable purification techniques. Non-limiting purification methods for proteins/antibodies include: size exclusion chromatography, affinity chromatography, ion exchange chromatography, ethanol precipitation; reverse phase HPLC; chromatography on a resin, such as silica, or cation exchange resin, e.g., DEAE; chromatofocusing; SDS-PAGE; ammonium sulfate precipitation; gel filtration using, e.g., Sephadex G-75, Sepharose; protein A sepharose chromatography for removal of immunoglobulin contaminants; and the like. Other additives, such as protease inhibitors (e.g., PMSF or proteinase K) can be used to inhibit proteolytic degradation during purification. Purification procedures that can select for carbohydrates can also be used, e.g., ion-exchange soft gel chromatography, or HPLC using cation- or anion-exchange resins, in which the more acidic fraction(s) is/are collected.

### Methods of Treatment

In one embodiment, the subject matter disclosed herein relates to a preventive medical treatment started after following diagnosis of a disease (e.g., viral infection) in order to prevent the disease from worsening or curing the disease. In one embodiment, the subject matter disclosed herein relates to prophylaxis of subjects who are at risk for moderate or severe disease associated with a viral infection or have previously been diagnosed with another disease, such as a viral infection. In one embodiment, the subjects can be administered the pharmaceutical composition described herein. The invention contemplates using any of the antibodies produced by the systems and methods described herein. In one embodiment, the compositions described herein can be administered subcutaneously via syringe or any other suitable method known in the art.

The antibodies disclosed herein, or pharmaceutical compositions may be administered to a cell, mammal, or human by any suitable means. In some embodiments, one or more antibodies disclosed herein are prepared in a cocktail of DNA-encoding antibodies or mRNA-encoding antibodies and delivered to a subject for in vivo expression of the encoded antibodies.

As will be readily apparent to one skilled in the art, the effective in vivo dose to be administered and the particular mode of administration will vary depending upon the age, weight and species treated, and the specific use for which the compound or combination of compounds disclosed herein are employed. The determination of effective dose levels, that is the dose levels necessary to achieve the desired result, can be accomplished by one skilled in the art using routine pharmacological methods. Typically, human clinical applications of products are commenced at lower dose levels, with dose level being increased until the desired effect is achieved. Alternatively, acceptable in vitro studies can be used to establish useful doses and routes of administration of the compositions identified by the present methods using established pharmacological methods. Effective animal doses from in vivo studies can be converted to appropriate human doses using conversion methods known in the art.

In certain aspects, the subject matter disclosed herein provides a method of treating or preventing sarbecovirus in a subject in need thereof, the method comprising administering to the subject an effective amount of any one of the monoclonal antibodies disclosed herein or any one of the pharmaceutical compositions disclosed herein.

### Kits of the Invention

In one embodiment, the subject matter disclosed herein relates to a kit for generating an anti-serberovirus antibody comprising an antibody composition of the present invention and instructions for use. In one embodiment, the subject matter disclosed herein relates to a kit for generating an anti-serberovirus antibody comprising one or more vectors comprising a polynucleotide sequence of any of the anti-serberovirus antibodies described above. The kit can further include at least one additional reagent or one or more antibodies of the present invention. The kit usually has a label indicating the intended use of the kit contents. The term label includes all documents and is attached to the kit or with the kit, or otherwise attached to the kit.

### Non-Limiting Embodiments of the Subject Matter

In certain aspects, the subject matter described herein provides A monoclonal antibody or an antigen-binding fragment thereof comprising (i) a heavy chain variable domain (VH) and (ii) a light chain variable domain (VL); wherein the monoclonal antibody or antigen binding fragment thereof binds a portion of a receptor-binding domain (RBD) on a spike protein of a sarbecovirus.

In some embodiments, the VH domain comprises a heavy chain CDR1 with at least 85%, 90%, 95%, or 99% identity to SEQ ID NO: 3, a heavy chain CDR2 with at least 85%, 90%, 95%, or 99% identity to SEQ ID NO: 4, and a heavy chain CDR3 with at least 85%, 90%, 95%, or 99% identity to SEQ ID NO: 5. In some embodiments, the VH domain comprises a heavy chain CDR1 identical to SEQ ID NO: 3, a heavy chain CDR2 identical to SEQ ID NO: 4, and a heavy chain CDR3 identical to SEQ ID NO: 5. In some embodiments, the VL domain comprises a light chain CDR1 with at least 85%, 90%, 95%, or 99% identity to SEQ ID NO: 6, a light chain CDR2 with at least 85%, 90%, 95%, or 99% identity to SEQ ID NO: 7, and a light chain CDR3 with least 85%, 90%, 95%, or 99% identity to SEQ ID NO: 8. In some embodiments, the VL domain comprises a light chain CDR1 identical to SEQ ID NO: 6, a light chain CDR2 identical to SEQ ID NO: 7, and a light chain CDR3 identical to SEQ ID NO: 8. In some embodiments, the VH domain has at least 85%, 90%, 95%, or 99% identity to SEQ ID NO: 1. In some embodiments, the VH domain comprises or is SEQ ID NO: 1. In some embodiments, the VL domain has at least 85%, 90%, 95%, or 99% identity to SEQ ID NO: 2. In some embodiments, the VL domain comprises or is SEQ ID NO: 2.

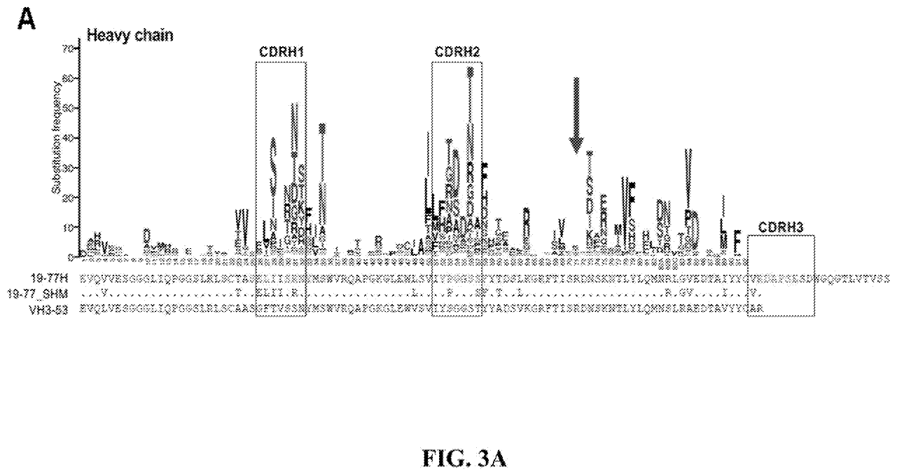

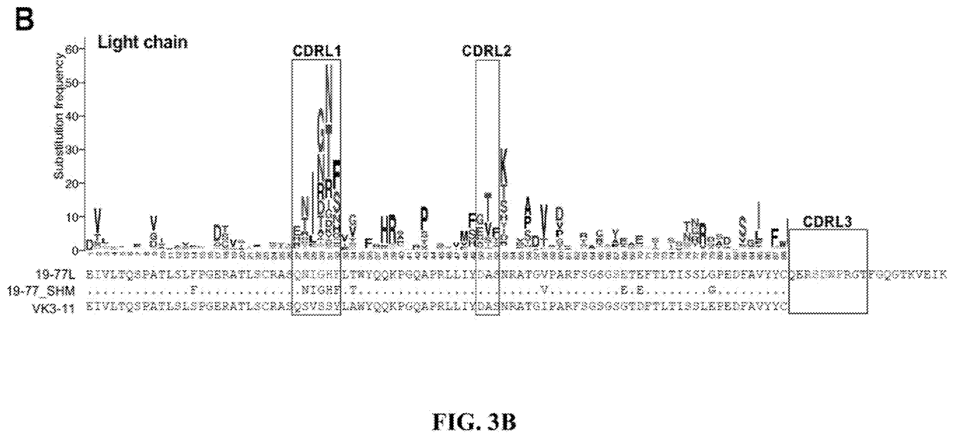

In some embodiments, the framework regions of the VL domain comprise at least 85%, 90%, 95%, or 99% identity to the framework regions of SEQ ID NO: 2. In some embodiments, the framework regions of the VH domain comprise framework regions that are derived from IGHV3-53 of FIG. 3A. In some embodiments, the VL domain comprise framework regions that are derived from IGKV3-11 of FIG. 3B. IN some embodiments, the framework regions of the VH domain comprise framework regions that are at least 85%, 90%, 95%, or 99% identical to the framework regions of IGHV3-53 of FIG. 3A. In some embodiments, the framework regions of the VL domain comprise framework regions that are at least 85%, 90%, 95%, or 99% identical to the framework regions of FIG. 3B. In some embodiments, the VH domain comprises at least 85%, 90%, 95%, or 99% identity to SEQ ID NO: 1. In some embodiments, the VH domain comprises SEQ ID NO: 1. In some embodiment, the VH domain consists of SEQ ID NO: 1. In some embodiments, the VL domain comprises at least 85%, 90%, 95%, or 99% identity to SEQ ID NO: 2. In some embodiments, the VL domain comprises SEQ ID NO: 2. In some embodiments, the VL domain consists of SEQ ID NO: 2.

In some embodiments, the Sarbecovirus is a SARS-CoV or SARS-CoV-2. In some embodiments, the Sarbecovirus is a SARS-CoV-2 variant. In some embodiments, the SARS-CoV-2 variant is D614G, B.1.1.7, B.1.351, P.1, B.1.617.2, BA.1, BA.2, BA.5, BA.2.75, CH.1.1, DV.7.1, XBC.1.6, BQ.1.1, XBB.1.5, XBB.1.16.6, XBB.2.3, EG.5.1, FL.1.5.1, JF.1, HV.1, HK.3, BA.2.87.1, BA.2.86, JN.1, JN.4, LB.1, KP.2, KP.3, KP.2.3, KP.3.1.1, XEC, GX-pangolin, RaTG13, BANAL52, BANAL236, or GD-pangolin.

In some embodiments, the VH domain comprises SEQ ID NO: 1 and the VL domain comprises SEQ ID NO:2. In some embodiments, the VH domain comprises at least 85%, 90%, 95%, or 99% identity to SEQ ID NO: 1 and the VL domain comprises at least 85%, 90%, 95%, or 99% identity to SEQ ID NO:2.

In some embodiments, the heavy chain CDR1, CDR2, and CDR3 of the monoclonal antibody of antigen-binding fragment thereof has an increased RMSF score as compared to heavy chain CDR1, CDR2, and CDR3 of a monoclonal antibody or fragment thereof comprising SEQ ID NO:1 and SEQ ID NO: 2. In some embodiments, the average RMSF score is above 1.0. In some embodiments, the average RMSF score is above 1.2.

In some embodiments, the VH domain comprises at least 85%, 90%, 95%, or 99% identity to SEQ ID NO: 1 and comprises an amino acid mutation at position 71. In some embodiments, the VH domain comprises SEQ ID NO:1 comprising an amino acid mutation at position 71. In some embodiments, the VH domain consists of SEQ ID NO: 1 with an amino acid mutation at position 71. In some embodiments, the VL domain comprises at least 85%, 90%, 95%, or 99% identity to SEQ ID NO: 2. In some embodiments, the VL domain comprises SEQ ID NO: 2. In some embodiments, the VL domain consists of SEQ ID NO: 2. In some embodiments, the amino acid mutation is R71A, R71C, R71D, R71E, R71F, R71G, R71H, R71I, R71K, R71L, R71M, R71N, R71P, R71Q, R71S, R71T, R71V, R71W, or R71Y. In some embodiments, the amino acid mutation is R71A. In some embodiments, the amino acid mutation is R71L. In some embodiments, the amino acid mutation is R71V.

In some embodiments, the monoclonal antibody or antigen-binding fragment thereof is a single chain antibody, Fab, Fab′, F(ab′)2, Fv or scFv. In some embodiments, the monoclonal antibody is an IgG type. In some embodiments, the monoclonal antibody is an IgA type.

In certain embodiments, the present application discloses a monoclonal antibody comprising a first arm comprising the VH domain and VL domain, wherein the VH and VL domain form a binding site that binds a portion of the RBD on the spike protein of a sarbecovirus; and a second arm comprising a second VH domain, identical to the VH domain of the first arm, and a second VL domain, identical to the VL domain of the first arm, wherein the second VH and second VL form a binding site that binds a portion of the RBD on the spike protein of a sarbecovirus. In some embodiments, the first arm and the second arm each further comprise a CH1 domain, a hinge domain, and a CL domain.

In certain embodiments, the present application discloses a monoclonal antibody comprising the VH domain of the first arm is encoded by a first polypeptide chain, the VL domain of the first arm is encoded by a second polypeptide chain, the second VH domain of the second arm is encoded by a third polypeptide chain, the second VL domain of the second arm is encoded by a fourth polypeptide chain. In some embodiments, the first polypeptide chain and the third polypeptide chain each further encode a hinge domain, a CH1 domain, the Fc domain, and wherein the second polypeptide chain and the fourth polypeptide chain each further encode a CL domain. In some embodiments, the first polypeptide chain and the third polypeptide chain comprise the same sequence, and wherein the second polypeptide chain and the fourth polypeptide chain comprise the same sequence. In some embodiments, the first polypeptide chain and the second polypeptide chain are linked by one or more covalent disulfide bonds and the third polypeptide chain and the fourth polypeptide chain are linked by one or more covalent disulfide bonds, and the first polypeptide chain and the third polypeptide chain are linked by one or more covalent disulfide bonds. In some embodiments, the monoclonal antibody or antigen-binding fragment thereof comprises an engineered constant domain.

In certain aspects, the subject matter described herein provides a pharmaceutical composition comprising therapeutically effective amount of a monoclonal antibody or an antigen-binding fragment thereof, comprising (i) a heavy chain (HC) variable domain (VH) and (ii) a light chain (LC) variable domain (VL); wherein the antibody, or antigen binding fragment thereof, binds a spike protein on a sarbecovirus.

In certain aspects, the subject matter described herein provides a method of treating a Sarbecovirus infection in a subject in need thereof, the method comprising administering to the subject a pharmaceutical composition comprising a therapeutically effective amount of a monoclonal antibody or an antigen-binding fragment thereof, comprising (i) a heavy chain (HC) variable domain (VH) and (ii) a light chain (LC) variable domain (VL); wherein the antibody, or antigen binding fragment thereof, binds a spike protein on a sarbecovirus.

In certain embodiments, the present application provides a means for binding a portion of the RBD on a spike protein of a sarbecovirus. In some embodiments, the means comprises any of the monoclonal antibody or antigen binding fragment thereof described above.

In certain aspects, the present application provides one or more vectors comprising a first vector comprising a nucleotide sequence encoding any of the VH chains described above a second vector comprising a nucleotide sequence encoding any of the VL chains described above. In some embodiments, the first and second vectors are the same vector. In some embodiments, the first and second vectors are two different vectors.

In certain aspects, the present application provides one or more host cells comprising
a first vector comprising a polynucleotide sequence encoding any of the VH chains described above and a second vector comprising a polynucleotide sequence encoding any of the VH chains described above. In some embodiments, the first vector and the second vector are the same vector. In some embodiments, the first vector and the second vector are two different vectors.

In some embodiments, the VH domain comprises a heavy chain CDR1 with at least 85%, 90%, 95%, or 99% identity to SEQ ID NO: 3, a heavy chain CDR2 with at least 85%, 90%, 95%, or 99% identity to SEQ ID NO: 4, and a heavy chain CDR3 with at least 85%, 90%, 95%, or 99% identity to SEQ ID NO: 5. In some embodiments, the VH domain comprises a heavy chain CDR1 identical to SEQ ID NO: 3, a heavy chain CDR2 identical to SEQ ID NO: 4, and a heavy chain CDR3 identical to SEQ ID NO: 5. In some embodiments, the VL domain with a light chain CDR1 comprising at least 85%, 90%, 95%, or 99% identity to SEQ ID NO: 6, a light chain CDR2 with at least 85%, 90%, 95%, or 99% identity to SEQ ID NO: 7, and a light chain CDR3 with at least 85%, 90%, 95%, or 99% identity to SEQ ID NO: 8. In some embodiments, the VL domain comprises a light chain CDR1 identical to SEQ ID NO: 6, a light chain CDR2 identical to SEQ ID NO: 7, and a light chain CDR3 identical to SEQ ID NO: 8. In some embodiments, the VH domain has at least 85%, 90%, 95%, or 99% identity to SEQ ID NO: 1. In some embodiments, the VH domain comprises or is SEQ ID NO: 1. In some embodiments, the VL domain has at least 85%, 90%, 95%, or 99% identity to SEQ ID NO: 2. In some embodiments, the VL domain comprises or is SEQ ID NO: 2.

In some embodiments, the monoclonal antibody or antigen-binding fragment thereof is derived from a VH3-53 germline. In some embodiments, the monoclonal antibody or antigen-binding fragment thereof binds a receptor-binding domain (RBD) on the spike protein. In some embodiments, the monoclonal antibody or antigen-binding fragment thereof is isolated from a patient. In some embodiments, the patient is vaccinated against one or multiple Sarbecoviruses. In some embodiments, the patient has recovered from one or multiple Sarbecoviruses.

In some embodiments, the spike protein is from the Sarbecovirus SARS-CoV. In some embodiments, the spike protein is from the Sarbecovirus SARS-CoV-2. In some embodiments, the Sarbecovirus is a SARS-CoV-2 variant selected from SARS-CoV-2-D614G, B.1.1.7, B.1.351, P.1, B.1.617.2, BA.1, BA.2, BA.5, BA.2.75, BQ.1.1, XBB.1.5, EG.5.1, BA.2.86, JN.1, FL.1.5.1, HK.3, JF.1, JD.1.1. In some embodiments, the monoclonal antibody or antigen-binding fragment thereof has a R71X amino acid mutation selected from R71A, R71C, R71D, R71E, R71F, R71G, R71H, R71I, R71K, R71L, R71M, R71N, R71P, R71Q, R71S, R71T, R71V, R71W, and R71Y. In some embodiments, the mutation is R71A. In some embodiments, the mutation is R71L. In some embodiments, the mutation is R71V.

In some embodiments, the subject is an animal or a human. In some embodiments, the subject is a human subject. In some embodiments, the subject is a mammal. In some embodiments, the subject is a mammalian subject. In some embodiments, the animal is a mouse. In some embodiments, the animal is a rat.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

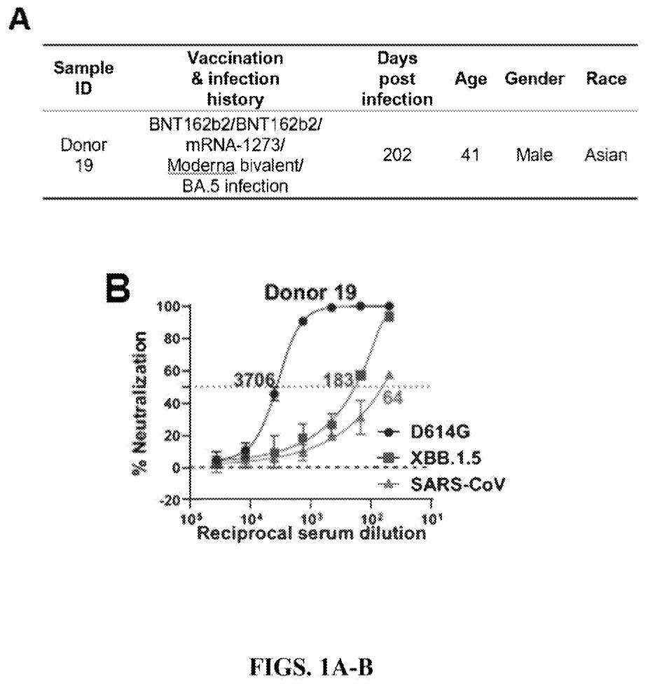

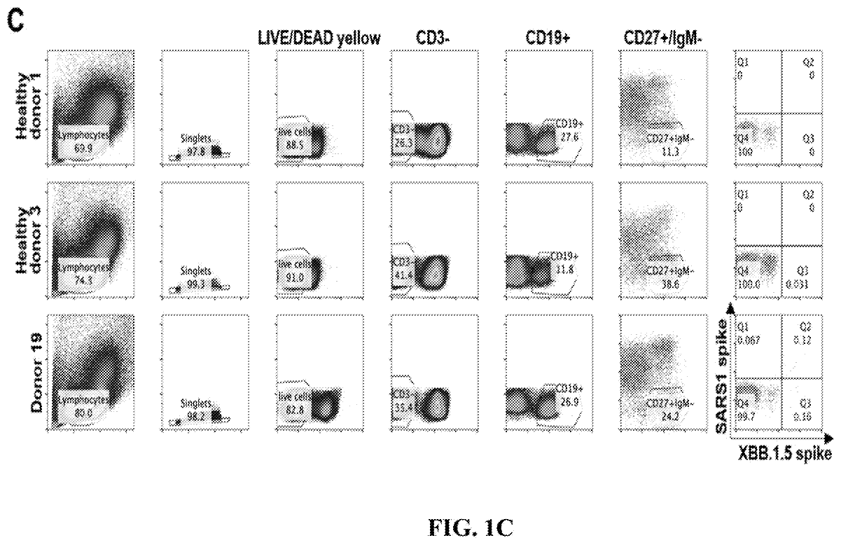

As shown in FIG. 1A-C, blood samples were collected from donor 19. XBB.1.5 and/or SARS-CoV specific B cells from the PBMC samples of this donor were isolated. The sorted B cells were subjected to 10× Genomics and then 102 pairs of antibody genes were synthesized.

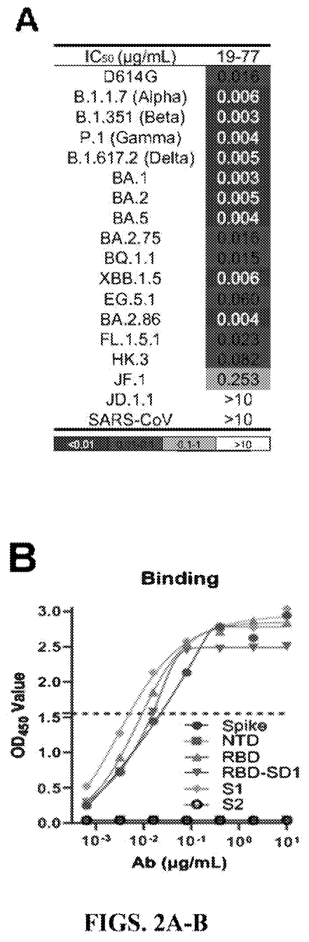

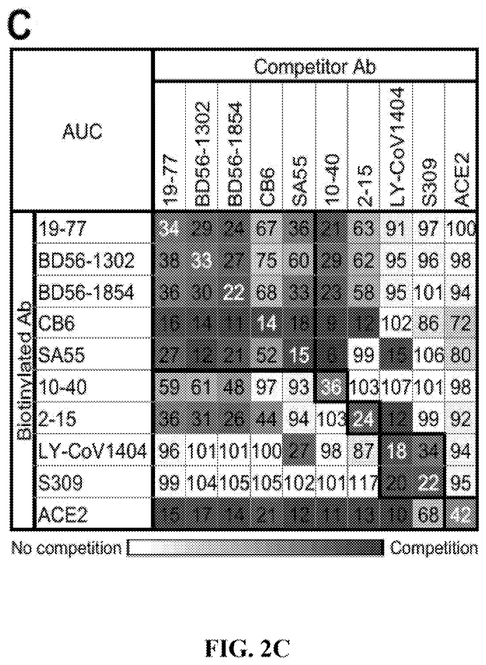

Among the 102 isolated antibodies from donor 19, one mAb shows good potency and breadth against the SAR-CoV-2 virus and its variants. This mAb is 19-77, as shown in FIGS. 2A-C. 19-77 mAb is impaired by JF.1 and knocked out by JD.1.1. ELISA data in FIG. 2B suggests that 19-77 is a RBD binder. Competition ELISA data in FIG. 2C further suggests that this antibody targets the class 1 epitope in the RBD, as it strongly competes with BD56-1302 and BD56-1854, which are RBD class 1 antibodies.

FIGS. 3A-B show genetic analyses that demonstrate that the 19-77 mAb heavy chain is from the VH3-53 family while the light chain comes from VK3-11 family. FIG. 3A shows SEQ ID NO 1 which encodes the heavy chain of mAb 19-77 (first line). FIG. 3B shows SEQ ID NO 2 which encodes the light chain of mAb 19-77 (first line).

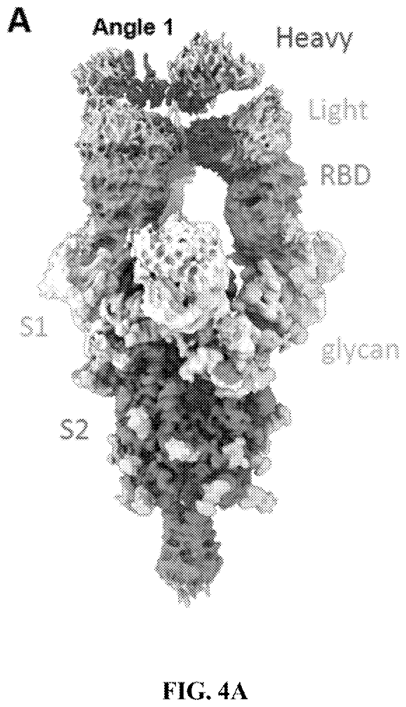

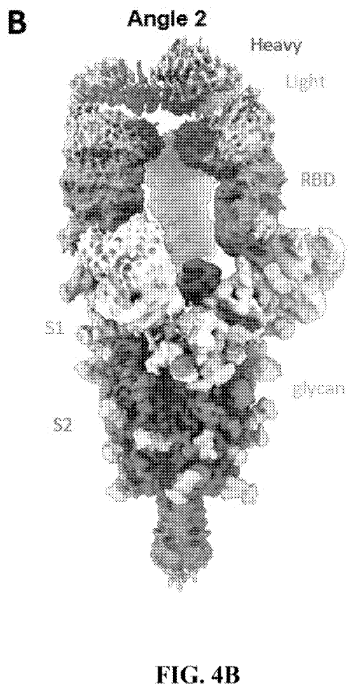

FIGS. 4A-B show global reconstruction of the mAb 19-77 with the D614G spike. FIG. 4A shows angle 1 of the reconstruction. FIG. 4B shows angle 2 of the reconstruction. Cryo-EM reconstruction of mAb 19-77 in complex with SARS-CoV-2 D614G spike at resolutions of 2.45 A°.

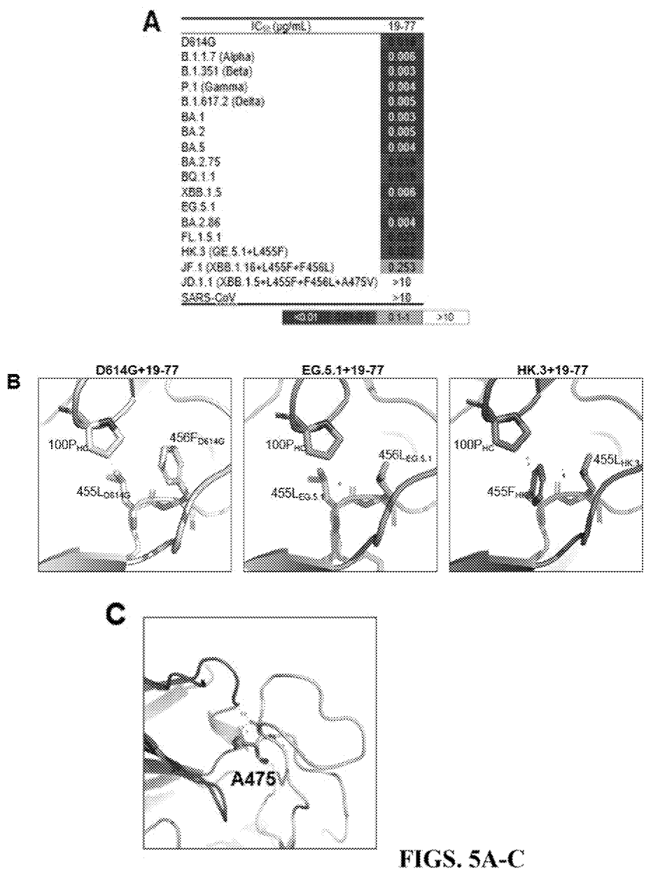

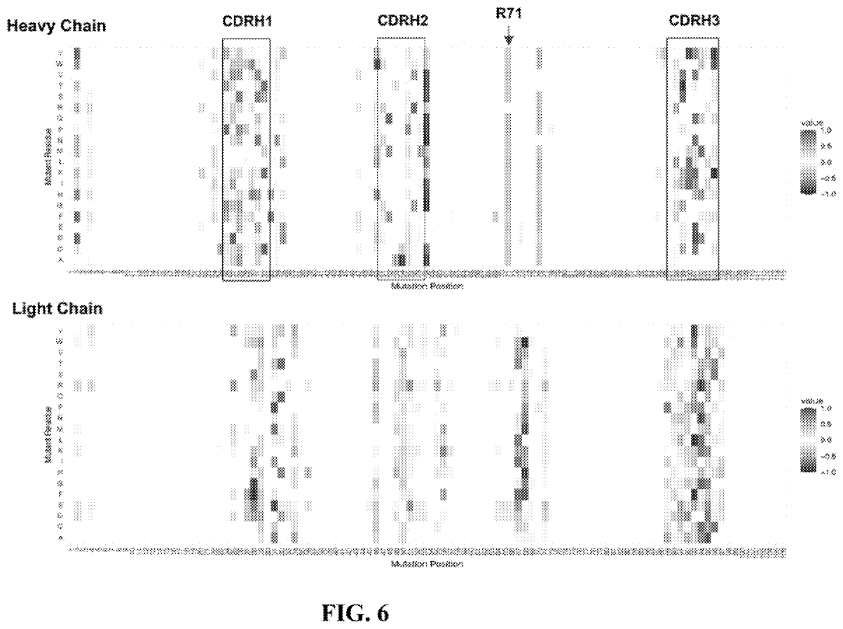

FIGS. 5A-C show mutations on the spike protein, which help the virus escape neutralization by 19-77 mAb. FIG. 6 shows In silico optimization of 19-77 in complex with HK.3 RBD.

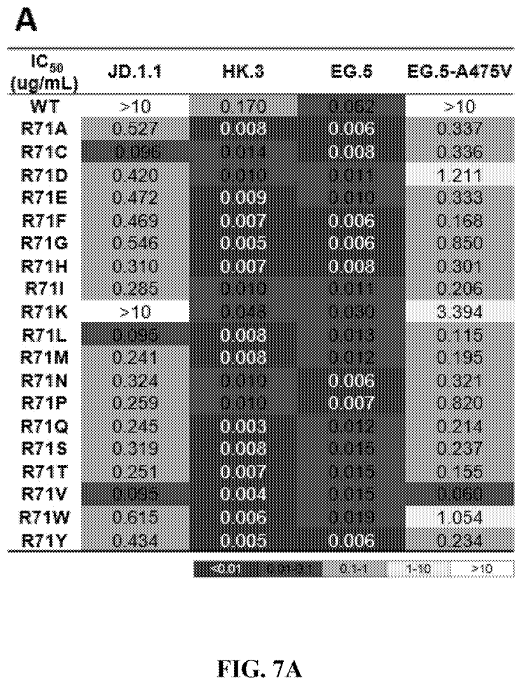

FIGS. 7A-B show neutralizing IC₅₀ activity of Ho71X mutants of the 19-77 mAb (19-77 carrying various amino acid substitutions at position 71 in the heavy chain). All the mutations (except for R71K) in the heavy chain of 19-77 help to rescue/sensitize the neutralization activity of 19-77 against the indicated viruses.

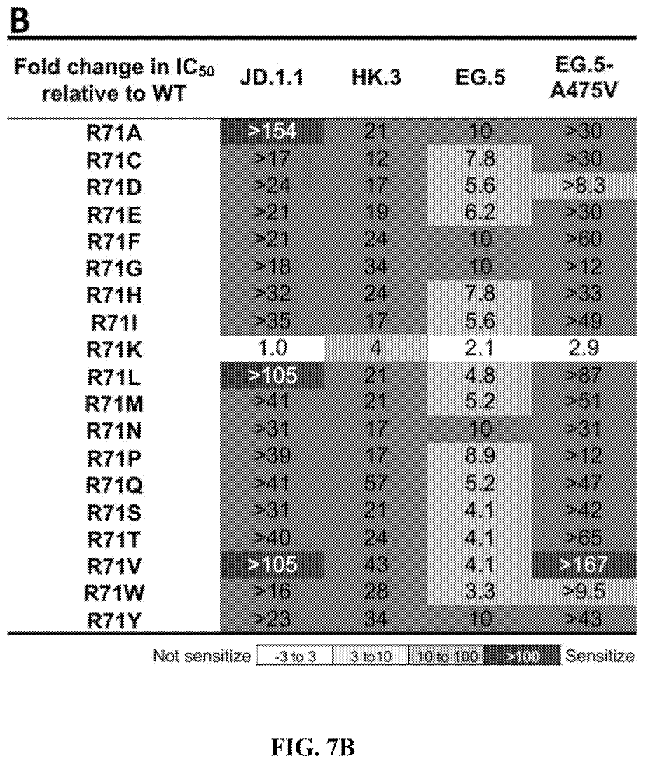

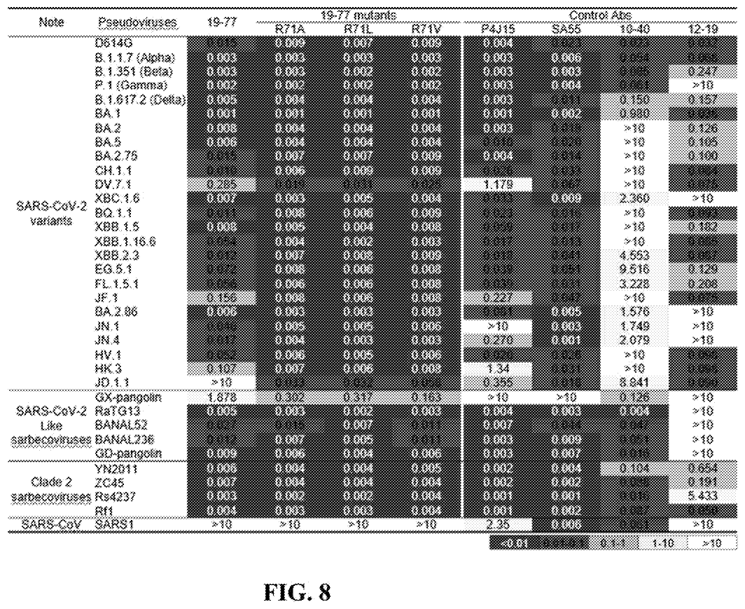

FIG. 8 shows neutralization IC₅₀ values of 19-77 mAb and 19-77 mutants against SARS-CoV-2 variants, SARS-CoV-2 like sarbecoviruses, clade 2 sarbecoviruses, and SARS-CoV, in comparison with that of other RBD-specific neutralizing antibodies including P4J15, SA55, 10-40, and 12-19. In comparison with other reported antibodies, like P4J15, SA55, 10-40, and 12-19, the 19-77 mutants (R71A/V/L) showed better potencies and breadth against these viruses, except for SARS-CoV.

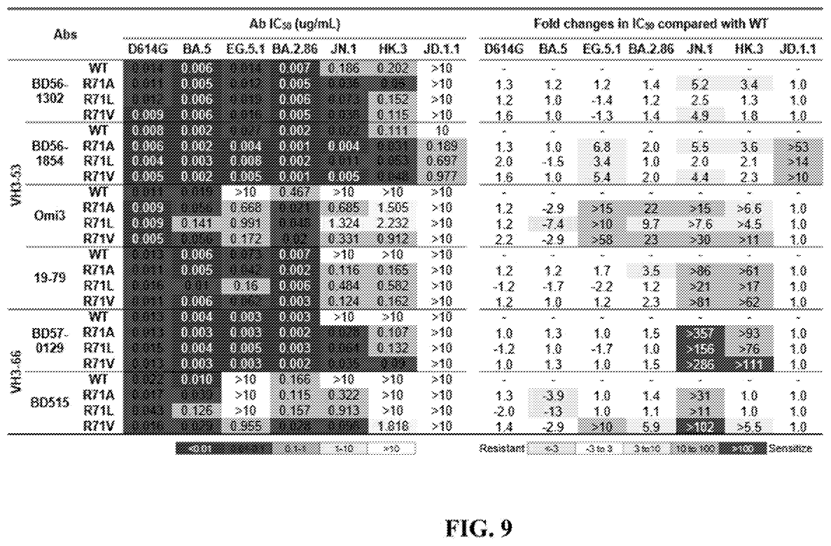

FIG. 9 shows that R71/A/L/V mutations rescue the neutralizing activities of other VH3-53 and VH3-66 antibodies against SARS-CoV-2 escaping variants.

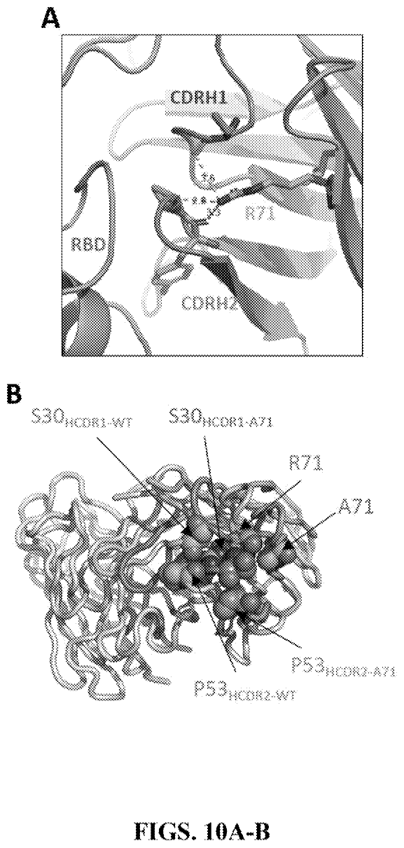

FIGS. 10A-B show the mechanism of antibody neutralization improvement. R71 contacts with the CDRH1 and CDRH2 of the heavy chain of 19-77 by hydrogen bonds and Ho71X mutations increase the flexibility of the CDRH1 and CDRH2 and therefore sensitize antibody neutralization. Structurally, in antibody 19-77, R71 forms hydrogen bonds with both the CDRH1 and CDRH2. For clarity in the visual representations, CDRH1 is marked in blue and CDRH2 in orange. R71 may constrain the movement of CDRH1 and CDRH2. To explore this, molecular dynamics simulations were constructed comparing the WT 19-77 mAb with its R71A mutant variant. The results suggest that the R71A mutation increases the flexibility of the 19-77 Fab region. This is evident in a video analysis, where the residues interacting with R71 in the WT exhibit less motion compared to those in the R71A variant.

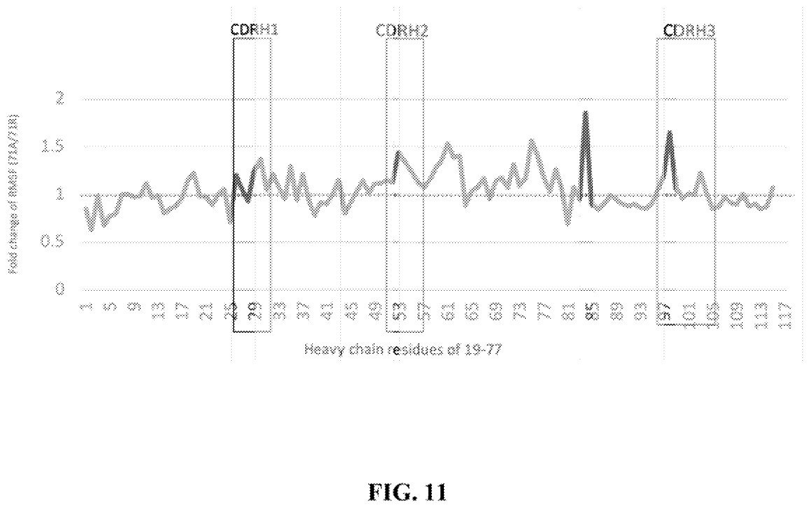

FIG. 11 shows comparison of root mean square fluctuation (RMSF) between the Fab regions of 19-77 and its mutant 19-77-R71A. R71A increases the flexibility of CDRH1, CDRH2 and CDRH3 of 19-77.

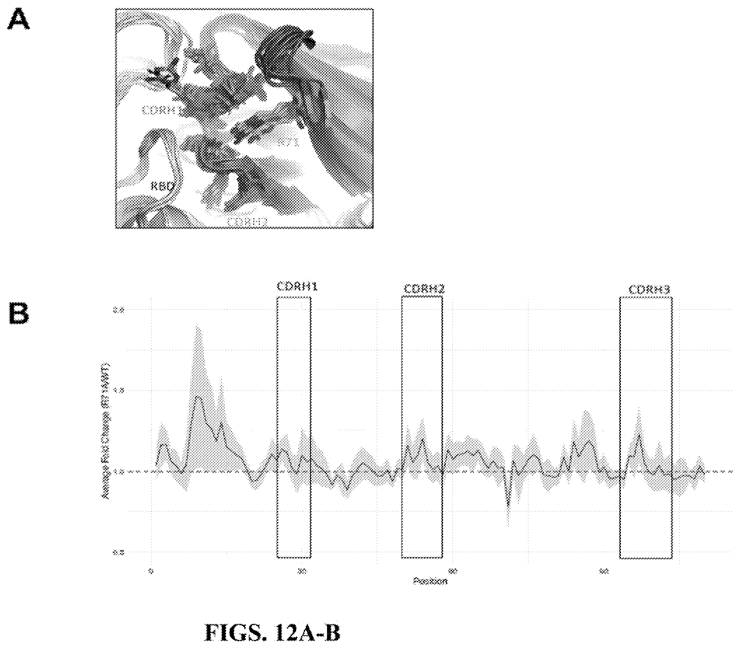

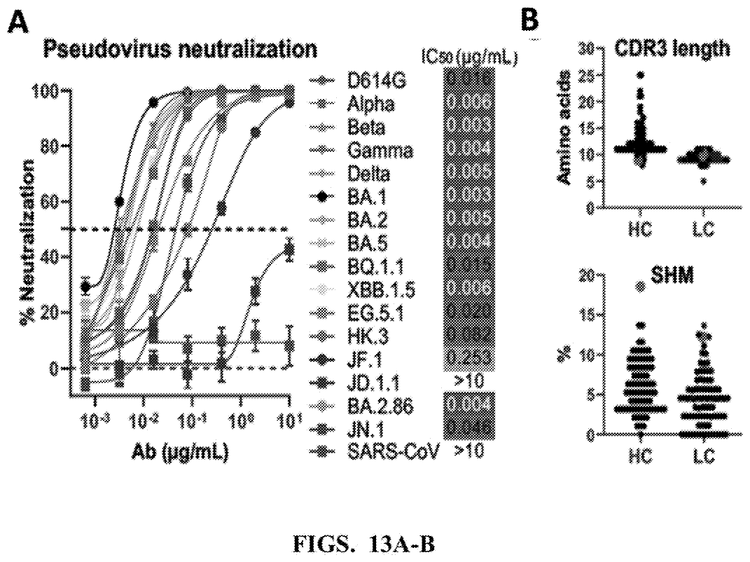

FIGS. 12A-B shows comparative analysis of VH3-53 class antibodies with R71A mutation. FIG. 12A shows structural alignment of 10 VH3-53 class antibodies, highlighting the location of residue R71 in relation to CDRH1 and CDRH2. FIG. 12B shows changes in RMSF in the heavy chains of V13-53 class antibodies. 10 VH3-53 antibodies were analyzed and all of them showed increased flexibility of CDRH1, CDRH2, and CDRH3 when R71 is mutated to A.

### Example 2

SARS-CoV-2 has largely evolved to resist antibody pressure, with each successive viral variant becoming more and more resistant to serum antibodies in the population. This evolution has rendered inactive all therapeutic monoclonal antibodies previously authorized, and it is now threatening the remaining clinical product for immunoprophylaxis against COVID-19. In some embodiments, the subject matter disclosed herein relates to the isolation of a human monoclonal antibody with a broad but incomplete SARS-CoV-2 neutralization profile, but structural analyses and mutational scanning led to the engineering of variants that resulted in greater antibody flexibility while binding to the viral spike. Three such optimized monoclonal antibodies neutralized all SARS-CoV-2 strains tested with much improved potency and breadth, including against subvariants KP.3.1.1 and XEC that are dominant today.

### Results

### Isolation and Characterization of mAb 19-77

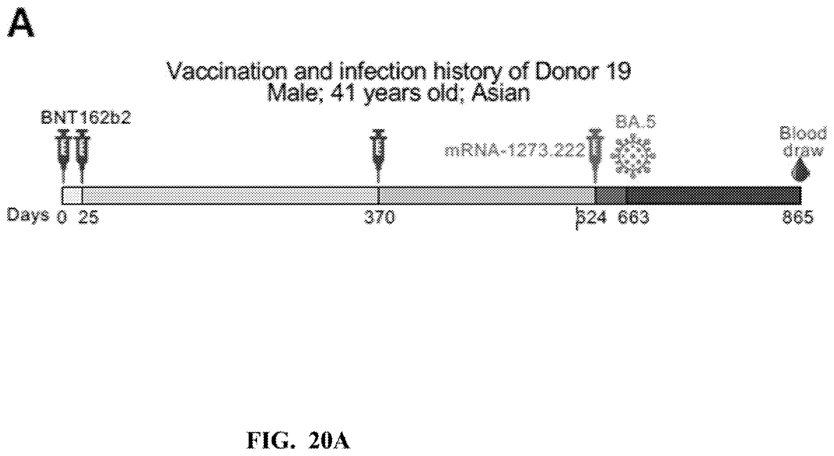

To isolate new neutralizing mAbs against SARS-CoV-2, Donor 19 who had been infected by the Omicron BA.5 subvariant despite having received three doses of the original monovalent vaccine (BNT162b2) and one dose of the WT/BA.5 bivalent vaccine (mRNA-1273.222) was studied (FIG. 20A). His serum, obtained ~7 months after breakthrough infection, strongly neutralized the D614G strain but less potently against XBB.1.5 and SARS-CoV (FIG. 1). Given the dominance of XBB.1.5 at the start of this study, XBB.1.5 and SARS-CoV spikes were used as probes to sort antigen-specific memory B cells from his peripheral blood mononuclear cells (PBMCs). The proportions of his antigen-specific B cells were 0.16% for XBB.1.5 spike, 0.067% for SARS-CoV, and 0.12% for both XBB.1.5 and SARS-CoV spikes; in contrast, PBMCs from two healthy donors showed negligible antigen-specific B cells (FIG. 1C).

Donor 19's memory B cells from quadrants 2 and 3 (FIG. 1C) were then subjected to single-cell RNA sequencing using 10× Genomics to obtain paired heavy and light chain sequences of each B cell receptor, as previously described (36). A total of 113 paired sequences were selected, and the corresponding antibodies were synthesized for in vitro characterization. A majority (75) of the mAbs bound the D614G spike at a concentration of 10 μg/ml, but only 29 neutralized either D614G or EG.5.1 in vitro (FIG. 20B). Among the neutralizing mAbs, 19-77 was chosen for further studies, because it not only bound strongly to the D614G spike and RBD by immunoassays (FIG. 2B), but also showed broadly neutralizing activity against many SARS-CoV-2 strains (FIG. 2B). Specifically, in pseudovirus assays, 19-77 neutralized D614G, Alpha, Beta, Gamma, Delta, BA.1, BA.2, BA.5, BQ.1.1, XBB.1.5, EG.5.1, BA.2.86, and JN.1 with IC₅₀ values <0.05 μg/ml. However, its neutralizing activity was lower against HK.3 and JF.1, and not detectable against JD.1.1 or SARS-CoV.

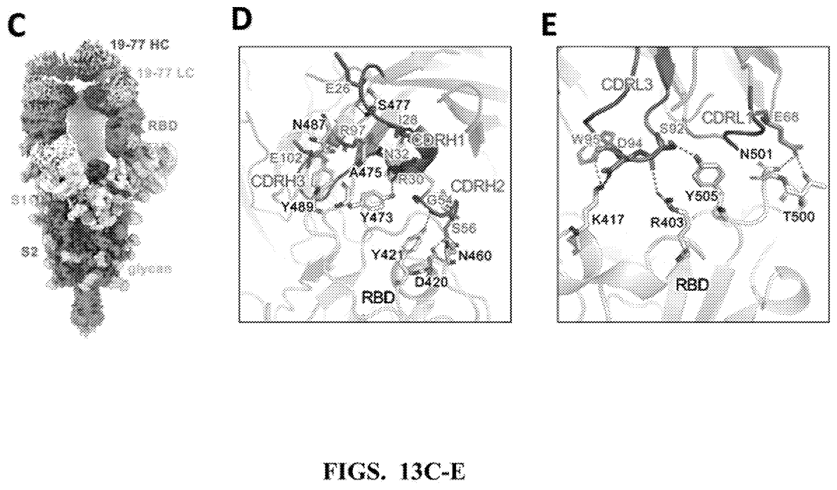

Genetic analysis revealed that the heavy chain of 19-77 utilized IGHV3-53 (FIG. 20C), showing that this antibody belonged to the well-studied VH3-53/66 class of antibodies targeting the SARS-CoV-2 spike. Its light chain originated from IGKV3-11. Notably, the CDRH3 of 19-77 contained only 9 amino acids, one of the shortest among published VH3-53/66 class mAbs (FIG. 13B). Moreover, the level of somatic hypermutation of its heavy chain was the highest observed among VH3-53/66 mAbs, and its light chain was also quite hypermutated (FIG. 13B). These striking genetic features demonstrated the uniqueness of 19-77 and prompted further studies.

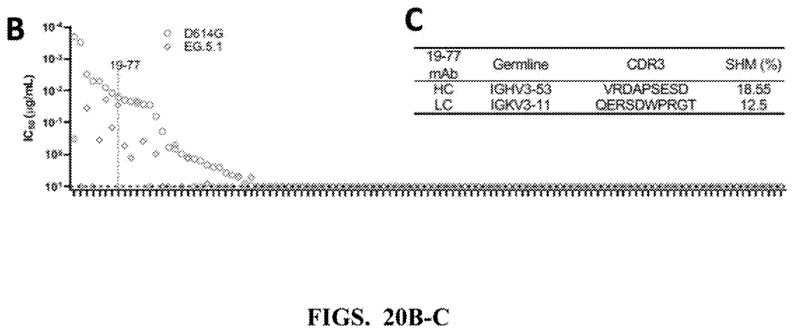

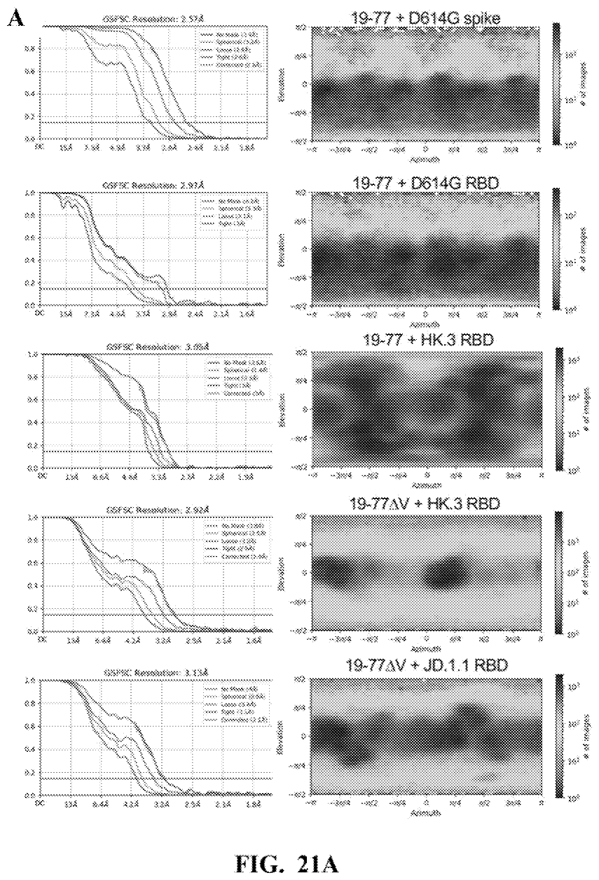

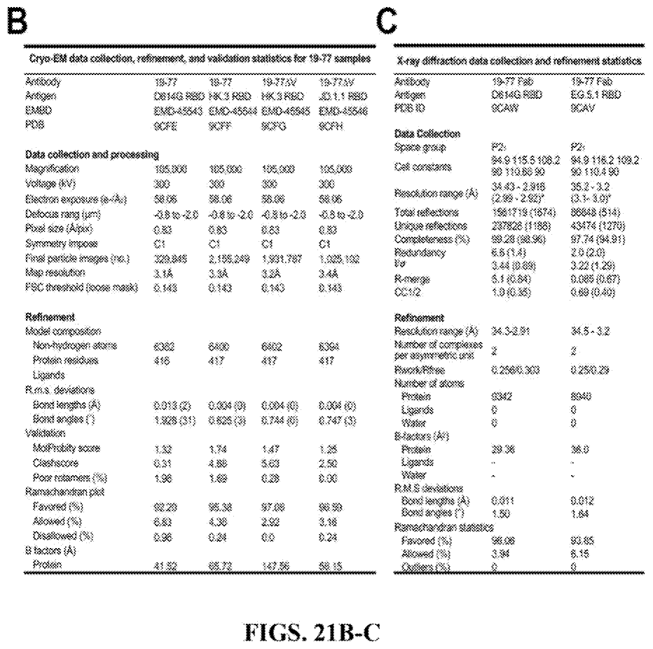

To understand the molecular basis for the neutralization breadth and potency of 19-77, its Fab fragment was then visualized in complex with the D614G spike (FIGS. 13C, 21A-B) by single-particle cryo-electron microscopy (cryo-EM). The Fab was predominantly bound to the spike protein in a three-RBD-up conformation, like other VH3-53/66 class antibodies targeting the class 1 region (37). The primary interaction between the 19-77 heavy chain and the RBD was mediated by CDRH1 and CDRH2, with hydrogen bonds to A475, N487, Y473, D420, and Y421 (FIG. 13D). The CDRH3 region formed two hydrogen bonds with the RBD: one between R97 and the backbone carbonyl of N487, and another between E102 and Y489. On the other hand, the 19-77 light chain made contacts with the apical ridge of the RBD via its CDRL1 and CDRL3 (FIG. 13E).

Structural explanation for the loss of 19-77 activity against certain Omicron subvariants

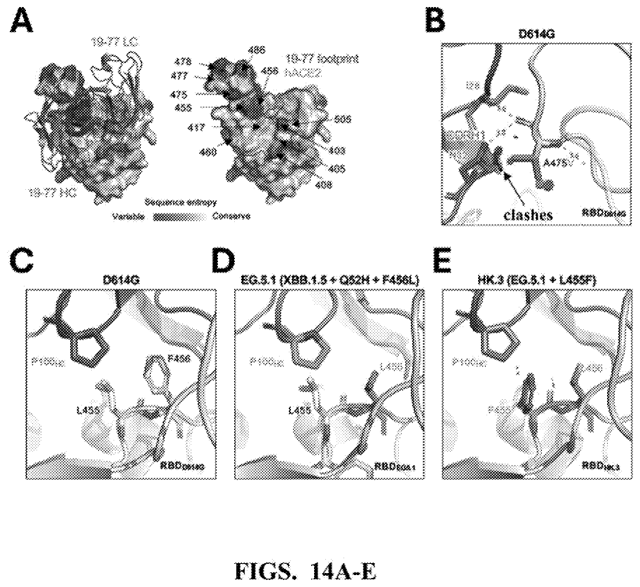

The antibody footprint of 19-77 on the RBD largely overlapped with the human ACE2 receptor binding site (FIG. 14A). Sequence conservation analysis indicated that most regions of its epitope are relatively conserved, except for the upper half of the left shoulder, which exhibits significant sequence variations at positions 455, 456, 475, 478, and 486. These specific residues have undergone frequent mutations in certain recent subvariants, such as EG.5.1, HK.3, JF.1, and JD.1.1. It is therefore not surprising that the neutralizing activity of 19-77 against HK.3 (XBB.1.5 carrying Q52H and L455F/F456L mutations) and JF.1 (XBB.1.5 carrying E180V, L455F/F456L, and K478R) decreased by 14- and 42-fold, respectively, compared with that against XBB.1.5 (FIG. 13A). The inactivity of 19-77 against JD.1.1 is likely due to its A475V mutation that causes a steric clash with CDRH1 (FIG. 14B). To further understand the resistance mechanism of L455F/F456L mutations to the neutralization by 19-77, the structure of 19-77 Fab was determined in complex with the RBD of EG.5.1 by X-ray crystallography, as well as the cryo-EM structures of the D614G RBD and the HK.3 RBD in complex with the Fabs of 19-77 and S309 (sotrovimab) (FIG. 21), a non-overlapping mAb used to increase the molecular mass for visualization by cryo-EM. Compared with the D614G RBD (FIG. 14C), the change from phenylalanine (F) to leucine (L) at residue 456 in the EG.5.1 RBD reduced hydrophobic interactions with P100HC in the CDRH3 of 19-77 (FIG. 14D). Additionally, the L455F mutation in HK.3 introduced a minor van der Waals clash with P100HC (FIG. 14E).

### Optimization of 19-77

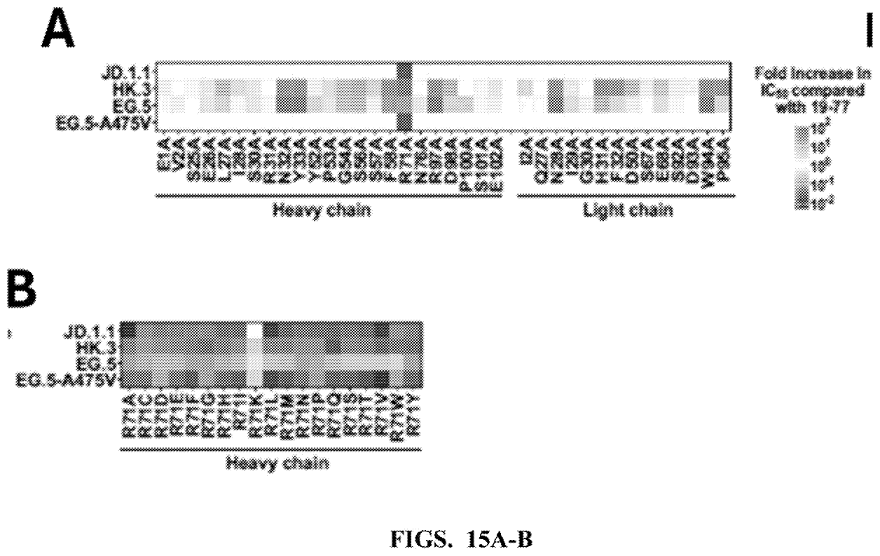

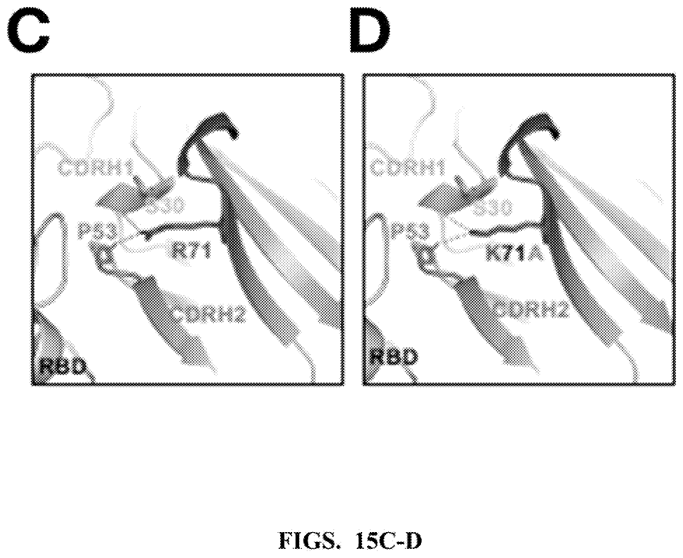

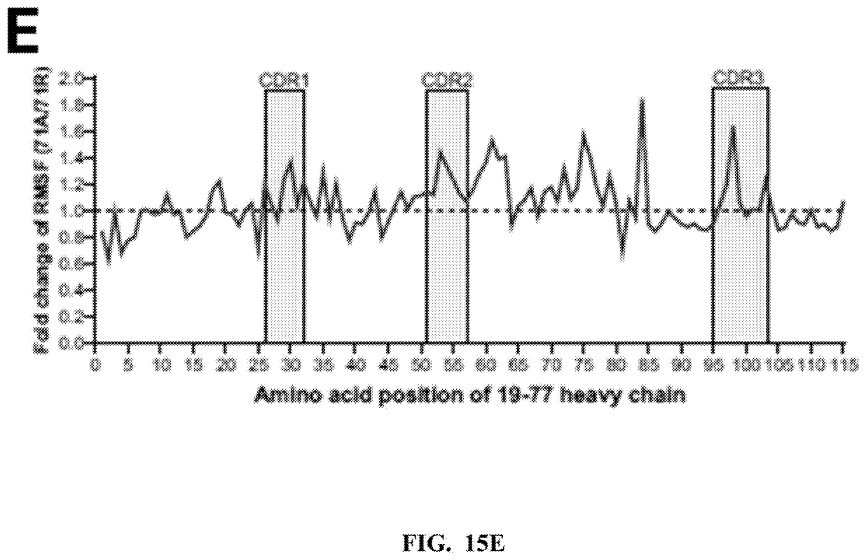

Given the minor to moderate steric clashes between the RBDs of resistant viruses and 19-77, the possibility of optimizing the antibody to accommodate for the resistance mutations was explored. Utilizing in-silico modeling and the energy-calculation algorithm FoldX38, saturation mutagenesis was performed on each residue of both the heavy and light chain, comparing the binding energy changes (ΔΔG) between each mutant and the original 19-77 in complex with the HK.3 RBD that contained the L455F/F456L mutations. Both beneficial and detrimental mutations were identified (FIG. 22A; data for light chain not shown). Interestingly, substitutions at residue R71 in framework region 3 of the heavy chain consistently predicted a moderately beneficial effect (~0.5 kcal/mol) on the binding of 19-77 to the HK.3 RBD. Alanine scanning was conducted on the antigen-contact residues of both heavy and light chains of 19-77, in addition to the residue R71, followed by testing each modified antibody for neutralization against JD.1.1, HK.3, EG.5, and EG.5-A475V (FIG. 15A, 22B-C). Only the R71A mutation significantly enhanced the neutralization activity of 19-77 against the viruses tested, including the restoration of neutralizing activity against JD.1.1 and EG.5-A475V. The other 18 possible amino-acid substitutions were introduced to the R71 position of 19-77 and then evaluated their neutralization efficacy against the same panel of resistant viruses (FIG. 22D). Except for R71K, all other substitutions resulted in not only improved neutralization activities against EG.5 and HK.3 relative to the original antibody, but also restoration of activity against JD.1.1 and EG.5-A475V. Overall, the extent of improvement for these mutant antibodies (19-77Δs) ranged from 3- to >100-fold, with aliphatic substitutions R71A, R71L, and R71V being the best (FIGS. 15B. 22D).

Structural analysis showed that R71 of 19-77 forms hydrogen bonds with S30 of CDRH1 and P53 of CDRH2, likely contributing to antibody stability (FIG. 15C). Additional modeling indicated that mutating R71 could disrupt these hydrogen bonds, with the exception of R71K, which could also form similar interactions (FIG. 15D). It was then hypothesized that R71 might restrict the motion of CDRH1 and CDRH2 of 19-77, and that mutating this arginine could lead to greater antibody flexibility. To test this hypothesis, molecular dynamics (MD) simulations were conducted for 19-77 with either arginine or alanine at position 71 of the heavy chain. The results showed that A71 increased the range of motion of the heavy-chain CDRs compared to R71. Moreover, the fluctuations in the movement of each residue for the A71 variant of 19-77 were noticeably greater than those in the original antibody, especially within the CDRs (FIG. 15E). These findings, in turn, suggested that the increased flexibility of the A71 variant could allow the antibody to better accommodate for certain mutations in the SARS-CoV-2 spike.

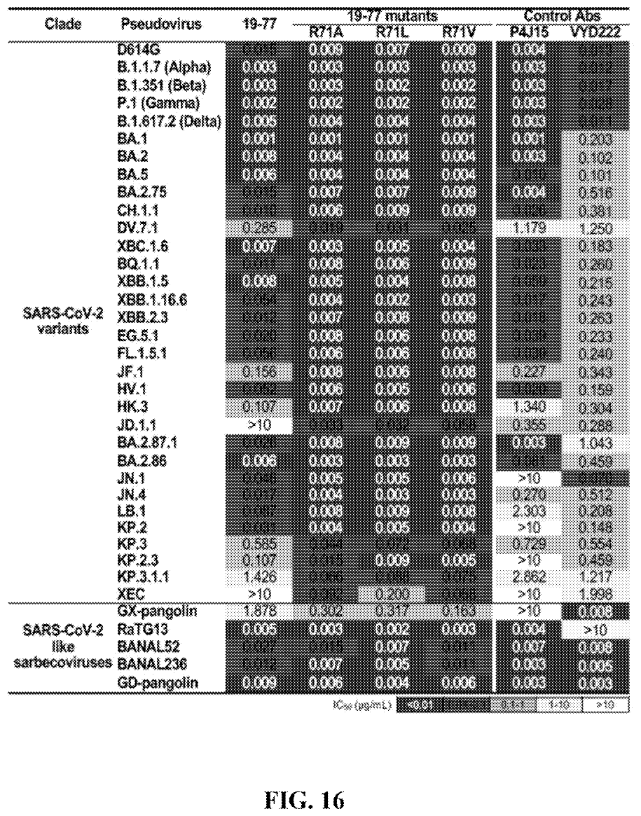

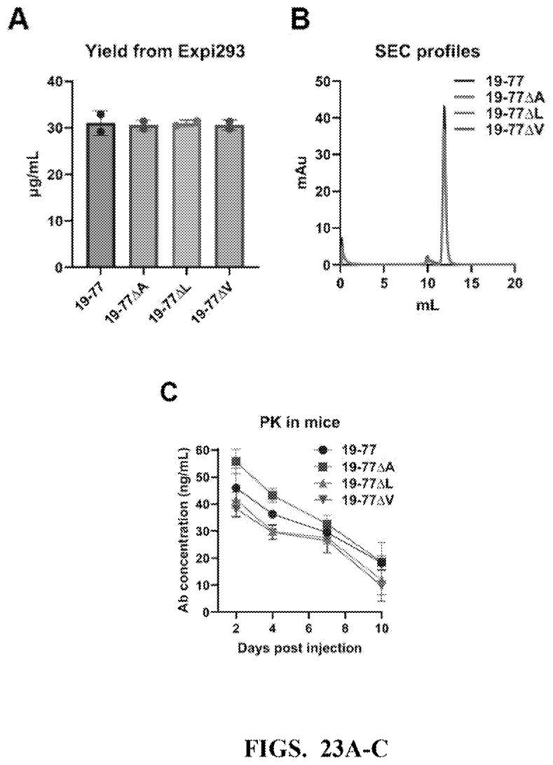

The in vitro neutralizing activity of the R71A, R71L, and R71V versions of 19-77 (designated 19-77ΔA, 19-77ΔL, and 19-77ΔV, respectively) were assessed against a large pseudovirus panel comprising of 32 SARS-CoV-2 variants and 5 SARS-CoV-2-like sarbecoviruses. Control antibodies included the original 19-77, along with the recently authorized pemivibart (VYD222)33 and a published broadly neutralizing mAb, P4J1539 (FIG. 16). The engineered 19-77 variants consistently showed better virus neutralizing potency and breadth compared to the parental antibody. Importantly, against viruses that are widely circulating today, the improvements were substantial: 7.7-to-9.2-fold for JN.1, 6.2-to-7.8-fold for KP.2, 8.1-to-13.3-fold for KP.3, 16.2-to-21.6-fold for KP.3.1.1, and >50.0-to-147.1-fold for XEC, with the latter two being most relevant presently. The activity P4J15 was either severely impaired or knocked out by JN.1 and its progeny viruses. Pemivibart lost potency against recent JN.1 sublineages, particularly KP.3.1.1 and XEC. These results showed that the optimized 19-77 mAbs possess the requisite antiviral properties to be considered as candidates for clinical development, pending a full developability assessment. Reassuringly, the three mutations at R71 did not adversely impact antibody expression, aggregation propensity, or pharmacokinetics in mice (FIG. 23).

Impact of R71 Mutations on Other VH3-53/66 Class of mAbs to the Viral Spike

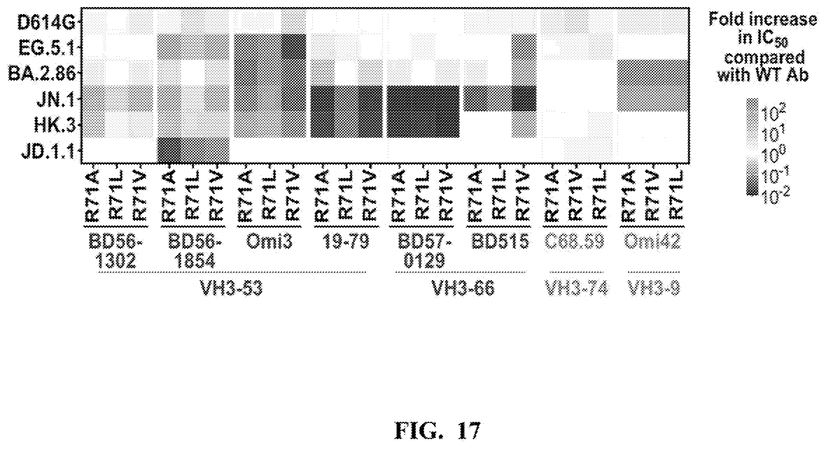

Monoclonal 19-77 belonged to the VH3-53/66, multi-donor class of SARS-CoV-2 neutralizing mAbs that target the class 1 region of RBD (37). It was tested whether the same set of R71 mutations could similarly benefit other members of this antibody class. Mutations R71A, R71L, and R71V were then introduced into five VH3-53/66 class mAbs, including BD56-130240, BD56-185440, Omi-341, 19-79 (unpublished mAb), BD57-012940, and BD-51542, as well as two non-VH3-53/66 class mAbs, C68.5929 and Omi4241, serving as controls. The original mAbs and their modified antibody variants were then assayed for their neutralizing activity against a panel of pseudoviruses (FIG. 24). All unmodified mAbs showed partial or complete loss of neutralizing activity against some later Omicron subvariants such as EG.5.1, HK.3, JD.1.1, and JN.1. In contrast, every modified VH3-53/66 mAb exhibited improved neutralization capability, albeit with varying magnitudes (3- to >100-fold), whereas no improvement was observed for non-VH3-53/66 mAbs (FIGS. 17, 24). In fact, a number of modified mAbs regained the ability to neutralize subvariants that had been completely resistant to their unmodified counterparts. For example, BD57-0129 failed to neutralize JN.1 or HK.3, but its modified counterparts neutralized both viruses robustly. The extension of the benefit of R71 mutations to mAbs other than 19-77 suggests that the responsible mechanism is not likely to operate solely at the interface with the paratope. Instead, the broader benefit observed is consistent with the hypothesis of greater conformational flexibility of the antibody CDRs discussed previously. Additionally, antibodies using VH3-53 in the human B-cell repertoire very rarely carry a mutation at R71 (FIG. 3), highlighting the uniqueness of these engineered mAbs compared to those found after SARS-CoV-2 infections.

Molecular Insights into Enhanced Virus Neutralization by 19-77ΔV

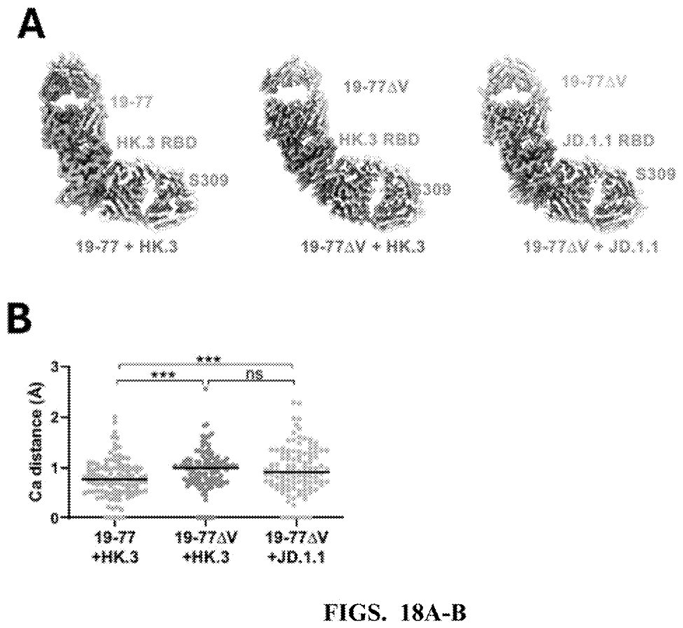

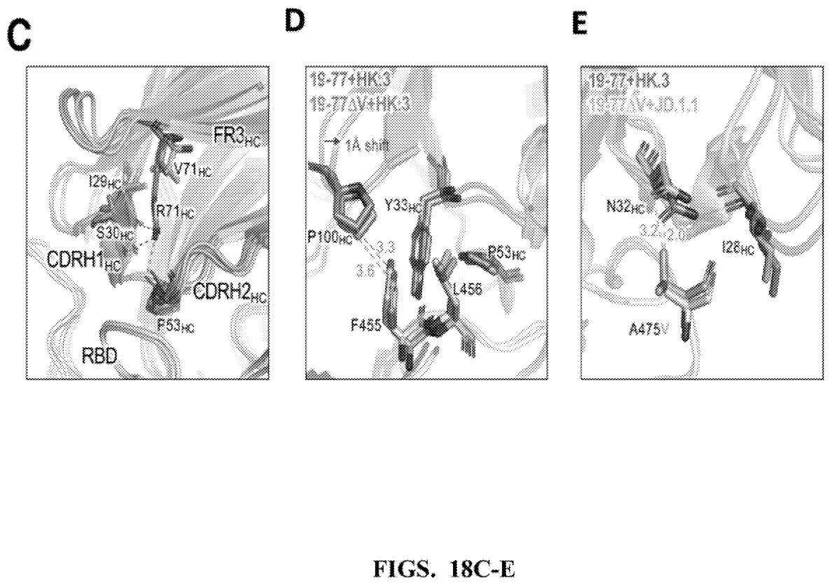

To further explore the mechanism of the enhancement of virus neutralization by mutating R71, two additional cryoEM structures were determined: the Fabs of 19-77ΔV and S309 in complex with HK.3 or JD.1.1 RBD at 2.9 Å and 3.1 Å resolution, respectively (FIGS. 18A, 21). These structures were compared by aligning their RBDs with that of the 19-77-D614G complex and measuring the alpha carbon (Cα) distances for each heavy chain residue in 19-77 against the D614G complex. The analysis showed that the Cα distances in 19-77ΔV, when bound to HK.3 and JD.1.1 RBDs, were significantly greater (0.99 Å and 0.91 Å, respectively) compared to the original 19-77 in complex with HK.3 RBD (0.76 Å) (FIG. 18B). Moreover, the motion of the CDRH1 and CDRH2 were observed directly across these structures, and the valine substitution at residue 71 resulted in the loss the hydrogen bonds that had existed between R71 and the residues S30 and P53 in the heavy chain (FIG. 18C). In addition, the CDRH3 in 19-77ΔV exhibited a shift of 1 Å compared to the original 19-77, resulting in an increased distance (from 3.3 Å to 3.6 Å) between residues P100 and F455 (FIG. 18D). This change mitigated the slight van der Waals clash between these two residues in HK.3 RBD and 19-77 complex (FIG. 14E). Lastly, the distance between V475 in the JD.1.1 RBD and N32 of the heavy chain of 19-77ΔV increased to 3.2 Å from the 2.0 Å found in the original 19-77 bound to D614G RBD (FIG. 18E), showing the mitigation of steric hindrance caused by the A475V mutation. Collectively, these findings support the notion that 19-77Δ mAbs are indeed more flexible and therefore more accommodating for the mutations found in emerging SARS-CoV-2 variants.

### In Vitro Selection of SARS-CoV-2 Resistant to 19-77ΔV

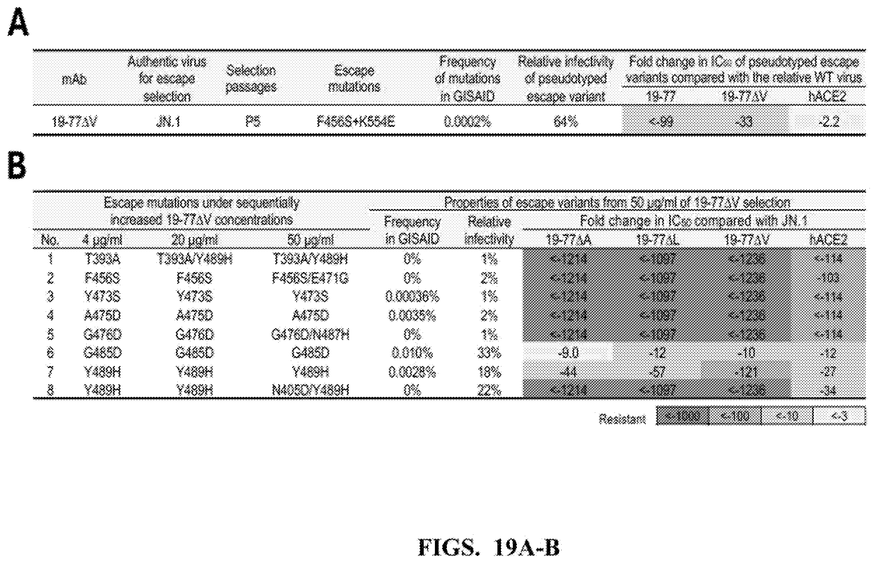

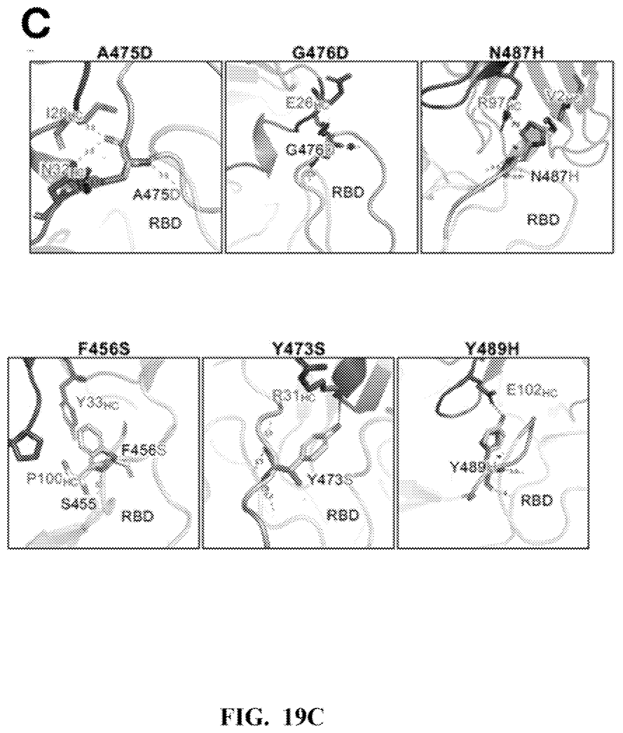

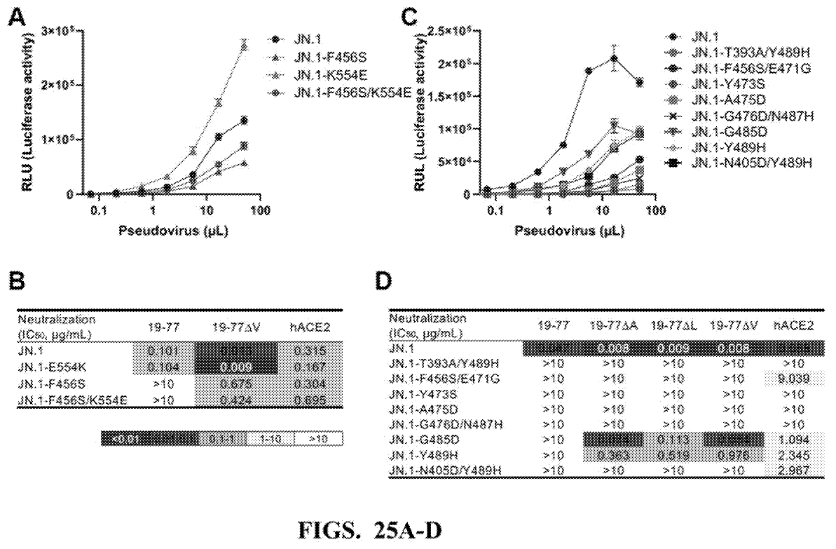

Although the SARS-CoV-2-neutralization breadth and potency of the optimized 19-77 variants were impressive (FIG. 16), the expectation was that the virus will still find a way to escape given its history of evading all COVID-19 mAbs authorized to date. Two sets of in vitro studies were performed to define its mutational pathways to resist 19-77ΔV. First, serial passaging of the authentic JN.1 virus was performed in increasing antibody concentrations (0.1 to 50 μg/mL) in Vero-ACE2-TMPRSS2 cells over 15 days (under 3 days per passage). After five passages, a resistant virus was sequenced and found to carry mutations F456S and K554E (FIG. 19A). When these mutations were introduced into the JN.1 pseudovirus, the resultant pseudovirus showed a 33-fold greater resistance to 19-77ΔV, and even more to 19-77. Additional studies showed that the antibody evasion was attributable solely to F456S, while K554E partially compensated for the loss in viral fitness (FIG. 25A-B). Notably, this combination of mutations is extremely rare (0.0002%) in the GISAID database.

Second, a replication-competent VSV bearing the JN.1 spike was utilized to select for variants that could escape from 19-77ΔV. This selection was performed in 66 replicates in 24-well plates, involving four rounds of selection over 8 days in Vero-E6-TMPRSS2-T2A-ACEs cells with increasing concentrations of 19-77ΔV (0.4 to 50 μg/mL). At the end of this process, escape variants were detected in 11 wells, and sequencing of their spike genes revealed 8 distinct single or double mutations (FIG. 19B). Notably, all these mutations were rarely found in the GISAID database, with frequencies ranging from 0% to 0.01%, and each of the pseudoviruses constructed with these mutations exhibited impaired infectivity compared to the JN.1 pseudovirus, ranging from 1% to 33% (FIGS. 19B, 25C). Furthermore, these pseudoviruses demonstrated increased resistance to soluble hACE2 inhibition (FIGS. 19B, 25D), indicating a loss in receptor affinity, perhaps accounting in part for their reduced infectivity. Single mutations G485D and Y489H, both residing in the RBD, decreased the neutralization activity of 19-77ΔA/L/V considerably (9- to 121-fold), whereas the remaining mutations completely abolished the neutralization activity of the three optimized mAbs (FIGS. 19B, 25D).

In silico structural analysis showed that each of the escape viruses contained a mutation within the epitope of 19-77ΔV that impaired antibody binding (FIG. 19C). A475D, G476D, and N487H caused steric hindrance to antibody binding, F465S mutation and residue S455 in JN.1 significantly reduced hydrophobic interactions between the antibody and RBD. Furthermore, both Y473S and Y489H abolished hydrogen bonds with the antibody, contributing to the dramatic drop in neutralization observed for the escape viruses with these mutations.

### Discussion

This study presents findings on the characterization of a human IGHV3-53-derived monoclonal antibody, 19-77, which exhibited neutralizing activity against most but not all SARS-CoV-2 variants (FIGS. 13A, 16). Strikingly, 19-77 featured the shortest CDRH3 (9 amino acids) while containing the highest degree of somatic hypermutations among its class of mAbs (FIG. 13B). Structural analysis revealed that 19-77 recognized the spike RBD in the “up” position (FIG. 13C), and its epitope overlapped with the ACE2 footprint (FIG. 14A). Subsequent in silico energy calculations (FIG. 22A) and empirical mutagenesis experiments (FIG. 15A-B) yielded modified antibodies 19-77ΔA, 19-77ΔL, and 19-77ΔV, each of which neutralized all 32 SARS-CoV-2 variants tested, including KP.3.1.1 and XEC that are so prevalent today (FIG. 16). These optimized antibodies joined a limited list of human mAbs that possess sufficiently potent neutralizing activity against all viral variants, such as SA5543, BD55-120544, and VIR-722945 that target an epitope overlapping with that of 19-77, as well as CYFN1006-146 that targets a RBD class 3 epitope. However, this study showed that SARS-CoV-2 could easily escape from neutralization by 19-77ΔV in vitro (FIG. 19). In fact, the virus found multiple solutions to evade this optimized antibody, albeit with a fitness cost. Such an outcome was, more or less, expected given how SARS-CoV-2 has evolved in the population to render inactive all clinical mAbs authorized prior to 2024. Indeed, pemivibart (VYD222) that was authorized only months ago for use as prophylaxis against COVID-19 in immune deficient subjects is already seriously threatened by the emergence of KP.3.1.1 and XEC subvariants (35, 47, 48).

It has been a daunting challenge, as well as a frustrating endeavor, to develop therapeutic or prophylactic mAbs to keep up with the rapid pace of SARS-CoV-2 evolution. Yet the reality is that millions of individuals worldwide are sufficiently immunocompromised that they cannot benefit from the protection conferred by COVID-19 vaccines. Such persons need effective prophylaxis with passively administered virus-neutralizing mAbs, as was conferred previously by the combination of tixagevimab and cilgavimab known as Evusheld (49) and presently by pemivibart (33). Going forward, it is hypothesized one or all of the optimized 19-77Δ mAbs would qualify as a compelling candidate for clinical development given its superior potency and breadth against all SARS-CoV-2 variants known to date (FIG. 16), as well as its lack of discernible developability challenges to date (FIG. 23). The potential clinical utility of an optimized 19-77Δ could be further improved if used in combination with a non-competing, broadly neutralizing mAb like CYFN1006-146.

Described herein is a unique strategy for optimizing the activity of a monoclonal antibody by increasing its conformational flexibility. Traditionally, antibody engineering had been focused primarily on increasing the binding affinity using a number of approaches, including chain shuffling (50, 51), site-directed mutagenesis (52), phage and other display technologies (53-57), and structure-based or artificial-intelligence-guided methods (58-60). Such methodologies, typically, had been designed to increase antibody affinity by modifying the CDR residues at the antibody-antigen interface. Other antibody approaches to optimize antibody activity included the use of multivalency with the creation of “multabodies” (61), or the strategic addition of a glycan to bulk up a steric hindrance effect (62). The framework regions of a mAb were seldom touched, because they are crucial for maintaining the proper orientation and conformation of the CDRs (63, 64). In this study on 19-77, however, in silico energy calculations indicated that a single amino-acid substitution at residue 71 in framework region 3 of the heavy chain could improve antibody binding to the viral spike (FIG. 22A), which was proven correct experimentally by replacing the arginine with any other natural amino acid except for lysine (FIGS. 15A-B, 16). The improved activity of optimized 19-77Δ mAbs was attributable to the loss of hydrogen bonding by R71 (FIG. 15C and FIG. 18C), leading to greater flexibility of the CDRs as shown by molecular dynamics (FIG. 15E) and structural analyses (FIG. 18). This enhanced conformation flexibility, in turn, allowed each optimized antibody to be more tolerant of mutations within or near its epitope, ultimately resulting in the striking SARS-CoV-2 neutralization potency and breadth observed (FIG. 16). Remarkably, this optimization strategy was also successfully applied to 6 other IGHV3-53/3-66-derived human mAbs that target the same epitope cluster as 19-77 (FIG. 17). But its applicability did not extend to other SARS-CoV-2-neutralizing mAbs that utilize other VH germline genes, suggesting a specific mutation at framework residue 71 is not likely to confer a generalizable benefit to other mAbs. Nevertheless, the general concept of modifying antibody conformational flexibility should be further explored in other settings by other means. Increasing conformational rigidity could lead to improved antibody-antigen affinity, as has been reported for an IGHV4-59-encoded anti-lysozyme mAb (65). On the other hand, increasing conformational flexibility, as shown in this study, could allow the antibody to better tolerate sequence variations in the target antigen. Such an approach may be useful for monoclonal antibodies directed to polymorphic antigens or surface proteins of rapidly evolving viruses such as HIV-1, coronaviruses, influenza viruses, and hepatitis C virus. It may be another important conceptual tool in the antibody engineering armamentarium.

### Methods

### Human Subject

Blood sample from Donor 19, a 41-year-old Asian male, was collected at Columbia University Irving Medical Center. Donor 19 was confirmed for a BA.5 infection by PCR sequencing (Single Nucleotide Polymorphisms) and provided written informed consent. Sample collections were performed under protocols reviewed and approved by the Institutional Review Board of Columbia University. Clinical information of Donor 19 is provided in Extended FIG. 1A.

### Cell Lines

Vero-E6 (CRL-1586) and HEK293T (CRL-3216) cells were purchased from the American Type Culture Collection (ATCC). Expi293 cells (A14527) were purchased from Thermo Fisher Scientific. Vero-E6-TMPRSS2-T2A-ACE2 (NR-54970) were obtained from BEI Resources. 293T-ACE2 were kindly provided by Dr. Jesse D. Bloom. Morphology of each cell line was confirmed visually before use. All cell lines tested mycoplasma negative. Vero-E6 and Vero-E6-TMPRSS2-T2A-ACE2 cell lines are from African green monkey kidneys. HEK293T, 293T-ACE2, and Expi293 cells are of female origin.

### Plasmid Construction

SARS-CoV-2 spike-expressing plasmids for D614G, Alpha, Beta, Gamma, Delta and Omicron were previously generated13, 20, 21, 27. Expressing constructs for the spike proteins of SARS-CoV-2-like sarbecoviruses were either previously generated (66) or newly generated by synthesizing (GenScript) spike genes and then cloned into the pCMV3 vector. To generate the expression constructs for soluble spike trimer (S2P) proteins, the ectodomains (1-1208aa, numbering based on WA1) of the spikes were PCR amplified and cloned into the paH vector and then introduced K986P and V987P substitutions, as well as a “GSAS” substitution of the furin cleavage site (682-685aa in WA1) into the spikes (67). SARS-CoV S2P was fused with an AVI tag at the C terminus and D614G and XBB.1.5 S2P spikes were tagged with a 6×His tail also at the C terminus. To make SARS-CoV-2 RBD-expressing construct, RBD region (319-537aa) of each variant was fused with a 6×His tag and then cloned into the p3BNC vector. All constructs were confirmed by Sanger sequencing.

Antibody expressing constructs were generated as previously described (36). The variable regions of heavy and light chains for each antibody were synthesized (GenScript) and then cloned into the gWiz vector. To make spike/antibody plasmid constructs carrying individual mutations, the Q5® Site-Directed Mutagenesis Kit (NEB) was utilized following the manufacturer's instructions.

### Protein Purification

To make human ACE2-Fc (hACE2) protein, pcDNA3-sACE2-WT(732)-IgG1 (68) (Addgene plasmid #154104, gift of Erik Procko) plasmid was transfected into Expi293 cells using 1 mg/mL polyethyleneimine (PEI) at a ratio of 1:3, and the supernatants were collected after five days. hACE2 was purified from the cell supernatant by using rProtein A Sepharose (GE). For antibody purification, both heavy and light chains of each antibody were transfected at a ratio of 1:1 into Expi293 cells using PEI. And the expressed antibody in the cell supernatant was purified using the same method as for hACE2 purification. For the spike trimer proteins or RBD proteins, paH-spike or p3BNC-RBD, respectively, was transfected into Expi293 cells using PEI at a ratio of 1:3, and the supernatants were collected five days later. The His tagged and the AVI tagged proteins were purified using Excel resin (Cytiva) and Agarose bound Galanthus nivalis lectin (VectorLabs, AL-1243-5) according to the manufacturers' instructions. The molecular weight and purity were checked by running the proteins on SDS-PAGE.

After purification of 19-77 and 19-77ΔA/L/V, 100 μg of each antibody was prepared and run through a Superdex 200 Increase 10/300 GL column to generate their size exclusion chromatography (SEC) profiles. AVI tagged SARS-CoV S2P protein were biotinylated using the BirA biotin-protein ligase standard reaction kit (Avidity LLC; BirA500) following the manufacturer's instructions.

### ELISA

50 ng per well of an antigen such as S2P spike, NTD (ACROBiosystems, S1D-C52H6), RBD (ACROBiosystems, SPD-C52H1), RBD-SD1 (Exonbio, 19Cov-S130), S1 (ACROBiosystems, S1N-C52H3), or S2 (ACROBiosystems, S2N-C52H2) was coated onto ELISA plates at 4 °C overnight. The ELISA plates were then blocked with 300 μL of blocking buffer consisting of phosphate-buffered saline (PBS) with 1% bovine serum albumin and 20% bovine calf serum (Sigma-Aldrich) at 37 °C for 2 hours. Afterwards, 100 μL of 5-fold serially diluted antibodies was added and then incubated at 37 °C for 1 hour. Next, 100 μL of 10,000-fold diluted Peroxidase AffiniPure goat anti-human IgG Fcγ fragment-specific antibody (Jackson ImmunoResearch, catalog no. 109-035-170, RRID: AB_2810887) was added into each well and incubated for another 1 hour at 37 °C. The plates were washed between each step with PBST (0.5% Tween-20 in PBS). Last, 3,3′,5,5′-tetramethylbenzidine (TMB) substrate (Sigma-Aldrich) was added and incubated before the reaction was stopped using 1 M sulfuric acid. Absorbance was measured at 450 nm.

### Antibody Pharmacokinetics in Mice

100 μg (1 mg/mL) of each antibody was intraperitoneally (ip) injected into Balb C mice (3 mice for each antibody). Mouse blood was collected on day 2, 4, 7 and 10 post ip injection and antibody concentrations in serum were measured by ELISA. Briefly, 100 ng goat anti-human IgG Fc antibody (Cat: 109-005-008, JacksonImmunoResearch) was coated per well in 96-well plates overnight and the ELISA plates were then blocked with 300 μL of blocking buffer consisting of PBS with 1% bovine serum albumin and 20% bovine calf serum (Sigma-Aldrich) at 37 °C for 2 hours. Afterwards, 100 μL of 3-fold serially diluted serum or 2-fold serially diluted purified antibody was added and then incubated at 37 °C for 1 hour. Next, 100 μL of 10,000-fold diluted Peroxidase AffiniPure goat anti-human IgG H+L HRP (Cat: 109-035-088, JacksonImmunoResearch) was added into each well and incubated for another 1 hour at 37 °C. The plates were washed between each step with PBST (0.5% Tween-20 in PBS). Last, TMB substrate (Sigma-Aldrich) was added and incubated before the reaction was stopped using 1 M sulfuric acid. Absorbance was measured at 450 nm.

### Pseudovirus Production and Infectivity

SARS-CoV-2 pseudoviruses were generated as previously described (36). In brief, HEK293T cells were transfected with a spike-expressing construct using 1 mg/mL PEI and then infected with VSV-G pseudotyped ΔG-luciferase (G*ΔG-luciferase, Kerafast) one day post-transfection. Two hours after infection, cells were washed three times with PBS, changed to fresh medium, and then cultured for one more day before the cell supernatants were harvested. Pseudoviruses in the cell supernatants were clarified by centrifugation, aliquoted, and stored at −80 °C.

To evaluate the infectivity of the pseudotyped escape variants of 19-77 and 19-77ΔV, fresh pseudoviruses without freezing and thawing were serially titrated from 50 μL with a dilution factor of 3 and then inoculated into Vero-E6 cells. After overnight culture, Vero-E6 cells were harvested and quantified for luciferase activity using the Luciferase Assay System (Promega).

### Pseudovirus Neutralization Assay

To normalize the viral input between assays before conducting the neutralization assays, pseudoviruses of SARS-CoV and SARS-CoV-2 variants and SARS-CoV-2-like sarbecoviruses were titrated on Vero-E6 cells and 293T-ACE2 cells. Heat-inactivated serum from Donor 19 was serially diluted starting from 1:25 with a dilution factor of four. Monoclonal antibodies were 5-fold serially diluted starting from 20 μg/mL in 96 well plates in triplicate. Then, 50 μL of diluted pseudovirus was added and incubated with 50 μL serial dilutions of serum or mAb for 1 hour at 37 °C. During the co-culture, target cells were trypsinized, resuspended with fresh medium, and then added into virus-sample mixture at a density of 4-10×104 cells/well. The plates were incubated at 37 °C for ~12 hours before luciferase activity was quantified using the Luciferase Assay System (Promega) using SoftMax Pro v.7.0.2 (Molecular Devices). Neutralization ID50 values for sera and IC₅₀ values for antibodies were calculated by fitting a nonlinear five-parameter dose-response curve to the data in GraphPad Prism v.10.

### Selection of Escape Mutations

SARS-Cov-2 isolate Omicron JN.1 (BEI NR-59693) was mixed with serial five-fold dilutions of 19-77ΔV antibody at MOI of 0.2 and incubated for 1 h. Following incubation, the mix was overlaid on a 24-well plate bearing a monolayer of Vero-ACE2-TMPRSS2 cells (BEI NR-54970) to a final volume of 1 mL. Plates were incubated at 37 °C/5% CO2 for 70 h till cytopathic effect (CPE) was complete (100%) in virus control wells bearing no antibody. At this time, all wells with antibody dilutions were scored to determine the 50% inhibition titer (EC50) and supernatant collected from this well was used for subsequent round of selection. Passaging of the progeny over new Vero-ACE2-TMPRSS2 cells continued till each of the virus variant was able to form CPE in the presence of 50 μg/mL of the antibody. The resulting supernatant was then collected, and RNA was extracted using QiaAMP Viral RNA kit (Qiagen 57704). cDNA was obtained using Superscript IV enzyme (Thermo Scientific 18090010). Spike gene from the cDNA was amplified using limiting dilution nested PCR and sequenced using Sanger sequencing (Genewiz). Multiple clones from limiting dilution nested PCR were sequenced to confirm the dominant mutants in the pool of the resulting progeny viruses and a percentage of their prevalence was calculated from the total number sequenced. At least 8 clones were sequenced from each of the passages reported in FIG. 7.

To generate recombinant replication-competent VSV-ΔG bearing the JN.1 spike protein (VSV-ΔG-JN.1), the pVSVΔG-SARS-CoV-2-S_nLucP plasmid was purchased from Kerafast (Cat #EGA292) and its encoding SARS-CoV-2-S gene was replaced with JN.1 spike gene to create pVSVΔG-JN.1_nLucP. The pVSVΔG-JN.1_nLucP plasmid and a set of helper plasmids, including VSV-N, VSV-P, VSV-L, and VSV-G (Kerafast, Cat #EH1012), were then transfected into 293T cells at a ratio of 5:3:5:1:8 using 1 mg/mL PEI-MAX. Before transfection, 293T cells were rinsed with serum-free DMEM, incubated with Vaccinia vTF7-3 (Imanis Life Sciences, Cat #REA006) at a MOI of 5 for 45 minutes, and then replaced with fresh medium. Two days post-transfection, the supernatant was harvested and filtered through a 0.22 μm filter to remove cell debris and Vaccinia vTF7-3. The rVSV-ΔG-JN.1 generated from 293T cells was then serially diluted with a dilution factor of 5 and inoculated into Vero-E6-TMPRSS2-T2A-ACE2 cells in 24-well plates for 1 hour. The virus was then washed away, and the cells were cultured at 37 °C/5% CO2 for 16-24 hours. Vero-E6-TMPRSS2-T2A-ACE2 cells were monitored, and the virus was harvested from wells in which only one plaque was observed, then filtered and stored at −80 °C. The rVSV-ΔG-JN.1 generated from Vero-E6-TMPRSS2-T2A-ACE2 cells was titrated on Vero-E6-TMPRSS2-T2A-ACE2 cells before use. To select escape viruses, rVSVΔG-JN.1 was incubated with 0.4 μg/mL of 19-77ΔV for 1 hour before being added to Vero-E6-TMPRSS2-T2A-ACE2 cells in 24-well plates at a MOI of 0.01. A total of 66 replicates were set up. Two days after coculture, cell supernatants from wells with CPE were harvested, and 100 μL of each supernatant was incubated with 4 μg/mL of 19-77ΔV for 1 hour before another round of infection in pre-seeded Vero-E6-TMPRSS2-T2A-ACE2 cells in 24-well plates. Two days later, supernatants containing escape viruses were further harvested and selected the same way using 20 μg/mL, and then 50 μg/mL, of 19-77ΔV in a stepwise manner. mRNAs of the escape viruses in the supernatants were then extracted using the viral RNA/DNA purification kit (MACHEREY-NAGEL, Cat #740643) and reverse-transcribed to cDNA using the SuperScript™ VILO™ cDNA Synthesis Kit (Invitrogen, Cat #11754). The RBD genes of the escape viruses were then amplified using primers 5′-GGGCATCTACCAGACCAGCAACTTCA-3′ and 5′-GAACACATTGCTGCCTGTGCTGT-3′ and sequenced.

### Antigen-Specific Memory B Cell Sorting and Single-Cell B Cell Receptor Sequencing

Peripheral blood mononuclear cells from Donor 19, and two healthy donors were stained with the LIVE/DEAD Fixable Yellow Dead Cell Stain Kit (Invitrogen) at ambient temperature for 20 min, followed by washing with RPMI 1640 complete medium [RPMI 1640+10% fetal bovine serum (FBS)+penicillin/streptomycin (P/S) (100 U/mL)] and incubation with 10 g/mL XBB.1.5 S2P protein and biotinylated SARS-CoV S2P at 4 °C for 45 min. Afterwards, the cells were washed again and incubated with a cocktail of flow cytometry and Hashtag antibodies, consisting of CD3 PerCP-Cy5.5, CD19 APC/Cyanine 7, CD27 APC, IgM FITC, anti-His PE/Dazzle™ 594, Streptavidin BV421, and human Hashtag 3 at 4 °C for 1 hour. Stained cells were then washed, resuspended in RPMI 1640 complete medium, and sorted for SARS-CoV and/or XBB.1.5 S2P trimer-specific memory B cells (CD3-CD19+CD27+IgM-antigen+live single lymphocytes) by flow cytometry. The sorted cells were mixed with spike-in CD3+ cells and loaded into a 10× Chromium chip of the 5′ Single Cell Immune Profiling Assay (10× Genomics) at the Columbia University Single-Cell Analysis Core. Library preparation and quality control were performed according to the manufacturer's protocol and sequenced on a NextSeq 500 sequencer (Illumina).

### Identification of Spike-Specific Antibody Transcripts

Antibody transcripts specific to the XBB.1.5 S2P spike and SARS-CoV spike trimers were identified following previously established methods (36). The assembly of full-length antibody transcripts was performed utilizing the Cell Ranger V(D)J analysis software (version 3.1.0, 10× Genomics), employing default settings with the GRCh38 V(D)J germline sequence version 2.0.0 as the reference genome. To differentiate between cells captured in the antigen-specific sorting process and those added as spike-ins, the count module of Cell Ranger was used to quantify the presence of all hashtag oligonucleotides in each cell based on Next Generation Sequencing (NGS) raw data. Identification of high-confidence antigen-specific cells was achieved using the following criteria: 1) A minimum of 100 copies of the antigen-specific hashtag was required for a cell to be classified as antigen-specific, 2) Given that hashtags might detach from their original cells and attach to others within the sample, a cell was considered truly antigen-specific only if the copy number of its specific hashtag was at least 1.5 times higher than that of any non-specific hashtag present, 3) Cells deemed to be of low quality were excluded based on the cell quality assessment algorithm used by Cell Ranger, 4) Only cells expressing both productive heavy and light chain antibody gene pairs were retained, 5) In instances where a cell exhibited more than two transcripts for heavy and/or light chains, transcripts supported by fewer than three unique molecular identifiers (UMIs) were discarded, and 6) Cells sharing identical heavy and light chain sequences, potentially indicative of mRNA contamination, were consolidated into a single cell entry.

### Antibody Transcript Annotation

Transcripts specific to the antigen were analyzed and annotated with SONAR version 2.0, following previously established procedures. Assignment of V(D)J gene segments to each transcript was conducted via BLASTn, employing specialized parameters against a germline gene repository sourced from the International ImMunoGeneTics (IMGT) information system database. The identification of the Complementarity Determining Region 3 (CDR3) utilized BLAST alignments of the V and J segments, focusing on the conserved second cysteine within the V segment and the WGXG (for heavy chains) or FGXG (for light chains) motifs in the J segment, with “X” indicating any amino acid. Isotype determination for heavy chain transcripts was achieved by analyzing Constant domain 1 (CH1) sequences against a human CH1 gene database from IMGT, using BLASTn with standard parameters. The CH1 allele presenting the lowest E-value was selected for precise isotype classification, adhering to a BLAST E-value cutoff of 10e-6. Transcripts with incomplete V(D)J segments, frameshifts, or extraneous sequences beyond the V(D)J region were discarded. The filtered transcripts were then aligned to their corresponding germline V gene using CLUSTALO, and levels of somatic hypermutation were quantified through the Sievers method. In instances where cells possessed multiple high-quality heavy or light chains, potentially indicative of doublets, combinations of all H and L chains were generated.

### Crystallization and Data Processing

19-77 Fab was produced by digestion of IgG with immobilized Endoproteinase Lys-C(Sigma Aldrich) equilibrated with 25 mM Tris pH 8.5 and 1 mM EDTA for 3 h. The resulting Fab was further purified from the cleaved Fc domain by cation exchange chromatography. Fab purity was analyzed by SDS-PAGE and buffer-exchanged into 20 mM Tris, 150 mM, pH 7.4 prior to cryo-EM/Crystallization experiments.

19-77/SARS-CoV-2-RBD and 19-77/EG5.1-CoV-2-RBD complexes were prepared by mixing each of the protein components at an equimolar concentration and incubating overnight at 4 °C. Protein complexes were then isolated by gel filtration on a Superdex-200 column (Cytiva, GE Healthcare). Fractions containing complexes were pooled and concentrated to 12.0 mg/mL in SEC buffer. Screening for initial crystallization conditions was carried out in 96-well sitting drop plates using the vapor-diffusion method with a Mosquito crystallization robot (TTP LabTech) using various commercially available crystallization screens: MSCG-1 (Anthracene), Proplex and LMB (Molecular dimensions). Diffraction quality crystals were obtained after seven days in the following condition for 19-77/SARS-CoV-2-RBD: 0.1 M NaCl, 0.1 M Tris pH 7.5, 12% w/v PEG 4000, and the following condition for 19-77/EG5.1-CoV-2-RBD: 75% MPD and 0.1 M HEPES pH 7.5.

Prior to data collection, crystals were cryoprotected with 40% ethylene glycol supplemented in mother liquor and flash frozen in liquid nitrogen. X-ray diffraction data extending to 2.8 Å (19-77/SARS-CoV-2-RBD) and 3.2 Å (19-77/EG5.1-CoV-2-RBD) resolution were collected at 100 K on beam line 17-ID-1 (AMX) at Brookhaven National Laboratory. Diffraction data were processed with XDS (69) and scaled using AIMLESS (70) from the CCP4 software suite (Collaborative Computational Project Number 4, 1994) (71). Molecular replacement was performed with PHASER (72), using a previously reported RBD structure (PDB 7L5B) and for 19-77 Fab, heavy chain (PDB 7XIK), light chain (3FIK) used as search models. Manual rebuilding of the structure using COOT (73) was alternated with refinement using Phenix refine (74). The Molprobity server was used for structure validation (75) and PyMOL (version 2.1, Schrödinger, LLC) for structure visualization. A summary of the X-ray data collection and refinement statistics are shown in FIG. 21C.

### Cryo-EM Sample Preparation

Fab fragments of antibodies were produced by digestion of IgG with immobilized Endoproteinase Lys-C(Sigma-Aldrich) equilibrated with 25 mM Tris, pH 8.5, and 1 mM EDTA for 3 h. The resulting Fabs were purified by ion-exchange chromatography on a mono-Q column.

For the structure of 19-77 bound to D614G spike, the complex was made by mixing purified SARS-CoV-2 S2P D614G spike protein with Fab in a 1:3 molar ratio (spike protomer:Fab) in PBS, pH 7.4, such that the final concentration of spike was 1 mg/mL. This mixture was incubated on ice for 1 h.

For the structures of the ternary complex of 19-77, RBDs, and S309, complexes were made by mixing purified SARS-CoV-2 RBD with Fabs in a 1:1.2 molar ratio in PBS, pH 7.4, and further purified using size exclusion chromatography (SEC) using a Superdex 200 Increase column. The resulting complex peak was then concentrated to 4 mg/mL and held on ice until vitrification.

Before freezing, 0.005% (w/v) n-Dodecyl β-D-maltoside (DDM) was added to deter preferred orientation and aggregation during vitrification. Cryo-EM grids were prepared by applying 3 μL of sample to a freshly glow-discharged carbon-coated copper grid (CF 1.2/1.3 300 mesh); the sample was then vitrified in liquid ethane using a Vitrobot Mark IV with a wait time of 30 s, a blot time of 3 s, and a blot force of 0.

### Cryo-EM Data Collection and Analysis

Cryo-EM data for single particle analysis were collected at the Columbia Cryo-EM Facility on a Titan Krios electron microscope operating at 300 kV, equipped with a Gatan K3-BioQuantum detector and energy filter, using the Leginon7 software package. Exposures were taken at a magnification of 105,000× (pixel size of 0.83 Å), using a total electron flux of 58 e-/Å2 fractionated over 50 frames with an exposure time of 2.5 s. A random defocus range of −0.8 to −2.0 μm was used.

Data processing was performed using cryoSPARC v3.3.1.8 Raw movies were aligned and dose-weighted using patch motion correction, and the micrograph contrast transfer function (CTF) parameters were estimated using patch CTF estimation. Micrographs were picked using a blob or template picker and an initial particle set was selected using 2D classification. Further heterogenous refinement was used to 3D-classify particles and remove debris. The resulting curated particle sets were corrected for local motion and refined using homogenous refinement. Local refinement was performed for the spike dataset using a mask enveloping the RBD+Fab variable region. The default cryoSPARC auto-sharpened maps were then used to build the models. Cryo-EM data collection and consensus refinements are summarized in Extended Data FIG. 2.

### Model Building and Refinement

Initial molecular models for Fabs were generated using Alphafold Multimer (76) using paired heavy and light sequences. An RBD from PDB 7KNI, an RBD-up spike structure, was used as a starting model. For the initial 19-77 structure, the 14-7 structure (PDB 8F89), RBD (PDB 8IOV), and S309 Fab (PDB 7XSW) were used. The initial models were rigid body docked into the density map using UCSF Chimera's “fit to map” tool and combined. The Fab CDR loops were manually fitted to the density map using Coot real space refinement. The models were fit to density using the ISOLDE package in ChimeraX (77). Ramachandran outliers were corrected using ISOLDE's “flip peptide bond” feature. Real space refinement in Phenix (78) was performed to remove geometry outliers. The remaining manual adjustments were performed in Coot. Models were validated using MolProbity16 in Phenix and the PDB validation server and deposited to the PDB with accession codes: 19-77+SARS-CoV-2 D614G RBD (PDB 9CFE), 19-77+HK.3 RBD (PDB 9CFF), 19-77ΔV+HK.3 RBD (PDB 9CFG), and 19-77ΔV+JD.1.1 RBD (PDB 9CFH). A summary of data collection, processing, and model refinement statistics is shown in FIG. 21.

### In Silico Antibody Engineering

The energy changes for the binding of both the mutant and original 19-77 to the HK.3 RBD were calculated using FoldX software (38). Initially, the 19-77 and HK.3 complex was repaired and optimized using the ‘Optimize’ function. Subsequently, every position in the heavy and light chains was subjected to saturation mutagenesis to all other amino acids using the ‘BuildModel’ function. This process generated both an unmutated model and a mutant model for each mutation. The binding energies were estimated with the ‘AnalyseComplex’ function, and the changes in Gibbs free energy (ΔΔG) were calculated based on the energy difference between each mutant and its corresponding original antibody-RBD complex.

### Molecular Dynamics Analysis

Antibody 19-77, along with other VH3-53/66 class antibodies, was meticulously aligned through the RBD within each complex to ensure structural consistency. The R71A mutant antibodies were precisely engineered using the ‘Mutagenesis’ function in PyMOL version 2.5.4, provided by Schrödinger, LLC. Subsequently, the Fab regions of both the original and mutant antibodies underwent molecular dynamics simulations employing GROMACS on the WebGro server. The preprocessing was executed using the GROMOS96 54a7 force field and incorporated an SPC water model within a cubic simulation box, supplemented with 0.15M NaCl to mimic physiological ionic strength. The initial energy minimization step was set to 5000 iterations to stabilize the system. This was followed by equilibration and simulation phases under NVT/NPT conditions, conducted at a physiological temperature of 310K for 10 ns, with all other parameters set to default. To ensure the reliability of the simulation, the root-mean-square deviation (RMSD) of each system was manually monitored to verify that each simulation reached equilibrium. Additionally, the per-residue root-mean-square fluctuation (RMSF) analysis was conducted to assess the flexibility of residues within each Fab region.

### Structure Comparison Analysis

The paratope and epitope residues for 19-77 was identified using PISA with the default parameters. The gene-specific substitution profiles (GSSP) for 19-77 germline genes were obtained from the cAb-Rep database (https://cab-rep.c2b2.columbia.edu/). The 19-77 and its mutant complexes were first superimposed by using the ‘align’ function in PyMOL 2.5.4. The distance between the Cα from identical residues within the 19-77 heavy chains were then determined using the rms_cur function in PyMOL by in house python script.

### REFERENCES

1. Wang, P. et al. Antibody resistance of SARS-CoV-2 variants B.1.351 and B.1.1.7. Nature 593, 130-135 (2021).
2. Cerutti, G. et al. Potent SARS-CoV-2 neutralizing antibodies directed against spike N-terminal domain target a single supersite. Cell Host Microbe 29, 819-833 e817 (2021).
3. McCallum, M. et al. N-terminal domain antigenic mapping reveals a site of vulnerability for SARS-CoV-2. Cell 184, 2332-2347 e2316 (2021).
4. Wang, P. et al. Increased resistance of SARS-CoV-2 variant P.1 to antibody neutralization. Cell Host Microbe 29, 747-751 e744 (2021).
5. Barnes, C. O. et al. SARS-CoV-2 neutralizing antibody structures inform therapeutic strategies. Nature 588, 682-687 (2020).
6. Planas, D. et al. Reduced sensitivity of SARS-CoV-2 variant Delta to antibody neutralization. Nature 596, 276-280 (2021).
7. Shi, R. et al. A human neutralizing antibody targets the receptor-binding site of SARS-CoV-2. Nature 584, 120-124 (2020).
8. Jones, B. E. et al. The neutralizing antibody, LY-CoV555, protects against SARS-CoV-2 infection in nonhuman primates. Sci Transl Med 13 (2021).
9. Hansen, J. et al. Studies in humanized mice and convalescent humans yield a SARS-CoV-2 antibody cocktail. Science 369, 1010-1014 (2020).
10. Park, Y. J. et al. Antibody-mediated broad sarbecovirus neutralization through ACE2 molecular mimicry. Science 375, 449-454 (2022).
11. Shu, Y. & McCauley, J. GISAID: Global initiative on sharing all influenza data—from vision to reality. Euro Surveill 22 (2017).
12. Scott, L. et al. Track Omicron's spread with molecular data. Science 374, 1454-1455 (2021).
13. Liu, L. et al. Striking antibody evasion manifested by the Omicron variant of SARS-CoV-2. Nature 602, 676-681 (2022).
14. Carreno, J. M. et al. Activity of convalescent and vaccine serum against SARS-CoV-2 Omicron. Nature 602, 682-688 (2022).
15. Cameroni, E. et al. Broadly neutralizing antibodies overcome SARS-CoV-2 Omicron antigenic shift. Nature 602, 664-670 (2022).
16. Planas, D. et al. Considerable escape of SARS-CoV-2 Omicron to antibody neutralization. Nature 602, 671-675 (2022).
17. Cao, Y. et al. Omicron escapes the majority of existing SARS-CoV-2 neutralizing antibodies. Nature 602, 657-663 (2022).
18. Cele, S. et al. Omicron extensively but incompletely escapes Pfizer BNT162b2 neutralization. Nature 602, 654-656 (2022).
19. Zost, S. J. et al. Potently neutralizing and protective human antibodies against SARS-CoV-2. Nature 584, 443-449 (2020).
20. Wang, Q. et al. Antibody evasion by SARS-CoV-2 Omicron subvariants BA.2.12.1, BA.4 and BA. 5. Nature 608, 603-608 (2022).
21. Wang, Q. et al. Alarming antibody evasion properties of rising SARS-CoV-2 BQ and XBB subvariants. Cell 186, 279-286 e278 (2023).
22. Pinto, D. et al. Cross-neutralization of SARS-CoV-2 by a human monoclonal SARS-CoV antibody. Nature 583, 290-295 (2020).
23. Westendorf, K. et al. LY-CoV1404 (bebtelovimab) potently neutralizes SARS-CoV-2 variants. Cell Rep 39, 110812 (2022).
24. Wang, Q. et al. Antibody neutralisation of emerging SARS-CoV-2 subvariants: EG.5.1 and XBC.1.6. Lancet Infect Dis 23, e397-e398 (2023).
25. Yue, C. et al. ACE2 binding and antibody evasion in enhanced transmissibility of XBB.1.5. Lancet Infect Dis 23, 278-280 (2023).
26. Jian, F. et al. Convergent evolution of SARS-CoV-2 XBB lineages on receptor-binding domain 455-456 synergistically enhances antibody evasion and ACE2 binding. PLoS Pathog 19, e1011868 (2023).
27. Wang, Q. et al. Antigenicity and receptor affinity of SARS-CoV-2 BA.2.86 spike. Nature (2023).
28. Hong, Q. et al. Molecular basis of receptor binding and antibody neutralization of Omicron. Nature 604, 546-552 (2022).
29. Guenthoer, J. et al. Identification of broad, potent antibodies to functionally constrained regions of SARS-CoV-2 spike following a breakthrough infection. Proc Natl Acad Sci USA 120, e2220948120 (2023).
30. Liu, L. et al. Antibodies targeting a quaternary site on SARS-CoV-2 spike glycoprotein prevent viral receptor engagement by conformational locking. Immunity 56, 2442-2455 e2448 (2023).
31. Looi, M. K. Covid-19: WHO adds JN.1 as new variant of interest. BMJ 383, 2975 (2023).
32. Wang, Q. et al. XBB.1.5 monovalent mRNA vaccine booster elicits robust neutralizing antibodies against XBB subvariants and JN.1. Cell Host Microbe 32, 315-321 e313 (2024).
33. FDA (2024).
34. Wang, Q. et al. Antibody evasiveness of SARS-CoV-2 subvariants KP.3.1.1 and XEC. bioRxiv, 2024.2011.2017.624037 (2024).
35. Wang, Q., Guo, Y., Ho, J. & Ho, D. D. Activity of Research-Grade Pemivibart against Recent SARS-CoV-2 JN.1 Sublineages. N Engl J Med 391, 1863-1864 (2024).
36. Liu, L. et al. Potent neutralizing antibodies against multiple epitopes on SARS-CoV-2 spike. Nature 584, 450-456 (2020).
37. Yuan, M. et al. Structural basis of a shared antibody response to SARS-CoV-2. Science 369, 1119-1123 (2020).
38. Schymkowitz, J. et al. The FoldX web server: an online force field. Nucleic Acids Res 33, W382-388 (2005).
39. Fenwick, C. et al. Broadly potent anti-SARS-CoV-2 antibody shares 93% of epitope with ACE2 and provides full protection in monkeys. J Infect 87, 524-537 (2023).
40. Cao, Y. et al. Imprinted SARS-CoV-2 humoral immunity induces convergent Omicron RBD evolution. Nature (2022).
41. Nutalai, R. et al. Potent cross-reactive antibodies following Omicron breakthrough in vaccinees. Cell 185, 2116-2131 e2118 (2022).
42. Cao, Y. et al. Potent Neutralizing Antibodies against SARS-CoV-2 Identified by High-Throughput Single-Cell Sequencing of Convalescent Patients' B Cells. Cell 182, 73-84 e16 (2020).
43. Cao, Y. et al. Rational identification of potent and broad sarbecovirus-neutralizing antibody cocktails from SARS convalescents. Cell Rep 41, 111845 (2022).
44. Jian, F. et al. A generalized framework to identify SARS-CoV-2 broadly neutralizing antibodies. bioRxiv, 2024.2004.2016.589454 (2024).
45. Rosen, L. E. et al. A potent pan-sarbecovirus neutralizing antibody resilient to epitope diversification. Cell (2024).
46. Yu, L. et al. Potent and broadly neutralizing antibodies against sarbecoviruses elicited by single ancestral SARS-CoV-2 infection. bioRxiv, 2024.2006.2006.597720 (2024).
47. Yao, T., Ma, Z., Lan, K., Guo, Y. & Liu, L. Neutralizing Activity and Viral Escape of Pemivibart by SARS-CoV-2 JN.1 sublineages. bioRxiv, 2024.2011.2008.622746 (2024).
48. Planas, D. et al. Escape of SARS-CoV-2 Variants KP.1.1, LB.1, and KP3.3 From Approved Monoclonal Antibodies. Pathog Immun 10, 1-11 (2024).
49. Tixagevimab and Cilgavimab (Evusheld) for Pre-Exposure Prophylaxis of COVID-19. JAMA 327, 384-385 (2022).
50. Marks, J. D. et al. By-passing immunization: building high affinity human antibodies by chain shuffling. Biotechnology (N Y) 10, 779-783 (1992).
51. Yoshinaga, K. et al. Ig L-chain shuffling for affinity maturation of phage library-derived human anti-human MCP-1 antibody blocking its chemotactic activity. J Biochem 143, 593-601 (2008).
52. Kim, H. Y., Stojadinovic, A. & Izadjoo, M. J. Affinity maturation of monoclonal antibodies by multi-site-directed mutagenesis. Methods Mol Biol 1131, 407-420 (2014).
53. Banach, B. B. et al. Antibody-directed evolution reveals a mechanism for enhanced neutralization at the HIV-1 fusion peptide site. Nat Commun 14, 7593 (2023).
54. Pan, Y. et al. Screening of potent neutralizing antibodies against SARS-CoV-2 using convalescent patients-derived phage-display libraries. Cell Discov 7, 57 (2021).
55. Hess, G. T. et al. Directed evolution using dCas9-targeted somatic hypermutation in mammalian cells. Nat Methods 13, 1036-1042 (2016).
56. Mason, D. M. et al. High-throughput antibody engineering in mammalian cells by CRISPR/Cas9-mediated homology-directed mutagenesis. Nucleic Acids Res 46, 7436-7449 (2018).
57. Wellner, A. et al. Rapid generation of potent antibodies by autonomous hypermutation in yeast. Nat Chem Biol 17, 1057-1064 (2021).
58. Desautels, T. A. et al. Computationally restoring the potency of a clinical antibody against Omicron. Nature 629, 878-885 (2024).
59. Shan, S. et al. Deep learning guided optimization of human antibody against SARS-CoV-2 variants with broad neutralization. Proc Natl Acad Sci USA 119, e2122954119 (2022).
60. Li, L. et al. Machine learning optimization of candidate antibody yields highly diverse sub-nanomolar affinity antibody libraries. Nat Commun 14, 3454 (2023).
61. Burn Aschner, C. et al. A multi-specific, multi-affinity antibody platform neutralizes sarbecoviruses and confers protection against SARS-CoV-2 in vivo. Sci Transl Med 15, eadf4549 (2023).
62. Song, R., Oren, D. A., Franco, D., Seaman, M. S. & Ho, D. D. Strategic addition of an N-linked glycan to a monoclonal antibody improves its HIV-1-neutralizing activity. Nat Biotechnol 31, 1047-1052 (2013).
63. Ovchinnikov, V., Louveau, J. E., Barton, J. P., Karplus, M. & Chakraborty, A. K. Role of framework mutations and antibody flexibility in the evolution of broadly neutralizing antibodies. Elife 7 (2018).
64. Tramontano, A., Chothia, C. & Lesk, A. M. Framework residue 71 is a major determinant of the position and conformation of the second hypervariable region in the VH domains of immunoglobulins. J Mol Biol 215, 175-182 (1990).
65. Yanaka, S., Moriwaki, Y., Tsumoto, K. & Sugase, K. Elucidation of potential sites for antibody engineering by fluctuation editing. Sci Rep 7, 9597 (2017).
66. Liu, L. et al. An antibody class with a common CDRH3 motif broadly neutralizes sarbecoviruses. Sci Transl Med 14, eabn6859 (2022).
67. Hsieh, C. L. et al. Structure-based design of prefusion-stabilized SARS-CoV-2 spikes. Science 369, 1501-1505 (2020).
68. Chan, K. K. et al. Engineering human ACE2 to optimize binding to the spike protein of SARS coronavirus 2. Science 369, 1261-1265 (2020).
69. Kabsch, W. Integration, scaling, space-group assignment and post-refinement. Acta Crystallogr D Biol Crystallogr 66, 133-144 (2010).
70. Evans, P. R. & Murshudov, G. N. How good are my data and what is the resolution? Acta Crystallogr D Biol Crystallogr 69, 1204-1214 (2013).
71. Winn, M. D. et al. Overview of the CCP4 suite and current developments. Acta Crystallogr D Biol Crystallogr 67, 235-242 (2011).
72. McCoy, A. J. Solving structures of protein complexes by molecular replacement with Phaser. Acta Crystallogr D Biol Crystallogr 63, 32-41 (2007).
73. Emsley, P. & Cowtan, K. Coot: model-building tools for molecular graphics. Acta Crystallogr D Biol Crystallogr 60, 2126-2132 (2004).
74. Adams, P. D. et al. PHENIX: a comprehensive Python-based system for macromolecular structure solution. Acta Crystallogr D Biol Crystallogr 66, 213-221 (2010).
75. Davis, I. W., Murray, L. W., Richardson, J. S. & Richardson, D. C. MOLPROBITY: structure validation and all-atom contact analysis for nucleic acids and their complexes. Nucleic Acids Res 32, W615-619 (2004).
76. Jumper, J. et al. Highly accurate protein structure prediction with AlphaFold. Nature 596, 583-589 (2021).
77. Meng, E. C. et al. UCSF ChimeraX: Tools for structure building and analysis. Protein Sci 32, e4792 (2023).
78. Liebschner, D. et al. Macromolecular structure determination using X-rays, neutrons and electrons: recent developments in Phenix. Acta Crystallogr D Struct Biol 75, 861-877 (2019).

What is claimed is:

1. A monoclonal antibody or an antigen-binding fragment thereof comprising (i) a heavy chain variable domain (VH) and (ii) a light chain variable domain (VL); wherein the monoclonal antibody or antigen binding fragment thereof binds a portion of a receptor-binding domain (RBD) on a spike protein of a sarbecovirus.

2. The monoclonal antibody or antigen-binding fragment thereof of claim 1, wherein the VH domain comprises a heavy chain CDR1 with at least 85%, 90%, 95%, or 99% identity to SEQ ID NO: 3, a heavy chain CDR2 with at least 85%, 90%, 95%, or 99% identity to SEQ ID NO: 4, and a heavy chain CDR3 with at least 85%, 90%, 95%, or 99% identity to SEQ ID NO: 5.

3. The monoclonal antibody or an antigen-binding fragment thereof of claim 1, wherein the VH domain comprises a heavy chain CDR1 identical to SEQ ID NO: 3, a heavy chain CDR2 identical to SEQ ID NO: 4, and a heavy chain CDR3 identical to SEQ ID NO: 5.

4. The monoclonal antibody or antigen-binding fragment thereof of claims 1-3, wherein the VL domain comprises a light chain CDR1 with at least 85%, 90%, 95%, or 99% identity to SEQ ID NO: 6, a light chain CDR2 with at least 85%, 90%, 95%, or 99% identity to SEQ ID NO: 7, and a light chain CDR3 with least 85%, 90%, 95%, or 99% identity to SEQ ID NO: 8.

5. The monoclonal antibody or antigen-binding fragment thereof of claims 1-3, wherein the VL domain comprises a light chain CDR1 identical to SEQ ID NO: 6, a light chain CDR2 identical to SEQ ID NO: 7, and a light chain CDR3 identical to SEQ ID NO: 8.

6. The monoclonal antibody or antigen-binding fragment thereof of claims 1-5, wherein the framework regions of the VH domain comprise at least 85%, 90%, 95%, or 99% identity to the framework regions of SEQ ID NO: 1.

7. The monoclonal antibody or antigen-binding fragment thereof of claims 1-6, wherein the framework regions of the VL domain comprise at least 85%, 90%, 95%, or 99% identity to the framework regions of SEQ ID NO: 2.

8. The monoclonal antibody or antigen-binding fragment thereof of claims 1-5, wherein the framework regions of the VH domain comprise framework regions that are derived from IGHV3-53 of FIG. 3A.

9. The monoclonal antibody or antigen-binding fragment thereof of claims 1-5, or 8, wherein the framework regions of the VL domain comprise framework regions that are derived from IGKV3-11 of FIG. 3B.

10. The monoclonal antibody or antigen-binding fragment thereof of claims 1-5, wherein the framework regions of the VH domain comprise framework regions that are at least 85%, 90%, 95%, or 99% identical to the framework regions of IGHV3-53 of FIG. 3A.

11. The monoclonal antibody or antigen-binding fragment thereof of claims 1-5, or 10, wherein the framework regions of the VL domain comprise framework regions that are at least 85%, 90%, 95%, or 99% identical to the framework regions of FIG. 3B.

12. The monoclonal antibody or antigen-binding fragment thereof of claim 1, wherein the VH domain comprises at least 85%, 90%, 95%, or 99% identity to SEQ ID NO: 1.

13. The monoclonal antibody or antigen-binding fragment thereof of claim 12, wherein the VH domain comprises SEQ ID NO: 1.

14. The monoclonal antibody or antigen-binding fragment thereof of claim 12, wherein the VH domain consists of SEQ ID NO: 1.

15. The monoclonal antibody or antigen-binding fragment thereof of claims 1, 12, 13, or 14, wherein the VL domain comprises at least 85%, 90%, 95%, or 99% identity to SEQ ID NO: 2.

16. The monoclonal antibody or antigen-binding fragment thereof of claim 15, wherein the VL domain comprises SEQ ID NO: 2.

17. The monoclonal antibody or antigen-binding fragment thereof of claim 15, wherein the VL domain consists of SEQ ID NO: 2.

18. The monoclonal antibody or antigen-binding fragment thereof of claims 1-17, wherein the Sarbecovirus is a SARS-CoV or SARS-CoV-2.

19. The monoclonal antibody or antigen-binding fragment thereof of claims 1-17, wherein the Sarbecovirus is a SARS-CoV-2 variant.

20. The monoclonal antibody or antigen-binding fragment thereof of claim 19, wherein the SARS-CoV-2 variant is D614G, B.1.1.7, B.1.351, P.1, B.1.617.2, BA.1, BA.2, BA.5, BA.2.75, CH.1.1, DV.7.1, XBC.1.6, BQ.1.1, XBB.1.5, XBB.1.16.6, XBB.2.3, EG.5.1, FL.1.5.1, JF.1, HV.1, HK.3, BA.2.87.1, BA.2.86, JN.1, JN.4, LB.1, KP.2, KP.3, KP.2.3, KP.3.1.1, XEC, GX-pangolin, RaTG13, BANAL52, BANAL236, or GD-pangolin.

21. The monoclonal antibody or antigen-binding fragment thereof of claim 1, wherein the VH domain comprises SEQ ID NO: 1 and the VL domain comprises SEQ ID NO:2.

22. The monoclonal antibody or antigen-binding fragment thereof of claim 1, wherein the VH domain comprises at least 85%, 90%, 95%, or 99% identity to SEQ ID NO: 1 and the VL domain comprises at least 85%, 90%, 95%, or 99% identity to SEQ ID NO:2.

23. The monoclonal antibody or antigen-binding fragment thereof of claim 22, wherein the heavy chain CDR1, CDR2, and CDR3 of the monoclonal antibody of antigen-binding fragment thereof has an increased RMSF score as compared to heavy chain CDR1, CDR2, and CDR3 of a monoclonal antibody or fragment thereof comprising SEQ ID NO:1 and SEQ ID NO: 2.

24. The monoclonal antibody or antigen-binding fragment thereof of claim 23, wherein the average RMSF score is above 1.0.

25. The monoclonal antibody or antigen-binding fragment thereof of claim 23, wherein the average RMSF score is above 1.2.

26. The monoclonal antibody or antigen-binding fragment thereof of claim 1, wherein the VH domain comprises at least 85%, 90%, 95%, or 99% identity to SEQ ID NO: 1 and comprises an amino acid mutation at position 71.

27. The monoclonal antibody or antigen-binding fragment thereof of claim 1, wherein the VH domain comprises SEQ ID NO:1 comprising an amino acid mutation at position 71.

28. The monoclonal antibody or antigen-binding fragment thereof of claim 1, wherein the VH domain consists of SEQ ID NO: 1 with an amino acid mutation at position 71.

29. The monoclonal antibody or antigen-binding fragment thereof of claims 26-28, wherein the VL domain comprises at least 85%, 90%, 95%, or 99% identity to SEQ ID NO: 2.

30. The monoclonal antibody or antigen-binding fragment thereof of claim 29, wherein the VL domain comprises SEQ ID NO: 2.

31. The monoclonal antibody or antigen-binding fragment thereof of claim 29, wherein the VL domain consists of SEQ ID NO: 2.

32. The monoclonal antibody or antigen-binding fragment thereof of claims 26-31, wherein the amino acid mutation is R71A, R71C, R71D, R71E, R71F, R71G, R71H, R71I, R71K, R71L, R71M, R71N, R71P, R71Q, R71S, R71T, R71V, R71W, or R71Y.

33. The monoclonal antibody or antigen-binding fragment thereof of claim 32, wherein the amino acid mutation is R71A.

34. The monoclonal antibody or antigen-binding fragment thereof of claim 32, wherein the amino acid mutation is R71L.

35. The monoclonal antibody or antigen-binding fragment thereof of claim 32, wherein the amino acid mutation is R71V.

36. The monoclonal antibody or antigen-binding fragment thereof of claims 1-35, wherein the monoclonal antibody or antigen-binding fragment thereof is a single chain antibody, Fab, Fab′, F(ab′)2, Fv or scFv.

37. The monoclonal antibody or antigen-binding fragment thereof of claims 1-35, wherein the monoclonal antibody is an IgG type.

38. The monoclonal antibody or antigen-binding fragment thereof of claims 1-35, wherein the monoclonal antibody is an IgA type.

39. The monoclonal antibody or antigen-binding fragment thereof of claims 1-35, comprising a monoclonal antibody comprising:
a first arm comprising the VH domain and VL domain, wherein the VH and VL domain form a binding site that binds a portion of the RBD on the spike protein of a sarbecovirus; and
a second arm comprising a second VH domain, identical to the VH domain of the first arm, and a second VL domain, identical to the VL domain of the first arm, wherein the second VH and second VL form a binding site that binds a portion of the RBD on the spike protein of a sarbecovirus.

40. The monoclonal antibody or antigen-binding fragment thereof of claim 39, wherein the first arm and the second arm each further comprise a CH1 domain, a hinge domain, and a CL domain.

41. The monoclonal antibody or antigen-binding fragment thereof of claim 39, wherein:
the VH domain of the first arm is encoded by a first polypeptide chain,
the VL domain of the first arm is encoded by a second polypeptide chain,
the second VH domain of the second arm is encoded by a third polypeptide chain,
the second VL domain of the second arm is encoded by a fourth polypeptide chain.

42. The monoclonal antibody or antigen-binding fragment thereof of claim 41, wherein the first polypeptide chain and the third polypeptide chain each further encode a hinge domain, a CH1 domain, the Fc domain, and wherein the second polypeptide chain and the fourth polypeptide chain each further encode a CL domain.

43. The monoclonal antibody or antigen-binding fragment thereof of claim 42, wherein the first polypeptide chain and the third polypeptide chain comprise the same sequence, and wherein the second polypeptide chain and the fourth polypeptide chain comprise the same sequence.

44. The monoclonal antibody or antigen-binding fragment thereof of claims 41-43, wherein the first polypeptide chain and the second polypeptide chain are linked by one or more covalent disulfide bonds and the third polypeptide chain and the fourth polypeptide chain are linked by one or more covalent disulfide bonds, and the first polypeptide chain and the third polypeptide chain are linked by one or more covalent disulfide bonds.

45. The monoclonal antibody or antigen-binding fragment thereof of claims 37-44, comprising an engineered constant domain.

46. A pharmaceutical composition comprising: monoclonal antibody or antigen-binding fragment thereof of any of claims 1-45; and a pharmaceutically acceptable carrier.

47. A method of treating or preventing a Sarbecovirus infection in a subject in need thereof, the method comprising administering to the subject a therapeutically effective amount of the pharmaceutical composition of claim 46.

48. The method of claim 47, wherein the Sarbecovirus is a SARS-CoV or SARS-CoV-2.

49. The method of claim 47, wherein the Sarbecovirus is a SARS-CoV-2 variant.

50. The method of claim 49, wherein the SARS-CoV-2 variant is D614G, B.1.1.7, B.1.351, P.1, B.1.617.2, BA.1, BA.2, BA.5, BA.2.75, CH.1.1, DV.7.1, XBC.1.6 variant, BQ.1.1, XBB.1.5, XBB.1.16.6, XBB.2.3, EG.5.1, FL.1.5.1, JF.1, HV.1, HK.3, BA.2.87.1, BA.2.86, JN.1, JN.4, LB.1, KP.2, KP.3, KP.2.3, KP.3.1.1, XEC, GX-pangolin, RaTG13, BANAL52, BANAL236, or GD-pangolin.

51. The method of claim 47, wherein the pharmaceutical composition is administered in combination with an anti-viral medicament.

52. The method of claim 48, wherein the anti-viral medicament comprises a second anti-sarbecovirus antibody.

53. The method of claim 49, wherein the second anti-sarbecovirus antibody comprises CYFN1006-146.

54. A nucleic acid comprising a nucleotide sequence encoding the monoclonal antibody or antigen binding fragment thereof of claims 1-45.

55. A vector comprising the nucleic acid molecule of claim 54.

56. A cell comprising the vector of claim 55.

57. A cell expressing the monoclonal antibody or antigen-binding fragment thereof of claims 1-45.

58. A pharmaceutical composition comprising the nucleic acid of claim 54 or vector of claim 55.

59. A composition comprising: one or more vectors comprising a nucleotide sequence encoding the monoclonal antibody or antigen binding fragment thereof of any of claims 1-45.

60. A means for binding a portion of the RBD on a spike protein of a sarbecovirus.

61. The means of claim 60, wherein the means comprises the monoclonal antibody or antigen binding fragment thereof of any of claims 1-45.

62. One or more vectors comprising:
a first vector comprising a nucleotide sequence encoding the VH chain of the monoclonal antibody of any of claims 10-14 and
a second vector comprising a nucleotide sequence encoding the VL chain of the monoclonal antibody of any of claims 15-19.

63. The one or more vectors of claim 62, wherein the first and second vectors are the same vector.

64. The one or more vectors of claim 62, wherein the first and second vectors are two different vectors.

65. One or more host cells comprising:
a first vector comprising a polynucleotide sequence encoding the VH chain of the monoclonal antibody or antigen binding fragment thereof of any of claims 10-14; and
a second vector comprising a polynucleotide sequence encoding the VH chain of the monoclonal antibody or antigen binding fragment thereof of any of claims 15-19.

66. The one or more host cells of claim 65, wherein the first vector and the second vector are the same vector.

67. The one or more host cells of claim 65, wherein the first vector and the second vector are two different vectors.

68. The method of claims 47-53, wherein the subject is a human.


SEQUENCE LISTING (8 sequences)

SEQ ID NO: 1 - protein, 115 aa, synthetic construct
EVQVVESGGGLIQPGGSLRLSCTASELIISRNYMSWVRQAPGKGLEWLSVIYPGGSSFYTDSLKGRFTISRDNSKNTLYLQMNRLGVEDTAIYYCVRDAPSESDWGQGTLVTVSS

SEQ ID NO: 2 - protein, 108 aa, synthetic construct
EIVLTQSPATLSLFPGERATLSCRASQNIGHFLTWYQQKPGQAPRLLIYDASNRATGVPARFSGSGSETEFTLTISSLGPEDFAVYYCQERSDWPRGTFGQGTKVEIK

SEQ ID NO: 3 - protein, 8 aa, synthetic construct
ELIISRNY

SEQ ID NO: 4 - protein, 7 aa, synthetic construct
IYPGGSS

SEQ ID NO: 5 - protein, 9 aa, synthetic construct
VRDAPSESD

SEQ ID NO: 6 - protein, 6 aa, synthetic construct
QNIGHF

SEQ ID NO: 7 - skipped

SEQ ID NO: 8 - protein, 10 aa, synthetic construct
QERSDWPRGT